SEQUENCE LISTING (85 sequences)

SEQ ID NO: 1 - DNA, 474 bp, Escherichia coli
atgaaaggtgatactaaagttataaattatctcaacaaactgttgggaaatgagcttgtcgcaatcaatcagtactttctccatgcccgaatgtttaaaaactggggtctcaaacgtctcaatgatgtggagtatcatgaatccattgatgagatgaaacacgccgatcgttatattgagcgcattctttttctggaaggtcttccaaacttacaggacctgggcaaactgaacattggtgaagatgttgaggaaatgctgcgttctgatctggcacttgagctggatggcgcgaagaatttgcgtgaggcaattggttatgccgatagcgttcatgattacgtcagccgcgatatgatgatagaaattttgcgtgatgaagaaggccatatcgactggctggaaacggaacttgatctgattcagaagatgggcctgcaaaattatctgcaagcacagatccgcgaagaaggt

SEQ ID NO: 2 - protein, 158 aa, Escherichia coli
MKGDTKVINYLNKLLGNELVAINQYFLHARMFKNWGLKRLNDVEYHESIDEMKHADRYIERILFLEGLPNLQDLGKLNIGEDVEEMLRSDLALELDGAKNLREAIGYADSVHDYVSRDMMIEILRDEEGHIDWLETELDLIQKMGLQNYLQAQIREEG

SEQ ID NO: 3 - DNA, 33 bp, Artificial Sequence
  The nucleotide sequence for a  “His-tag”
atgggcagccatcaccatcaccaccatagcggc

SEQ ID NO: 4 - protein, 11 aa, Artificial Sequence
  The amino acid sequence for a “His-tag”
MGSHHHHHHSG

SEQ ID NO: 5 - DNA, 525 bp, Artificial Sequence
  The nucleoitde sequence of a bacterioferritin peptide comprising an N-terminal “His-tag”
atgggcagccatcaccatcaccaccatagcggcgaaaacctgtactttcagatgaaaggtgatactaaagttataaattatctcaacaaactgttgggaaatgagcttgtcgcaatcaatcagtactttctccatgcccgaatgtttaaaaactggggtctcaaacgtctcaatgatgtggagtatcatgaatccattgatgagatgaaacacgccgatcgttatattgagcgcattctttttctggaaggtcttccaaacttacaggacctgggcaaactgaacattggtgaagatgttgaggaaatgctgcgttctgatctggcacttgagctggatggcgcgaagaatttgcgtgaggcaattggttatgccgatagcgttcatgattacgtcagccgcgatatgatgatagaaattttgcgtgatgaagaaggccatatcgactggctggaaacggaacttgatctgattcagaagatgggcctgcaaaattatctgcaagcacagatccgcgaagaaggt

SEQ ID NO: 6 - protein, 175 aa, Artificial Sequence
  An amino acid sequence of a bacterioferritin peptide comprising an N-terminal “His-tag”
MGSHHHHHHSGENLYFQMKGDTKVINYLNKLLGNELVAINQYFLHARMFKNWGLKRLNDVEYHESIDEMKHADRYIERILFLEGLPNLQDLGKLNIGEDVEEMLRSDLALELDGAKNLREAIGYADSVHDYVSRDMMIEILRDEEGHIDWLETELDLIQKMGLQNYLQAQIREEG

SEQ ID NO: 7 - DNA, 42 bp, Unknown
  A nucelotide sequence of a gold-binding peptide
atgcacggtaaaacccaggcgacctctggtaccatccagtct

SEQ ID NO: 8 - protein, 14 aa, Unknown
  An amino acid sequence of a gold-binding peptide
MHGKTQATSGTIQS

SEQ ID NO: 9 - DNA, 522 bp, Artificial Sequence
  A nucleotide sequence of a variant bacterioferritin comprising a C-terminal nucleating agent binding peptide
atgaaaggtgatactaaagttataaattatctcaacaaactgttgggaaatgagcttgtcgcaatcaatcagtactttctccatgcccgaatgtttaaaaactggggtctcaaacgtctcaatgatgtggagtatcatgaatccattgatgagatgaaacacgccgatcgttatattgagcgcattctttttctggaaggtcttccaaacttacaggacctgggcaaactgaacattggtgaagatgttgaggaaatgctgcgttctgatctggcacttgagctggatggcgcgaagaatttgcgtgaggcaattggttatgccgatagcgttcatgattacgtcagccgcgatatgatgatagaaattttgcgtgatgaagaaggccatatcgactggctggaaacggaacttgatctgattcagaagatgggcctgcaaaattatctgcaagcacagatccgcgaagaaggtaccggaatgcacggtaaaacccaggcgacctctggtaccatccagtct

SEQ ID NO: 10 - protein, 174 aa, Artificial Sequence
  An amino acid sequence of a variant bacterioferritin comprising a C-terminal nucleating agent binding peptide
MKGDTKVINYLNKLLGNELVAINQYFLHARMFKNWGLKRLNDVEYHESIDEMKHADRYIERILFLEGLPNLQDLGKLNIGEDVEEMLRSDLALELDGAKNLREAIGYADSVHDYVSRDMMIEILRDEEGHIDWLETELDLIQKMGLQNYLQAQIREEGTGMHGKTQATSGTIQS

SEQ ID NO: 11 - DNA, 573 bp, Artificial Sequence
  A nucleotide sequnece of a variant bacterioferritin comprising an N-terminal “His-tag” and a C-terminal nucleating agent binding peptide
atgggcagccatcaccatcaccaccatagcggcgaaaacctgtactttcagatgaaaggtgatactaaagttataaattatctcaacaaactgttgggaaatgagcttgtcgcaatcaatcagtactttctccatgcccgaatgtttaaaaactggggtctcaaacgtctcaatgatgtggagtatcatgaatccattgatgagatgaaacacgccgatcgttatattgagcgcattctttttctggaaggtcttccaaacttacaggacctgggcaaactgaacattggtgaagatgttgaggaaatgctgcgttctgatctggcacttgagctggatggcgcgaagaatttgcgtgaggcaattggttatgccgatagcgttcatgattacgtcagccgcgatatgatgatagaaattttgcgtgatgaagaaggccatatcgactggctggaaacggaacttgatctgattcagaagatgggcctgcaaaattatctgcaagcacagatccgcgaagaaggtaccggaatgcacggtaaaacccaggcgacctctggtaccatccagtct

SEQ ID NO: 12 - protein, 191 aa, Artificial Sequence
  An amino acid sequence of a variant bacterioferritin comprising an N-terminal “His-tag” and a C-terminal nucleating agent binding peptide.
MGSHHHHHHSGENLYFQMKGDTKVINYLNKLLGNELVAINQYFLHARMFKNWGLKRLNDVEYHESIDEMKHADRYIERILFLEGLPNLQDLGKLNIGEDVEEMLRSDLALELDGAKNLREAIGYADSVHDYVSRDMMIEILRDEEGHIDWLETELDLIQKMGLQNYLQAQIREEGTGMHGKTQATSGTIQS

SEQ ID NO: 13 - DNA, 123 bp, Artificial Sequence
  A nucleotide sequence encoding a construct comprising a promoter, a ribosomal binding site (RBS) and nucleic acid encoding a His tag, wherein the promoter is a combination of a constitutive J23100 promoter and an inducible T7 promoter.
ttgacggctagctcagtcctaggtacagtgctagctaatacgactcactatagggagatactagagaaatcaaattaaggaggtaagataatgggcagccatcaccatcaccaccatagcggc

SEQ ID NO: 14 - protein, 11 aa, Artificial Sequence
  A amino acid sequence of the “His-tag” of a construct comprising a promoter, a ribosomal binding site (RBS) and nucleic acid encoding a His tag.
MGSHHHHHHSG

SEQ ID NO: 15 - DNA, 555 bp, Escherichia coli
atgaccacggcgtctactagccaggtccgccaaaactatcatcaggacagcgaggcggcgatcaatcgccagattaacctggagttgtacgcaagctacgtttacctgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtatctttctgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccggg

SEQ ID NO: 16 - protein, 185 aa, Escherichia coli
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPG

SEQ ID NO: 17 - DNA, 531 bp, Escherichia coli
atgtctagccaaattcgccagaattacagcaccgacgttgaagcggcagtcaacagcctggttaatctgtacttgcaggccagctatacgtatctgagcctgggcttttactttgaccgcgacgatgtggccttggaaggcgtgagccactttttccgtgagctggcggaagagaaacgcgaaggctatgagcgcctgctgaaaatgcagaaccaacgtggcggtcgtgctctgttccaagacatcaagaaaccggcggaagatgagtggggtaaaaccccggatgcgatgaaggccgcaatggctttggagaagaaactgaatcaggcactgctggatctgcacgcgctgggttccgcacgtaccgacccgcacctgtgcgatttcttggaaacgcattttctggacgaagaggtcaagctgatcaagaaaatgggcgaccacctgacgaacttgcatcgtctgggtggtccagaggcgggtctgggtgagtacctgttcgagcgtctgactctgaagcatgatcccggg

SEQ ID NO: 18 - protein, 177 aa, Escherichia coli
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHDPG

SEQ ID NO: 19 - protein, 5 aa, Artificial Sequence
  Modified hydrophopbic residues in dimer interface of ferritin heavy chain
GRIFL

SEQ ID NO: 20 - protein, 5 aa, Artificial Sequence
  Modified hydrophopbic residues in dimer interface of ferritin heavy chain
LELYA

SEQ ID NO: 21 - protein, 5 aa, Artificial Sequence
  Modified hydrophopbic residues in dimer interface of ferritin heavy chain
VYLSM

SEQ ID NO: 22 - protein, 5 aa, Artificial Sequence
  Modified hydrophopbic residues in dimer interface of ferritin heavy chain
IFLQD

SEQ ID NO: 23 - protein, 5 aa, Artificial Sequence
  Modified hydrophopbic residues in dimer interface of ferritin heavy chain
RLLKM

SEQ ID NO: 24 - protein, 5 aa, Artificial Sequence
  Modified hydrophopbic residues in dimer interface of ferritin heavy chain
LYLQA

SEQ ID NO: 25 - protein, 5 aa, Artificial Sequence
  Modified hydrophopbic residues in dimer interface of ferritin heavy chain
TYLSL

SEQ ID NO: 26 - protein, 5 aa, Artificial Sequence
  Modified hydrophopbic residues in dimer interface of ferritin heavy chain
ALFQD

SEQ ID NO: 27 - protein, 5 aa, Artificial Sequence
  Modified hydrophopbic residues in dimer interface of ferritin heavy chain
LGFYF

SEQ ID NO: 28 - protein, 5 aa, Artificial Sequence
  Modified hydrophopbic residues in dimer interface of ferritin heavy chain
DEWGK

SEQ ID NO: 29 - protein, 5 aa, Artificial Sequence
  Modified hydrophopbic residues in dimer interface of ferritin heavy chain
QDIKK

SEQ ID NO: 30 - DNA, 555 bp, Artificial Sequence
  A nucleotide sequence of a human heavy chain ferritin polypeptide
atgaccacggcgtctactagccaggtccgccaaaactatcatcaggacagcgaggcggcgatcaatcgccagattaacctggaggcgtacgcaagctacgtttacgcgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtgcgtttgcgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccggg

SEQ ID NO: 31 - protein, 185 aa, Artificial Sequence
  An amino acid sequence of a human heavy chain ferritin polypeptide
MTTASTSQVRQNYHQDSEAAINRQINLEAYASYVYASMSYYFDRDDVALKNFAKYFLHQSHEEREHAEKLMKLQNQRGGRAFAQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPG

SEQ ID NO: 32 - DNA, 531 bp, Artificial Sequence
  A nucleotide sequence of a human light chain ferritin polypeptide
atgtctagccaaattcgccagaattacagcaccgacgttgaagcggcagtcaacagcctggttaatctgtacttgcaggccagctatacgtatgcgagcctgggcgcgtactttgaccgcgacgatgtggccttggaaggcgtgagccactttttccgtgagctggcggaagagaaacgcgaaggctatgagcgcctggcgaaaatgcagaaccaacgtggcggtcgtgctctggcgcaagacatcaagaaaccggcggaagatgagtggggtaaaaccccggatgcgatgaaggccgcaatggctttggagaagaaactgaatcaggcactgctggatctgcacgcgctgggttccgcacgtaccgacccgcacctgtgcgatttcttggaaacgcattttctggacgaagaggtcaagctgatcaagaaaatgggcgaccacctgacgaacttgcatcgtctgggtggtccagaggcgggtctgggtgagtacctgttcgagcgtctgactctgaagcatgatcccggg

SEQ ID NO: 33 - protein, 177 aa, Artificial Sequence
  An amino acid sequence of a human light chain ferritin polypeptide
MSSQIRQNYSTDVEAAVNSLVNLYLQASYTYASLGAYFDRDDVALEGVSHFFRELAEEKREGYERLAKMQNQRGGRALAQDIKKPAEDEWGKTPDAMKAAMALEKKLNQALLDLHALGSARTDPHLCDFLETHFLDEEVKLIKKMGDHLTNLHRLGGPEAGLGEYLFERLTLKHDPG

SEQ ID NO: 34 - DNA, 714 bp, Unknown
  A nucelotide sequence of GFP
atgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataag

SEQ ID NO: 35 - protein, 238 aa, Unknown
  An amino acid sequence of GFP
MRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 36 - DNA, 1299 bp, Artificial Sequence
  A nucleotide sequence of a heavy chain ferritin peptide comprising an N-terminal fluorophore
atgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgaccacggcgtctactagccaggtccgccaaaactatcatcaggacagcgaggcggcgatcaatcgccagattaacctggaggcgtacgcaagctacgtttacgcgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtgcgtttgcgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccggg

SEQ ID NO: 37 - protein, 433 aa, Artificial Sequence
  An amino acid sequence of a heavy chain ferritin peptide comprising an N-terminal fluorophore
MRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMTTASTSQVRQNYHQDSEAAINRQINLEAYASYVYASMSYYFDRDDVALKNFAKYFLHQSHEEREHAEKLMKLQNQRGGRAFAQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPG

SEQ ID NO: 38 - DNA, 1275 bp, Artificial Sequence
  A nucleotide sequence of a light chain ferritin peptide comprising an N-terminal fluorophore
atgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgtctagccaaattcgccagaattacagcaccgacgttgaagcggcagtcaacagcctggttaatctgtacttgcaggccagctatacgtatgcgagcctgggcgcgtactttgaccgcgacgatgtggccttggaaggcgtgagccactttttccgtgagctggcggaagagaaacgcgaaggctatgagcgcctggcgaaaatgcagaaccaacgtggcggtcgtgctctggcgcaagacatcaagaaaccggcggaagatgagtggggtaaaaccccggatgcgatgaaggccgcaatggctttggagaagaaactgaatcaggcactgctggatctgcacgcgctgggttccgcacgtaccgacccgcacctgtgcgatttcttggaaacgcattttctggacgaagaggtcaagctgatcaagaaaatgggcgaccacctgacgaacttgcatcgtctgggtggtccagaggcgggtctgggtgagtacctgttcgagcgtctgactctgaagcatgatcccggg

SEQ ID NO: 39 - protein, 425 aa, Artificial Sequence
  An amino acid sequence of a heavy chain ferritin peptide comprising an N-terminal fluorophore
MRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMSSQIRQNYSTDVEAAVNSLVNLYLQASYTYASLGAYFDRDDVALEGVSHFFRELAEEKREGYERLAKMQNQRGGRALAQDIKKPAEDEWGKTPDAMKAAMALEKKLNQALLDLHALGSARTDPHLCDFLETHFLDEEVKLIKKMGDHLTNLHRLGGPEAGLGEYLFERLTLKHDPG

SEQ ID NO: 40 - DNA, 1380 bp, Artificial Sequence
  A nucleotide sequence of a heavy chain ferritin peptide comprising an N-terminal fluorophore
atgggcagccatcaccatcaccaccatagcggcgaaaacctgtactttcagggtggaggaggctctggtggaggcgccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgaccacggcgtctactagccaggtccgccaaaactatcatcaggacagcgaggcggcgatcaatcgccagattaacctggaggcgtacgcaagctacgtttacgcgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtgcgtttgcgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccggg

SEQ ID NO: 41 - protein, 460 aa, Artificial Sequence
  An amino acid sequence of a heavy chain ferritin peptide comprising an N-terminal fluorophore
MGSHHHHHHSGENLYFQGGGGSGGGAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMTTASTSQVRQNYHQDSEAAINRQINLEAYASYVYASMSYYFDRDDVALKNFAKYFLHQSHEEREHAEKLMKLQNQRGGRAFAQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPG

SEQ ID NO: 42 - DNA, 1356 bp, Artificial Sequence
  A nucleotide sequence of a light chain ferritin peptide comprising an N-terminal fluorophore
atgggcagccatcaccatcaccaccatagcggcgaaaacctgtactttcagggtggaggaggctctggtggaggcgccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgtctagccaaattcgccagaattacagcaccgacgttgaagcggcagtcaacagcctggttaatctgtacttgcaggccagctatacgtatgcgagcctgggcgcgtactttgaccgcgacgatgtggccttggaaggcgtgagccactttttccgtgagctggcggaagagaaacgcgaaggctatgagcgcctggcgaaaatgcagaaccaacgtggcggtcgtgctctggcgcaagacatcaagaaaccggcggaagatgagtggggtaaaaccccggatgcgatgaaggccgcaatggctttggagaagaaactgaatcaggcactgctggatctgcacgcgctgggttccgcacgtaccgacccgcacctgtgcgatttcttggaaacgcattttctggacgaagaggtcaagctgatcaagaaaatgggcgaccacctgacgaacttgcatcgtctgggtggtccagaggcgggtctgggtgagtacctgttcgagcgtctgactctgaagcatgatcccggg

SEQ ID NO: 43 - protein, 452 aa, Artificial Sequence
  An amino acid sequence of a light chain ferritin peptide comprising an N-terminal fluorophore
MGSHHHHHHSGENLYFQGGGGSGGGAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMSSQIRQNYSTDVEAAVNSLVNLYLQASYTYASLGAYFDRDDVALEGVSHFFRELAEEKREGYERLAKMQNQRGGRALAQDIKKPAEDEWGKTPDAMKAAMALEKKLNQALLDLHALGSARTDPHLCDFLETHFLDEEVKLIKKMGDHLTNLHRLGGPEAGLGEYLFERLTLKHDPG

SEQ ID NO: 44 - DNA, 1422 bp, Artificial Sequence
  A nucleotide sequence of a heavy chain ferritin peptide comprising an N-terminal fluorophore
atgggcagccatcaccatcaccaccatagcggcgaaaacctgtactttcagggtggaggaggctctggtggaggcgccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgaccacggcgtctactagccaggtccgccaaaactatcatcaggacagcgaggcggcgatcaatcgccagattaacctggaggcgtacgcaagctacgtttacgcgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtgcgtttgcgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccgggatgcacggtaaaacccaggcgacctctggtaccatccagtct

SEQ ID NO: 45 - protein, 474 aa, Artificial Sequence
  An amino acid sequence of a heavy chain ferritin peptide comprising an N-terminal fluorophore
MGSHHHHHHSGENLYFQGGGGSGGGAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMTTASTSQVRQNYHQDSEAAINRQINLEAYASYVYASMSYYFDRDDVALKNFAKYFLHQSHEEREHAEKLMKLQNQRGGRAFAQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPGMHGKTQATSGTIQS

SEQ ID NO: 46 - DNA, 1398 bp, Artificial Sequence
  A nucleotide sequence of a light chain ferritin peptide comprising an N-terminal fluorophore
atgggcagccatcaccatcaccaccatagcggcgaaaacctgtactttcagggtggaggaggctctggtggaggcgccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgtctagccaaattcgccagaattacagcaccgacgttgaagcggcagtcaacagcctggttaatctgtacttgcaggccagctatacgtatgcgagcctgggcgcgtactttgaccgcgacgatgtggccttggaaggcgtgagccactttttccgtgagctggcggaagagaaacgcgaaggctatgagcgcctgaaaatgcagaaccaacgtggcggtcgtgctctggcgcaagacatcaagaaaccggcggcggaagatgagtggggtaaaaccccggatgcgatgaaggccgcaatggctttggagaagaaactgaatcaggcactgctggatctgcacgcgctgggttccgcacgtaccgacccgcacctgtgcgatttcttggaaacgcattttctggacgaagaggtcaagctgatcaagaaaatgggcgaccacctgacgaacttgcatcgtctgggtggtccagaggcgggtctgggtgagtacctgttcgagcgtctgactctgaagcatgatcccgggatgcacggtaaaacccaggcgacctctggtaccatccagtct

SEQ ID NO: 47 - protein, 466 aa, Artificial Sequence
  An amino acid sequence of a light chain ferritin peptide comprising an N-terminal fluorophore
MGSHHHHHHSGENLYFQGGGGSGGGAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMSSQIRQNYSTDVEAAVNSLVNLYLQASYTYASLGAYFDRDDVALEGVSHFFRELAEEKREGYERLAKMQNQRGGRALAQDIKKPAEDEWGKTPDAMKAAMALEKKLNQALLDLHALGSARTDPHLCDFLETHFLDEEVKLIKKMGDHLTNLHRLGGPEAGLGEYLFERLTLKHDPGMHGKTQATSGTIQS

SEQ ID NO: 48 - DNA, 348 bp, Unknown
  The nucleotide sequence of a ZZ domain
gataataaatttaacaaagaacagcaaaacgcgttttacgagattctgcacctgccgaatctgaatgaagagcagcgtaatgccttcatccagagcctgaaagatgatccgagccagagcgcgaacctgctggccgaagcgaaaaaactgaatgacgcgcaggccccgaaagtggacaacaaattcaataaagaacaacagaatgccttctacgagatcctgcatctgccgaacctgaatgaagaacagcgcaatgcctttatccagagcctgaaagatgatccgagccagagcgccaatctgctggccgaagccaaaaaactgaacgatgcgcaagcgccgaaagtg

SEQ ID NO: 49 - protein, 116 aa, Unknown
  The amino acid sequence of a ZZ domain
DNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKV

SEQ ID NO: 50 - DNA, 1782 bp, Artificial Sequence
  A nucleotide sequence of a human heavy chain ferritin peptide
atgggcagccatcaccatcaccaccatagcggcggtacgggcagcagcggtgccactgcaggtggtagcgataataaatttaacaaagaacagcaaaacgcgttttacgagattctgcacctgccgaatctgaatgaagagcagcgtaatgccttcatccagagcctgaaagatgatccgagccagagcgcgaacctgctggccgaagcgaaaaaactgaatgacgcgcaggccccgaaagtggacaacaaattcaataaagaacaacagaatgccttctacgagatcctgcatctgccgaacctgaatgaagaacagcgcaatgcctttatccagagcctgaaagatgatccgagccagagcgccaatctgctggccgaagccaaaaaactgaacgatgcgcaagcgccgaaagtgggcagcggcggtggtggaggaggctctggtggaggctggagccacccgcagttcgaaaaagccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttcgtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgaccacggcgtctactagccaggtccgccaaaactatcatcaggacagcgaggcggcgatcaatcgccagattaacctggaggcgtacgcaagctacgtttacgcgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtgcgtttgcgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccggg

SEQ ID NO: 51 - protein, 594 aa, Artificial Sequence
  An amino acid sequence of a human heavy chain ferritin peptide
MGSHHHHHHSGGTGSSGATAGGSDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVGSGGGGGGSGGGWSHPQFEKAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMTTASTSQVRQNYHQDSEAAINRQINLEAYASYVYASMSYYFDRDDVALKNFAKYFLHQSHEEREHAEKLMKLQNQRGGRAFAQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPG

SEQ ID NO: 52 - DNA, 1767 bp, Artificial Sequence
  A nucleotide sequence of a bacterioferritin peptide
atgggcagccatcaccatcaccaccatagcggcggtacgggcagcagcggtgccactgcaggtggtagcgataataaatttaacaaagaacagcaaaacgcgttttacgagattctgcacctgccgaatctgaatgaagagcagcgtaatgccttcatccagagcctgaaagatgatccgagccagagcgcgaacctgctggccgaagcgaaaaaactgaatgacgcgcaggccccgaaagtggacaacaaattcaataaagaacaacagaatgccttctacgagatcctgcatctgccgaacctgaatgaagaacagcgcaatgcctttatccagagcctgaaagatgatccgagccagagcgccaatctgctggccgaagccaaaaaactgaacgatgcgcaagcgccgaaagtgggcagcggcggtggtggaggaggctctggtggaggctggagccacccgcagttcgaaaaagccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtggagggggttgcaccggcatgaaaggtgatactaaagttataaattatctcaacaaactgttgggaaatgagcttgtcgcaatcaatcagtactttctccatgcccgaatgtttaaaaactggggtctcaaacgtctcaatgatgtggagtatcatgaatccattgatgagatgaaacacgccgatcgttatattgagcgcattctttttctggaaggtcttccaaacttacaggacctgggcaaactgaacattggtgaagatgttgaggaaatgctgcgttctgatctggcacttgagctggatggcgcgaagaatttgcgtgaggcaattggttatgccgatagcgttcatgattacgtcagccgcgatatgatgatagaaattttgcgtgatgaagaaggccatatcgactggctggaaacggaacttgatctgattcagaagatgggcctgcaaaattatctgcaagcacagatccgcgaagaaggtaccggaatgcacggtaaaacccaggcgacctctggtaccatccagtct

SEQ ID NO: 53 - protein, 589 aa, Artificial Sequence
  An amino acid sequence of a bacterioferritin peptide
MGSHHHHHHSGGTGSSGATAGGSDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVGSGGGGGGSGGGWSHPQFEKAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGGGGCTGMKGDTKVINYLNKLLGNELVAINQYFLHARMFKNWGLKRLNDVEYHESIDEMKHADRYIERILFLEGLPNLQDLGKLNIGEDVEEMLRSDLALELDGAKNLREAIGYADSVHDYVSRDMMIEILRDEEGHIDWLETELDLIQKMGLQNYLQAQIREEGTGMHGKTQATSGTIQS

SEQ ID NO: 54 - DNA, 1380 bp, Artificial Sequence
  A nucleotide sequence of a fusion protein comprising a wild-type heavy chain human ferritin, GFP and a His tag
atgggcagccatcaccatcaccaccatagcggcgaaaacctgtactttcagggtggaggaggctctggtggaggcgccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgaccacggcgtctactagccaggtccgccaaaactatcatcaggacagcgaggcggcgatcaatcgccagattaacctggagttgtacgcaagctacgtttacctgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtatctttctgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccggg

SEQ ID NO: 55 - protein, 460 aa, Artificial Sequence
  An amino acid sequence of a fusion protein comprising a wild-type heavy chain human ferritin, GFP and a His tag
MGSHHHHHHSGENLYFQGGGGSGGGAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPG

SEQ ID NO: 56 - DNA, 1356 bp, Artificial Sequence
  A nucleotide sequence of a fusion protein comprising a wild-type heavy chain human ferritin, GFP and a His tag
atgggcagccatcaccatcaccaccatagcggcgaaaacctgtactttcagggtggaggaggctctggtggaggcgccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgtctagccaaattcgccagaattacagcaccgacgttgaagcggcagtcaacagcctggttaatctgtacttgcaggccagctatacgtatctgagcctgggcttttactttgaccgcgacgatgtggccttggaaggcgtgagccactttttccgtgagctggcggaagagaaacgcgaaggctatgagcgcctgctgaaaatgcagaaccaacgtggcggtcgtgctctgttccaagacatcaagaaaccggcggaagatgagtggggtaaaaccccggatgcgatgaaggccgcaatggctttggagaagaaactgaatcaggcactgctggatctgcacgcgctgggttccgcacgtaccgacccgcacctgtgcgatttcttggaaacgcattttctggacgaagaggtcaagctgatcaagaaaatgggcgaccacctgacgaacttgcatcgtctgggtggtccagaggcgggtctgggtgagtacctgttcgagcgtctgactctgaagcatgatcccggg

SEQ ID NO: 57 - protein, 452 aa, Artificial Sequence
  An amino acid sequence of a fusion protein comprising a wild-type heavy chain human ferritin, GFP and a His tag
MGSHHHHHHSGENLYFQGGGGSGGGAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHDPG

SEQ ID NO: 58 - DNA, 1422 bp, Artificial Sequence
  A nucleotide sequence of a fusion protein comprising a wild-type heavy chain human ferritin, GFP, a His tag and a nucleating agent binding peptide
atgggcagccatcaccatcaccaccatagcggcgaaaacctgtactttcagggtggaggaggctctggtggaggcgccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgaccacggcgtctactagccaggtccgccaaaactatcatcaggacagcgaggcggcgatcaatcgccagattaacctggagttgtacgcaagctacgtttacctgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtatctttctgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccgggatgcacggtaaaacccaggcgacctctggtaccatccagtct

SEQ ID NO: 59 - protein, 474 aa, Artificial Sequence
  An amino acid sequence of a fusion protein comprising a wild-type heavy chain human ferritin, GFP, a His tag and a nucleating agent binding peptide
MGSHHHHHHSGENLYFQGGGGSGGGAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPGMHGKTQATSGTIQS

SEQ ID NO: 60 - DNA, 1398 bp, Artificial Sequence
  A nucleotide sequence of a fusion protein comprising a wild-type heavy chain human ferritin, GFP, a His tag and a nucleating agent
atgggcagccatcaccatcaccaccatagcggcgaaaacctgtactttcagggtggaggaggctctggtggaggcgccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgtctagccaaattcgccagaattacagcaccgacgttgaagcggcagtcaacagcctggttaatctgtacttgcaggccagctatacgtatctgagcctgggcttttactttgaccgcgacgatgtggccttggaaggcgtgagccactttttccgtgagctggcggaagagaaacgcgaaggctatgagcgcctgctgaaaatgcagaaccaacgtggcggtcgtgctctgttccaagacatcaagaaaccggcggaagatgagtggggtaaaaccccggatgcgatgaaggccgcaatggctttggagaagaaactgaatcaggcactgctggatctgcacgcgctgggttccgcacgtaccgacccgcacctgtgcgatttcttggaaacgcattttctggacgaagaggtcaagctgatcaagaaaatgggcgaccacctgacgaacttgcatcgtctgggtggtccagaggcgggtctgggtgagtacctgttcgagcgtctgactctgaagcatgatcccgggatgcacggtaaaacccaggcgacctctggtaccatccagtct

SEQ ID NO: 61 - protein, 466 aa, Artificial Sequence
  An amino acid sequence of a fusion protein comprising a wild-type heavy chain human ferritin, GFP, a His tag and a nucleating agent binding peptide
MGSHHHHHHSGENLYFQGGGGSGGGAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHDPGMHGKTQATSGTIQS

SEQ ID NO: 62 - DNA, 1782 bp, Artificial Sequence
  A nucleotide sequence of a fusion protein comprising a wild-type heavy chain human ferritin, GFP, a His tag and an antibody or an antigen binding fragment
atgggcagccatcaccatcaccaccatagcggcggtacgggcagcagcggtgccactgcaggtggtagcgataataaatttaacaaagaacagcaaaacgcgttttacgagattctgcacctgccgaatctgaatgaagagcagcgtaatgccttcatccagagcctgaaagatgatccgagccagagcgcgaacctgctggccgaagcgaaaaaactgaatgacgcgcaggccccgaaagtggacaacaaattcaataaagaacaacagaatgccttctacgagatcctgcatctgccgaacctgaatgaagaacagcgcaatgcctttatccagagcctgaaagatgatccgagccagagcgccaatctgctggccgaagccaaaaaactgaacgatgcgcaagcgccgaaagtgggcagcggcggtggtggaggaggctctggtggaggctggagccacccgcagttcgaaaaagccggcatgcgtaaaggcgaagaactgttcacgggcgtagtttcgattctggtcgagctggacggcgatgtgaacggtcataagtttagcgttcgcggtgaaggtgagggcgacgcgaccaacggcaaactgaccctgaagttcatctgcaccaccggcaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagggcggcagcagcggcggcagcggcaccggtatgaccacggcgtctactagccaggtccgccaaaactatcatcaggacagcgaggcggcgatcaatcgccagattaacctggagttgtacgcaagctacgtttacctgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtatctttctgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccggg

SEQ ID NO: 63 - protein, 594 aa, Artificial Sequence
  An amino acid sequence of a fusion protein comprising a wild-type heavy chain human ferritin, GFP, a His tag and an antibody or an antigen binding fragment
MGSHHHHHHSGGTGSSGATAGGSDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVGSGGGGGGSGGGWSHPQFEKAGMRKGEELFTGVVSILVELDGDVNGHKFSVRGEGEGDATNGKLTLKFICTTGKLPVPWPTLVTTLTYGVQCFARYPDHMKQHDFFKSAMPEGYVQERTISFKDDGYYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGSSGGSGTGMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPG

SEQ ID NO: 64 - DNA, 30 bp, Artificial Sequence
  A nucleotide sequence of a linker peptide
ggcggcagcagcggcggcagcggcaccggt

SEQ ID NO: 65 - DNA, 30 bp, Artificial Sequence
  A nucleotide sequence of a linker peptide
ggtggaggaggctctggtggaggcgccggc

SEQ ID NO: 66 - DNA, 48 bp, Artificial Sequence
  A nucleotide sequence of a linker peptide
ggcggcagcagcggcggcagcggcaccggtggagggggttgcaccggc

SEQ ID NO: 67 - DNA, 6 bp, Artificial Sequence
  A nucleotide sequence of a linker peptide
accgga

SEQ ID NO: 68 - DNA, 42 bp, Artificial Sequence
  A nucleotide sequence of a linker peptide
agcggcggtacgggcagcagcggtgccactgcaggtggtagc

SEQ ID NO: 69 - DNA, 54 bp, Artificial Sequence
  A nucleotide sequence of a linker peptide
ggctcgggctcgggctccggatctggttcaggttcaggatcgggctccgggtcc

SEQ ID NO: 70 - DNA, 63 bp, Artificial Sequence
  A nucleotide sequence of a linker peptide
ggctcggccgaagcggctgctaaagaagcagctgctaaagaggctgccgccaaggcagggtcc

SEQ ID NO: 71 - DNA, 63 bp, Artificial Sequence
  A nucleotide sequence of a linker peptide
ggctcgctgcttgagagccctaaagcattagaagaagcaccttggcctccaccagaagggtcc

SEQ ID NO: 72 - DNA, 1026 bp, Artificial Sequence
  A nucleotide sequence of a fusion protein lacking GFP but comprising a ferritin protein and a linker peptide
atgggcagccatcaccatcaccaccatagcggcggtacgggcagcagcggtgccactgcaggtggtagcgataataaatttaacaaagaacagcaaaacgcgttttacgagattctgcacctgccgaatctgaatgaagagcagcgtaatgccttcatccagagcctgaaagatgatccgagccagagcgcgaacctgctggccgaagcgaaaaaactgaatgacgcgcaggccccgaaagtggacaacaaattcaataaagaacaacagaatgccttctacgagatcctgcatctgccgaacctgaatgaagaacagcgcaatgcctttatccagagcctgaaagatgatccgagccagagcgccaatctgctggccgaagccaaaaaactgaacgatgcgcaagcgccgaaagtgggctcgggctcgggctccggatctggttcaggttcaggatcgggctccgggtccatgaccacggcgtctactagccaggtccgccaaaactatcatcaggacagcgaggcggcgatcaatcgccagattaacctggaggcgtacgcaagctacgtttacgcgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtgcgtttgcgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccggg

SEQ ID NO: 73 - protein, 342 aa, Artificial Sequence
  An amino acid sequence of a fusion protein lacking GFP but comprising a ferritin protein and a linker peptide
MGSHHHHHHSGGTGSSGATAGGSDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVGSGSGSGSGSGSGSGSGSMTTASTSQVRQNYHQDSEAAINRQINLEAYASYVYASMSYYFDRDDVALKNFAKYFLHQSHEEREHAEKLMKLQNQRGGRAFAQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPG

SEQ ID NO: 74 - DNA, 1035 bp, Artificial Sequence
  A nucleotide sequence of a fusion protein
atgggcagccatcaccatcaccaccatagcggcggtacgggcagcagcggtgccactgcaggtggtagcgataataaatttaacaaagaacagcaaaacgcgttttacgagattctgcacctgccgaatctgaatgaagagcagcgtaatgccttcatccagagcctgaaagatgatccgagccagagcgcgaacctgctggccgaagcgaaaaaactgaatgacgcgcaggccccgaaagtggacaacaaattcaataaagaacaacagaatgccttctacgagatcctgcatctgccgaacctgaatgaagaacagcgcaatgcctttatccagagcctgaaagatgatccgagccagagcgccaatctgctggccgaagccaaaaaactgaacgatgcgcaagcgccgaaagtgggctcggccgaagcggctgctaaagaagcagctgctaaagaggctgccgccaaggcagggtccatgaccacggcgtctactagccaggtccgccaaaactatcatcaggacagcgaggcggcgatcaatcgccagattaacctggaggcgtacgcaagctacgtttacgcgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtgcgtttgcgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccggg

SEQ ID NO: 75 - protein, 345 aa, Artificial Sequence
  An amino acid sequence of a fusion protein
MGSHHHHHHSGGTGSSGATAGGSDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVGSAEAAAKEAAAKEAAAKAGSMTTASTSQVRQNYHQDSEAAINRQINLEAYASYVYASMSYYFDRDDVALKNFAKYFLHQSHEEREHAEKLMKLQNQRGGRAFAQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPG

SEQ ID NO: 76 - DNA, 1035 bp, Artificial Sequence
  A nucleotide sequence of a fusion protein
atgggcagccatcaccatcaccaccatagcggcggtacgggcagcagcggtgccactgcaggtggtagcgataataaatttaacaaagaacagcaaaacgcgttttacgagattctgcacctgccgaatctgaatgaagagcagcgtaatgccttcatccagagcctgaaagatgatccgagccagagcgcgaacctgctggccgaagcgaaaaaactgaatgacgcgcaggccccgaaagtggacaacaaattcaataaagaacaacagaatgccttctacgagatcctgcatctgccgaacctgaatgaagaacagcgcaatgcctttatccagagcctgaaagatgatccgagccagagcgccaatctgctggccgaagccaaaaaactgaacgatgcgcaagcgccgaaagtgggctcgctgcttgagagccctaaagcattagaagaagcaccttggcctccaccagaagggtccatgaccacggcgtctactagccaggtccgccaaaactatcatcaggacagcgaggcggcgatcaatcgccagattaacctggaggcgtacgcaagctacgtttacgcgagcatgagctactatttcgatcgcgatgacgttgcgctgaaaaacttcgctaagtattttctgcaccaaagccacgaagaacgtgaacatgccgagaaactgatgaagctgcaaaatcagcgtggcggtcgtgcgtttgcgcaagatattaaaaagccggattgcgacgactgggaaagcggcctgaacgcaatggagtgtgcgctgcacttggagaaaaacgtgaatcagtccttgctggagctgcataagctggctaccgataagaatgatccgcacctgtgcgacttcattgaaacgcactatctgaatgaacaggtgaaggcaatcaaagaactgggtgatcacgtcaccaatctgcgtaaaatgggtgccccggagagcggcctggcggagtacctgtttgacaaacatacgttgggcgactcggacaacgagtctcccggg

SEQ ID NO: 77 - protein, 345 aa, Artificial Sequence
  An amino acid sequence of a fusion protein
MGSHHHHHHSGGTGSSGATAGGSDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVGSLLESPKALEEAPWPPPEGSMTTASTSQVRQNYHQDSEAAINRQINLEAYASYVYASMSYYFDRDDVALKNFAKYFLHQSHEEREHAEKLMKLQNQRGGRAFAQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESPG

SEQ ID NO: 78 - protein, 10 aa, Artificial Sequence
  An amino acid sequence of a linker peptide
GGSSGGSGTG

SEQ ID NO: 79 - protein, 10 aa, Artificial Sequence
  An amino acid sequence of a linker peptide
GGGGSGGGAG

SEQ ID NO: 80 - protein, 16 aa, Artificial Sequence
  An amino acid sequence of a linker peptide
GGSSGGSGTGGGGCTG

SEQ ID NO: 81 - protein, 2 aa, Artificial Sequence
  An amino acid sequence of a linker peptide
TG

SEQ ID NO: 82 - protein, 14 aa, Artificial Sequence
  An amino acid sequence of a linker peptide
SGGTGSSGATAGGS

SEQ ID NO: 83 - protein, 18 aa, Artificial Sequence
  An amino acid sequence of a linker peptide
GSGSGSGSGSGSGSGSGS

SEQ ID NO: 84 - protein, 21 aa, Artificial Sequence
  An amino acid sequence of a linker peptide
GSAEAAAKEAAAKEAAAKAGS

SEQ ID NO: 85 - protein, 21 aa, Artificial Sequence
  An amino acid sequence of a linker peptide
GSLLESPKALEEAPWPPPEGS

(19) United States
(12) Patent Application Publication
(10) Pub. No.: US 2019/0240282 A1
(43) Pub. Date: Aug. 8, 2019

(54) Nanocage

(71) Applicant: IMPERIAL INNOVATIONS LIMITED, London (GB)

(72) Inventors: Geoffrey Baldwin, London (GB); James Field, Cheam, Surrey (GB)

(73) Assignee: IMPERIAL INNOVATIONS LIMITED, London (GB)

(21) Appl. No.: 16/342,714

(22) PCT Filed: Oct. 19, 2017
(86) PCT No.: PCT/GB2017/053164

§ 371 Date: Apr. 17, 2019

(30) Foreign Application Priority Data
Oct. 20, 2016 (GB) 1617759.4

(51) Int. Cl. A61K 38/08; C07K 7/06; A61K 47/42; G01N 33/84; A61K 49/00; A61P 35/00; A61K 9/00

(52) U.S. Cl. CPC A61K 38/08; C07K 7/06; A61K 47/42; B82Y 5/00; A61K 49/00; A61P 35/00; A61K 9/0019; G01N 33/84

(57) ABSTRACT

The invention provides nanocages, and in particular to protein nanocages, and especially ferritin nanocages. The invention extends to variant ferritin polypeptides and their encoding nucleic acids, mutant ferritin nanocages, and their uses in diagnostics and drug delivery, as well as in phenotypic screens in drug development.

The present invention relates to nanocages, and in particular to protein nanocages, and especially ferritin nanocages. The invention extends to variant ferritin polypeptides and their encoding nucleic acids, mutant ferritin nanocages, and their uses in diagnostics and drug delivery, as well as in phenotypic screens in drug development.

Protein nanocages are a class of protein that self-assemble to form a three dimensional structure with a central cavity. A wide diversity of such proteins exist in nature with varying degrees of size, internal cavity dimensions and porosity. Ferritin is one such protein, it is found in all kingdoms of life and naturally acts to store iron and so protect the host from oxidative damage caused by the Fenton reaction. Ferritins have received a significant amount of attention for their potential bionanotechnology applications¹.

Recent studies have demonstrated the suitability and applicability of ferritin nanocages as potential agents for in vivo diagnostics and drug delivery. They have an external diameter of 12 nm and an internal cavity of 8 nm. It has been demonstrated that ferritin nanocages can be reversibly disassembled by a shift in pH² and this has been used to encapsulate the anti-cancer drug doxorubicin (Dox) at a ratio of approximately five Dox molecules per cage³. While this approach has been useful, it suffers from the problem of poor efficiency, as typically only 50% or less of fully assembled cages are recovered^{3, 4}. Furthermore, the proportion of the active agent, Dox, that can be encapsulated into the ferritin using a passive encapsulation technique, where the nanocage is reformed in the presence of the drug, is only around 0.1 to 0.4%³, which is very low and therefore wasteful in terms of drug loading.

Dox-loaded ferritin nanocages have successfully been used to demonstrate cancer targeting in mice models. Uchida and colleagues⁵ took the approach of encoding a peptide on the N-terminus of ferritin (Cys-Asp-Cys-Arg-Gly-Asp-Cys-Phe-Cys; RGD4C) a derivative of the RGD peptide known to target the αᵥβ₃ integrin, a tumour biomarker that is up-regulated on many types of tumour cells⁶⁻⁹. They demonstrated that these peptide-modified nanocages were able to bind to C32 melanoma cells⁵. Xie and colleagues subsequently used Dox-loaded RGD4C modified ferritin to successfully target and treat U87MG a tumour model in mice¹⁰. Further to this Yan and colleagues successfully demonstrated that Dox-loaded ferritin could be used to treat HT29 tumours in a mouse model⁴. In this latter study, they found that active targeting was not necessary and they proposed that uptake is via natural TfR1 receptor mediated endocytosis.

It has also been demonstrated that chimeric ferritin molecules can be made by linking different peptides to the N-terminus of the protein. By mixing the two types of ferritin in vitro and disassembling and reassembling using a pH switch, different peptides can be incorporated onto the same nanocage structure. This provides an interesting method by which multi-valent epitopes may be attached to the nanocage, but with limited control of the distribution¹¹.

Nanoparticles for the targeted delivery of drugs in vivo is an attractive idea that has been the subject of significant research. Nevertheless, over the last 10 years it has not been possible to significantly improve the targeting ratio of the designed nanoparticles¹². Most of the nanoparticles studied have been chemically based in a size range of 10-200 nm and rely on the enhanced permeability and retention effect (EPR) associated with many tumours. The poor delivery efficiency of these methods indicates that issues of biocompatibility and size are critical, with larger nanoparticles being readily sequestered by the mononuclear phagocytic system (MPS). In addition, the effectiveness of EPR is being questioned as a universal targeting mechanism. Improvements in drug targeting clearly need a change in biocompatibility, bioavailability and targeting efficiency.

Ferritin presents an attractive alternative to many chemical-based agents. It is large enough to be retained in the circulation (>8 nm), but is also biocompatible and non-immunogenic^{4, 11}. It is also small enough that it will have better tumour penetrating properties, since size (<50 nm) is an important factor in targeting efficiency¹². In addition, it has been proposed that invasion of ferritin to a tumour may occur via an intra-cell transport mechanism^{13, 14} and so will not be entirely dependent on the EPR effect for tumour invasion.

There is therefore a need for improved ferritin nanocages and components thereof, which can be used for targeted delivery of drugs to cells in vitro and in vivo and/or diagnosis, and in phenotypic screens in drug development.

To facilitate the numerous potential applications of a technology that can deliver drugs into cells, either in vivo or in vitro, the inventors set out to engineer a biocompatible platform that will facilitate a modular and generic approach. The inventors have developed a variant ferritin polypeptide in which the dimeric subunit interface has been mutated such that it is unable to self-assemble to form a nanocage structure. However, upon contacting the variant ferritin with a nucleating metallic core (such as a gold nanoparticle), the mutant self-assembles around the core, thereby forming a nanocage encapsulating the core. Furthermore, it is possible to encapsulate active agents, such as small molecule drugs, into the self-assembling nanocage structure, by attaching the active agent to the metal core prior to contacting it with the variant ferritin polypeptide. The invention thus provides a novel mechanism for the encapsulation of drugs into the ferritin nanocage without harsh denaturation conditions that are used in known systems. The inventors have also shown that the variant nanocage can be modified to be fluorescent by fusion of an N-terminal fluorescent protein to the mutant ferritin, for use in diagnostics and imaging experiments. Furthermore, they have also demonstrated that the nanocage can be specifically bound to antibodies or antigen-binding fragments thereof, and targeted to cells by further fusion of an antibody binding domain to the N-terminus of the variant ferritin, so that antibody-bound protein can specifically bind to target cells. The inventors also demonstrate that this antibody-based targeting platform can be used for the targeted delivery of drugs into cells, for example tumour cells.

Hence, in a first aspect of the invention, there is provided a variant ferritin polypeptide comprising a modified amino acid sequence of a wild-type ferritin polypeptide, the modified sequence being in a dimeric subunit interface or the N-terminus of the polypeptide, wherein the variant is incapable of assembling into a ferritin nanocage unless it is contacted with a nucleating agent.

Advantageously, the variant ferritin of the invention is biocompatible and not immunogenic. The inventors have engineered several embodiments of ferritin polypeptide monomers, which only self-assemble into a nanocage in the presence of a nucleating agent. These modified nanocage monomers can be used in diagnosis or in therapy, such as to facilitate the delivery of drugs into cells, either in vivo or in vitro.

In one preferred embodiment, the variant ferritin polypeptide comprises a modified bacterial ferritin, also known as bacterioferritin (Bfr). The bacterioferritin may be isolated from E. coli. It contains 24 subunits and 12 heme groups that bind between the dimeric protein interface. The nucleic acid (SEQ ID No:1) and amino acid (SEQ ID No:2) sequences of wild-type E. coli bacterioferritin are known, and may be represented herein as SEQ ID No:1 and SEQ ID No:2, or a fragment or variant thereof, as follows:

[SEQ ID No: 1 and 2]
ATG AAA GGT GAT ACT AAA GTT ATA AAT TAT CTC AAC AAA CTG TTG GGA AAT GAG CTT
M K G D T K V I N Y L N K L L G N E L
GTC GCA ATC AAT CAG TAC TTT CTC CAT GCC CGA ATG TTT AAA AAC TGG GGT CTC AAA CGT
V A I N Q Y F L H A R M F K N W G L K R
CTC AAT GAT GTG GAG TAT CAT GAA TCC ATT GAT GAG ATG AAA CAC GCC GAT CGT TAT ATT
L N D V E Y H E S I D E M K H A D R Y I
GAG CGC ATT CTT TTT CTG GAA GGT CTT CCA AAC TTA CAG GAC CTG GGC AAA CTG AAC ATT
E R I L F L E G L P N L Q D L G K L N I
GGT GAA GAT GTT GAG GAA ATG CTG CGT TCT GAT CTG GCA CTT GAG CTG GAT GGC GCG AAG
G E D V E E M L R S D L A L E L D G A K
AAT TTG CGT GAG GCA ATT GGT TAT GCC GAT AGC GTT CAT GAT TAC GTC AGC CGC GAT ATG
N L R E A I G Y A D S V H D Y V S R D M
ATG ATA GAA ATT TTG CGT GAT GAA GAA GGC CAT ATC GAC TGG CTG GAA ACG GAA CTT GAT
M I E I L R D E E G H I D W L E T E L D
CTG ATT CAG AAG ATG GGC CTG CAA AAT TAT CTG CAA GCA CAG ATC CGC GAA GAA GGT
L I Q K M G L Q N Y L Q A Q I R E E G

In one preferred embodiment, the variant bacterioferritin comprises a His tag. Preferably, the His tag is encoded by a nucleic acid sequence (SEQ ID No:3) or comprises an amino acid sequence (SEQ ID No:4), or a fragment of variant thereof, substantially as set out in SEQ ID No:3 and SEQ ID No:4, as follows:

[SEQ ID No: 3 and 4]

ATG CCC AGC CAT CAC CAT CAC CAC CAT AGC CCC
M G S H H H H H H S G

Preferably, the variant bacterioferritin comprises an N-terminal His tag. Accordingly, the variant bacterioferritin is preferably encoded by a nucleic acid (SEQ ID No:5) or comprises an amino acid (SEQ ID No:6) sequence, or fragment of variant thereof, substantially as set out in SEQ ID No: 5 and SEQ ID No:6, as follows:

[SEQ ID No: 5 and 6]
ATG GGC AGC CAT CAC CAT CAC CAC CAT AGC GGC GAA AAC CTG TAC TTT CAG ATG AAA
M G S H H H H H H S G E D L Y P Q M K
GGT GAT ACT AAA GTT ATA AAT TAT CTC AAC AAA CTG TTG GGA AAT GAG CTTGTC GCA
G D T K V I N Y L N K L L G N E L V A
ATC AAT CAG TAC TTT CTC CAT GCC CGA ATG TTT AAA AAC TGG GGT CTC AAA CGT CTC
I N Q Y F L H A R M F K N W G L K R L
AAT GAT GTG GAG TAT CAT GAA TCC ATT GAT GAG ATG AAA CAC GCC GAT CGT TAT ATT
N D V E Y H E S I D E M K H A D R Y I
GAG CGC ATT CTT TTT CTG GAA GGT CTT CCA AAC TTA CAG GAC CTG GGC AAA CTG AAC ATT
E R I L F L E G L P N L Q D L G K L N I
GGT GAA GAT GTT GAG GAA ATG CTG CGT TCT GAT CTG GCA CTT GAG CTG GAT GGC GCG AAG
G E D V E E M L R S D L A L E L D G A K
AAT TTG CGT GAG GCA ATT GGT TAT GCC GAT AGC GTT CAT GAT TAC GTC AGC CGC GAT ATG
N L R E A I G Y A D S V H D Y V S R D M
ATG ATA GAA ATT TTG CGT GAT GAA GAA GGC CAT ATC GAC TGG CTG GAA ACG GAA CTT GAT
M I E I L R D E E G H I D W L E T E L D
CTG ATT CAG AAG ATG GGC CTG CAA AAT TAT CTG CAA GCA CAG ATC CGC GAA GAA GGT
L I Q K M G L Q N Y L Q A Q I R E E G

In another preferred embodiment, the variant bacterioferritin comprises an amino acid sequence configured to bind a nucleating agent, and may for example be a silica binding peptide, or a metal binding peptide, such as gold, copper, iron. In an alternative embodiment, the variant may comprise a gadolinium binding peptide. Most preferably, however, the variant bacterioferritin comprises a gold-binding peptide. For example, a suitable metal binding peptide may be encoded by a nucleic acid sequence (SEQ ID No:7) or comprises an amino acid sequence (SEQ ID No:8), or a fragment of variant thereof, substantially as set out in SEQ ID No:7 and SEQ ID No:8, as follows:

[SEQ ID No: 7 and 8]
ATG CAC GGT AAA ACC CAG GCG ACC TCT GGT ACC ATC
M H G K T Q A T S G T I
CAG TCT
Q S

Preferably, the nucleating agent binding peptide is a C-terminal nucleating agent binding peptide. Accordingly, the variant bacterioferritin is preferably encoded by a nucleic acid sequence (SEQ ID No: 9) or comprises an amino acid sequence (SEQ ID No:10), or a fragment or variant thereof, substantially as set out in SEQ ID No:9 or SEQ ID No:10, as follows:

[SEQ ID No: 9 and 10]
ATG AAA GGT GAT ACT AAA GTT ATA AAT TAT CTC AAC AAA CTG TTG GGA AAT GAG CTT
M K G D T K V I N Y L N K L L G N E L
GTC GCA ATC AAT CAG TAC TTT CTC CAT GCC CGA ATG TTT AAA AAC TGG GGT CTC AAA CGT
V A I N Q Y F L H A R M F K N W G L K R
CTC AAT GAT GTG GAG TAT CAT GAA TCC ATT GAT GAG ATG AAA CAC GCC GAT CGT TAT ATT
L N D V E Y H E S I D E M K H A D R Y I
GAG CGC ATT CTT TTT CTG GAA GGT CTT CCA AAC TTA CAG GAC CTG GGC AAA CTG AAC ATT
E R I L F L E G L P N L Q D L G K L N I
GGT GAA GAT GTT GAG GAA ATG CTG CGT TCT GAT CTG GCA CTT GAG CTG GAT GGC GCG AAG
G E D V E E M L R S D L A L E L D G A K
AAT TTG CGT GAG GCA ATT GGT TAT GCC GAT AGC GTT CAT GAT TAC GTC AGC CGC GAT ATG
N L R E A I G Y A D S V H D Y V S R D M
ATG ATA GAA ATT TTG CGT GAT GAA GAA GGC CAT ATC GAC TGG CTG GAA ACG GAA CTT GAT
M I E I L R D E E G H I D W L E T E L D
CTG ATT CAG AAG ATG GGC CTG CAA AAT TAT CTG CAA GCA CAG ATC CGC GAA GAA GGT
L I Q K M G L Q N Y L Q A Q I R E E G
ACC GGA ATG CAC CGT AAA ACC CAC GCG ACC TCT CGT ACC ATC CAC TCT
T G M R G K T Q A T C G T T Q G

In another preferred embodiment, the variant bacterioferritin may comprise an N-terminal His tag and a C-terminal nucleating agent binding peptide. Preferably, therefore, the variant bacterioferritin is encoded by a nucleic acid sequence (SEQ ID No:11) or comprises an amino acid sequence (SEQ ID No:12), or a fragment or variant thereof, substantially as set out in SEQ ID No:11 or SEQ ID No:12, as follows:

[SEQ ID No: 11 and 12]
ATG GGC AGC CAT CAC CAT CAC CAC CAT AGC GGC GAA AAC CTG TAC TTT CAG ATG AAA
M G S H H H H H H S G E N L Y F Q M K
GGT GAT ACT AAA GTT ATA AAT TAT CTC AAC AAA CTG TTG GGA AAT GAG CTTGTC GCA
G D T K V I N Y L N K L L G N E L V A
ATC AAT CAG TAC TTT CTC CAT GCC CGA ATG TTT AAA AAC TGG GGT CTC AAA CGT CTC
I N Q Y F L H A R M F K N W G L K R L
AAT GAT GTG GAG TAT CAT GAA TCC ATT GAT GAG ATG AAA CAC GCC GAT CGT TAT ATT
N D V E Y H E S I D E M K H A D R Y I
GAG CGC ATT CTT TTT CTG GAA GGT CTT CCA AAC TTA CAG GAC CTG GGC AAA CTG AAC ATT
E R I L F L E G L P N L Q D L G K L N I
GGT GAA GAT GTT GAG GAA ATG CTG CGT TCT GAT CTG GCA CTT GAG CTG GAT GGC GCG AAG
G E D V E E M L R S D L A L E L D G A K
AAT TTG CGT GAG GCA ATT GGT TAT GCC GAT AGC GTT CAT GAT TAC GTC AGC CGC GAT ATG
N L R E A I G Y A D S V H D Y V S R D M
ATG ATA GAA ATT TTG CGT GAT GAA GAA GGC CAT ATC GAC TGG CTG GAA ACG GAA CTT GAT
M I E I L R D E E G H I D W L E T E L D
CTG ATT CAG AAG ATG GGC CTG CAA AAT TAT CTG CAA GCA CAG ATC CGC GAA GAA GGT
L I Q K M G L Q N Y L Q A Q I R E E G
ACC GGA ATG CAC GGT AAA ACC CAG GCG ACC TCT GGT ACC ATC CAG TCT
T G M K G K T Q A T S G T I Q S

As described in the Examples, the inventors were surprised to observe that the addition of the N-terminal His-tag meant that the bacterioferritin did not dimerise or purify in its nanocage composition, but instead as individual monomers. However, when the bacterioferritin had a C-terminal gold binding peptide, and after the addition of a gold nanoparticle nucleating agent, the variant bacterioferritin surprisingly formed a higher order structure consistent with a nanocage being formed around the gold nanoparticle. Surprisingly, the subtle modification of the bacterioferritin sequence with an N-terminal His tag has destabilised the nanocage structure of bacterioferritin under normal physiological conditions, and the use of a C-terminal metal binding peptide is sufficient to establish metal binding peptide-templated assembly of a nanocage without using harsh denaturation conditions.

In one preferred embodiment, the bacterioferritin is expressed in a bacterial host using a construct comprising a promoter, a ribosomal binding site (RBS) and nucleic acid encoding a His tag. The promoter used in the construct may be a compound promoter with the constitutive J23100 promoter in combination with the inducible T7 promoter. For example, the nucleic acid (SEQ ID No:13) and amino acid (SEQ ID No:14) sequences of a preferred bacterial expression construct may be represented herein as SEQ ID No:13 and SEQ ID No:14, respectively, or a fragment or variant thereof, as follows:

```
[SEQ ID No: 13 and 14]
                                  J23100 Promoter T7 Promoter
TTG ACG GCT AGC TCA GTC CTA GGT ACA GTG CTA GCT AAT ACG ACT CAC TAT AGG GAG ATA
                                RBS His Tag
CTA GAG AAA TCA AAT TAA GGA GGT AAG ATA ATG GGC AGC CAT CAC CAT CAC CAC CAT AGC GGC
                                                                                M G S H H H H H H S G
```

In a most preferred embodiment, however, the variant ferritin polypeptide comprises a modified mammalian ferritin, and most preferably modified human ferritin. Preferably, the variant human ferritin comprises one or more modification that disrupts the dimeric subunit interface of the wild-type human polypeptide, thereby rendering the variant incapable of forming heavy chain dimers unless it is contacted with a nucleating agent. Human ferritin may be composed of the light chain ferritin subunit (lFTN) or heavy chain ferritin subunit (hFTN), or a combination of both. By expressing either lFTN or hFTN in a host (e.g. E. coli), it is possible to create ferritin variant nanocages that consist of only a single protein monomer.

The nucleic acid (SEQ ID No:15) and amino acid (SEQ ID No:16) sequences of wild-type human heavy chain ferritin are known, and may be represented herein as SEQ ID No:15 and SEQ ID No:16, or a fragment or variant thereof, substantially as follows:

[SEQ ID No: 15 and 16]
ATG ACC ACG GCG TCT ACT AGC CAG GTC CGC CAA AAC TAT CAT CAG GAC AGC GAG
M T T A S T S Q V R Q N Y H Q D S E
GCG GCG ATC AAT CGC CAG ATT AAC CTG GAG TTG TAC GCA AGC TAC GTT TAC CTG
A A I N R Q I N L E L Y A S Y V Y L
AGC ATG AGC TAC TAT TTC GAT CGC GAT GAC GTT GCG CTG AAA AAC TTC GCT AAG
S M S Y Y F D R D D V A L K N F A K
TAT TTT CTG CAC CAA AGC CAC GAA GAA CGT GAA CAT GCC GAG AAA CTG ATG AAG
Y F L H Q S H E E R E H A E K L M K
CTG CAA AAT CAG CGT GGC GGT CGT ATC TTT CTG CAA GAT ATT AAA AAG CCG GAT
L Q N Q R G G R I F L Q D I K K P D
TGC GAC GAC TGG GAA AGC GGC CTG AAC GCA ATG GAG TGT GCG CTG CAC TTG GAG
C D D W E S G L N A M E C A L H L E
AAA AAC GTG AAT CAG TCC TTG CTG GAG CTG CAT AAG CTG GCT ACC GAT AAG AAT
K N V N Q S L L E L H K L A T D K N
GAT CCG CAC CTG TGC GAC TTC ATT GAA ACG CAC TAT CTG AAT GAA CAG GTG AAG
D P H L C D F I E T H Y L N E Q V K
GCA ATC AAA GAA CTG GGT GAT CAC GTC ACC AAT CTG CGT AAA ATG GGT GCC CCG
A I K E L G D H V T N L R K M G A P
GAG AGC GGC CTG GCG GAG TAC CTG TTT GAC AAA CAT ACG TTG GGC GAC TCG GAC
E S G L A E Y L F D K H T L G D S D
AAC GAG TCT CCC GGG
N E S P G

The nucleic acid (SEQ ID No:17) and amino acid (SEQ ID No:18) sequences of wild-type human light chain ferritin are known, and may be represented herein as SEQ ID No:17 and SEQ ID No:18, or a fragment or variant thereof, substantially as follows:

[SEQ ID No: 17 and 18]
ATG TCT AGC CAA ATT CGC CAG AAT TAC AGC ACC GAC GTT
M S S Q I R Q N Y S T D V
GAA GCG GCA GTC AAC AGC CTG GTT AAT CTG TAC TTG CAG GCC AGC TAT ACG TAT CTG AGC
E A A V N S L V N L Y L Q A S Y T Y L S
CTG GGC TTT TAC TTT GAC CGC GAC GAT GTG GCC TTG GAA GGC GTG AGC CAC TTT TTC CGT
L G F Y F D R D D V A L E G V S H F F R
GAG CTG GCG GAA GAG AAA CGC GAA GGC TAT GAG CGC CTG CTG AAA ATG CAG AAC CAA CGT
E L A E E K R E G Y E R L L K M Q N Q R
GGC GGT CGT GCT CTG TTC CAA GAC ATC AAG AAA CCG GCG GAA GAT GAG TGG GGT AAA ACC
G G R A L F Q D I K K P A E D E W G K T
CCG GAT GCG ATG AAG GCC GCA ATG GCT TTG GAG AAG AAA CTG AAT CAG GCA CTG CTG GAT
P D A M K A A M A L E K K L N Q A L L D
CTG CAC GCG CTG GGT TCC GCA CGT ACC GAC CCG CAC CTG TGC GAT TTC TTG GAA ACG CAT
L H A L G S A R T D P H L C D F L E T H
TTT CTG GAC GAA GAG GTC AAG CTG ATC AAG AAA ATG GGC GAC CAC CTG ACG AAC TTG CAT
F L D E E V K L I K K M G D H L T N L H
CGT CTG GGT GGT CCA GAG GCG GGT CTG GGT GAG TAC CTG TTC GAG CGT CTG ACT CTG AAG
R L G G P E A G L G E Y L F E R L T L K
CAT GAT CCC GGG
H D P G

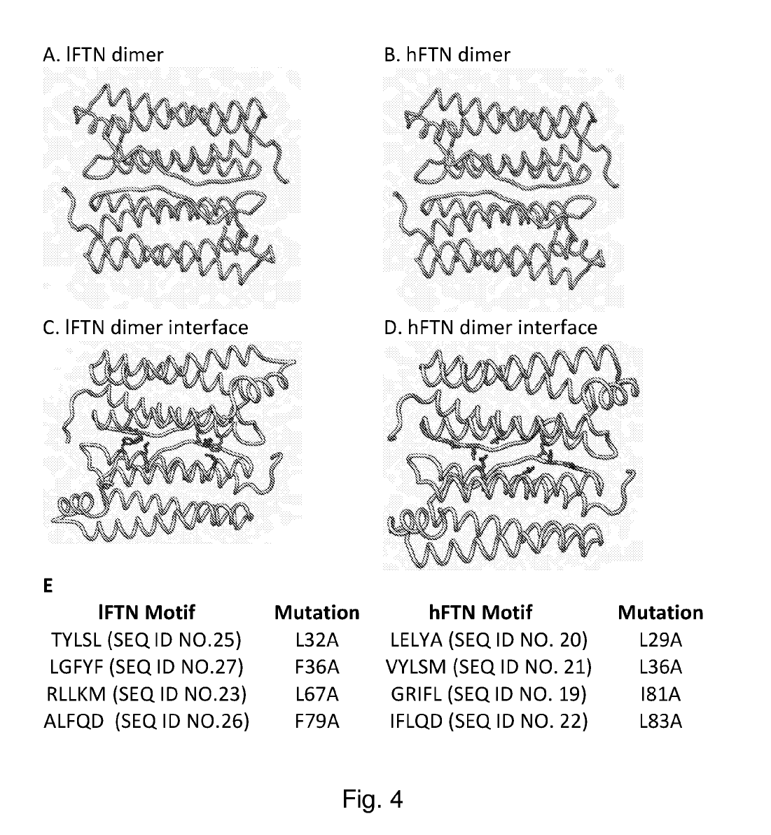

As described in the Examples, the inventors analysed over 147 conserved ferritin proteins, and managed to surprisingly identify several evolutionarily conserved domains at the dimeric interface of human ferritin proteins (heavy and light chains) that contain at least one hydrophobic residue (see Table 1 in Example 2). Hydrophobic residues within these conserved motifs were then carefully selected for site specific mutagenesis (see FIGS. 4C and 4D). Four mutations were created in the heavy chain variant of ferritin [hFTN (L29A L36A I81A L83A)] and four mutations were also made in the light chain variant of the polypeptide [lFTN (L32A F36A L67A F79A)] according to the conserved motifs that were identified.

Thus, in one preferred embodiment, the variant ferritin polypeptide comprises a variant human heavy chain ferritin. Preferably, the variant human heavy chain ferritin comprises one or more modification that disrupts the dimeric subunit interface of the wild-type polypeptide, thereby rendering the variant incapable of forming heavy chain dimers unless it is contacted with a nucleating agent.

Preferably, the variant human heavy chain ferritin comprises one or more modification in the wild-type polypeptide, wherein one or more hydrophobic residue in the heavy chain dimeric subunit interface of the polypeptide is substituted with a small amino acid residue, thereby rendering the variant incapable of forming heavy chain dimers, and hence higher order nanocages, unless it is contacted with a nucleating agent. Preferably, the heavy chain dimeric subunit interface comprises or consists of amino acid residues as set out in Table 1, i.e. SEQ ID No's: 19, 20, 21, 22 and 29.

Preferably, the variant heavy chain ferritin polypeptide comprises at least one modification in amino acids 29, 36, 81 or 83 of SEQ ID No:16. Preferably, the variant heavy chain ferritin polypeptide comprises at least two, more preferably at least three, and most preferably four modifications in amino acids 29, 36, 81 or 83 of SEQ ID No:16. Preferably, the variant heavy chain ferritin polypeptide is formed by modification of amino acid residue L29, L36, I81 and/or L83 of SEQ ID No:16. Preferably, the modification at amino acid L29 comprises a substitution with an alanine, i.e. L29A. Preferably, the modification at amino acid L36 comprises a substitution with an alanine, i.e. L36A. Preferably, the modification at amino acid I81 comprises a substitution with an alanine, i.e. I81A. Preferably, the modification at amino acid L83 comprises a substitution with an alanine, i.e. L83A.

Preferably, the variant human heavy chain ferritin polypeptide (L29A L36A I81A L83A) is encoded by a nucleic acid (SEQ ID No:30) or comprises an amino acid (SEQ ID No:31) sequence, or fragment of variant thereof, substantially as set out in SEQ ID No: 30 and SEQ ID No:31, as follows:

[SEQ ID No: 30 and 31]
ATG ACC ACG GCG TCT ACT AGC CAG GTC CGC CAA AAC TAT CAT CAG GAC AGC GAG
M T T A S T S Q V R Q N Y H Q D S E
GCG GCG ATC AAT CGC CAG ATT AAC CTG GAG GCG TAC GCA AGC TAC GTT TAC GCG
A A I N R Q I N L E A Y A S Y V Y A
AGC ATG AGC TAC TAT TTC GAT CGC GAT GAC GTT GCG CTG AAA AAC TTC GCT AAG
S M S Y Y F D R D D V A L K N F A K
TAT TTT CTG CAC CAA AGC CAC GAA GAA CGT GAA CAT GCC GAG AAA CTG ATG AAG
Y F L H Q S H E E R E H A E K L M K
CTG CAA AAT CAG CGT GGC GGT CGT GCG TTT GCG CAA GAT ATT AAA AAG CCG GAT
L Q N Q R G G R A F A Q D I K K P D
TGC GAC GAC TGG GAA AGC GGC CTG AAC GCA ATG GAG TGT GCG CTG CAC TTG GAG
C D D W E S G L N A M E C A L H L E
AAA AAC GTG AAT CAG TCC TTG CTG GAG CTG CAT AAG CTG GCT ACC GAT AAG AAT
K N V N Q S L L E L H K L A T D K N
GAT CCG CAC CTG TGC GAC TTC ATT GAA ACG CAC TAT CTG AAT GAA CAG GTG AAG
D P H L C D F I E T H Y L N E Q V K
GCA ATC AAA GAA CTG GGT GAT CAC GTC ACC AAT CTG CGT AAA ATG GGT GCC CCG
A I K E L G D H V T N L R K M G A P
GAG AGC GGC CTG GCG GAG TAC CTG TTT GAC AAA CAT ACG TTG GGC GAC TCG GAC
E S G L A E Y L F D K H T L G D S D
AAC GAG TCT CCC GGG
N E S P G

In an alternative preferred embodiment, the variant ferritin polypeptide comprises a variant human light chain ferritin. Preferably, the variant human light chain ferritin comprises one or more modification that disrupts the dimeric subunit interface of the wild-type polypeptide, thereby rendering the variant incapable of forming light chain dimers unless it is contacted with a nucleating agent. Preferably, the or each modification comprises substituting one or more hydrophobic residue in the light chain dimeric subunit interface of the polypeptide with a small amino acid residue, thereby rendering the variant incapable of forming light chain dimers and hence higher order nanocages, unless it is contacted with a nucleating agent. Preferably, the light chain dimeric subunit interface comprises or consists of amino acid residues as set out in Table 1, i.e. SEQ ID No's: 23, 24, 25, 26, 27, 28, and 29.

Preferably, the variant light chain ferritin polypeptide comprises at least one modification in amino acids 32, 36, 67 or 79 of SEQ ID No:18. Preferably, the variant light chain ferritin polypeptide comprises at least two, more preferably at least three, and most preferably four modifications in amino acids 32, 36, 67 or 79 of SEQ ID No:18. Preferably, the variant light chain ferritin polypeptide is formed by modification of amino acid residue L32, F36, L67 and/or F79 of SEQ ID No:18. Preferably, the modification at amino acid L32 comprises a substitution with an alanine, i.e. L32A. Preferably, the modification at amino acid F36 comprises a substitution with an alanine, i.e. F36A. Preferably, the modification at amino acid L67 comprises a substitution with an alanine, i.e. L67A. Preferably, the modification at amino acid F79 comprises a substitution with an alanine, i.e. F79A.

Preferably, the variant human light chain ferritin (L32A F36A L67A F79A) is encoded by a nucleic acid (SEQ ID No:32) or comprises an amino acid (SEQ ID No:33) sequence, or a fragment or variant thereof, substantially as set out in SEQ ID No: 32 and SEQ ID No:33, as follows:

[SEQ ID No: 32 and 33]
ATG TCT AGC CAA ATT CGC CAG AAT TAC AGC ACC GAC GTT
M S S Q I R Q N Y S T D V
GAA GCG GCA GTC AAC AGC CTG GTT AAT CTG TAC TTG CAG GCC AGC TAT ACG TAT GCG AGC
E A A V N S L V N L Y L Q A S Y T Y A S
CTG GGC GCG TAC TTT GAC CGC GAC GAT GTG GCC TTG GAA GGC GTG AGC CAC TTT TTC CGT
L G A Y F D R D D V A L E G V S H F F R
GAG CTG GCG GAA GAG AAA CGC GAA GGC TAT GAG CGC CTG GCG AAA ATG CAG AAC CAA CGT
E L A E E K R E G Y E R L A K M Q N Q R
GGC GGT CGT GCT CTG GCG CAA GAC ATC AAG AAA CCG GCG GAA GAT GAG TGG GGT AAA ACC
G G R A L A Q D I K K P A E D E W G K T
CCG GAT GCG ATG AAG GCC GCA ATG GCT TTG GAG AAG AAA CTG AAT CAG GCA CTG CTG GAT
P D A M K A A M A L E K K L N Q A L L D
CTG CAC GCG CTG GGT TCC GCA CGT ACC GAC CCG CAC CTG TGC GAT TTC TTG GAA ACG CAT
L H A L G S A R T D P H L C D F L E T H
TTT CTG GAC GAA GAG GTC AAG CTG ATC AAG AAA ATG GGC GAC CAC CTG ACG AAC TTG CAT
F L D E E V K L I K K M G D H L T N L H
CGT CTG GGT GGT CCA GAG GCG GGT CTG GGT GAG TAC CTG TTC GAG CGT CTG ACT CTG AAG
R L G G P E A G L G E Y L F E R L T L K
CAT GAT CCC GGG
H D P G

As described in the Examples, four mutations were created in the heavy [hFTN (L29A L36A I81A L83A)] and light [lFTN (L32A F36A L67A F79A)] chain variants of human ferritin. Each of these was constructed as N-terminal fusions with GFP (green fluorescent protein) to enable visualisation of the nanocage, either with or without a C-terminal gold binding peptide.

Hence, in one preferred embodiment, the variant ferritin, which may be bacterial ferritin or human ferritin (heavy or light chain), comprises a fluorophore, such as green fluorescent protein (GFP), red fluorescent protein (RFP) or cyan fluorescent protein (CFP). A preferred fluorophore comprises GFP, the nucleic acid (SEQ ID No:34) and amino acid (SEQ ID No:35) sequences of which are known, and are substantially as set out in SEQ ID No: 34 and SEQ ID No:35, as follows:

[SEQ ID No: 34 and 35]
ATG CGT AAA GGC GAA GAA CTG TTC ACG GGC GTA GTT TCG ATT CTG GTC GAG CTG
M R K G E E L F T G V V S I L V E L
GAC GGC GAT GTG AAC GGT CAT AAG TTT AGC GTT CGC GGT GAA GGT GAG GGC GAC
D G D V N G H K F S V R G E G E G D
GCG ACC AAC GGC AAA CTG ACC CTG AAG TTC ATC TGC ACC ACC GGC AAA CTG CCG
A T N G K L T L K F I C T T G K L P
GTG CCT TGG CCG ACC TTG GTG ACG ACG TTG ACG TAT GGC GTG CAG TGT TTT GCG
V P W P T L V T T L T Y G V Q C F A
CGT TAT CCG GAC CAC ATG AAA CAA CAC GAT TTC TTC AAA TCT GCG ATG CCG GAG
R Y P D H M K Q H D F F K S A M P E
GGT TAC GTC CAG GAG CGT ACC ATT TCC TTC AAG GAT GAT GGC TAC TAC AAA ACT
G Y V Q E R T I S F K D D G Y Y K T
CGC GCA GAG GTT AAG TTT GAA GGT GAC ACG CTG GTC AAT CGT ATC GAA TTG AAG
R A E V K F E G D T L V N R I E L K
GGT ATC GAC TTT AAA GAG GAT GGT AAC ATT CTG GGC CAT AAA CTG GAG TAT AAC
G I D F K E D G N I L G H K L E Y N
TTC AAC AGC CAT AAT GTT TAC ATT ACG GCA GAC AAG CAA AAG AAC GGC ATC AAG
F N S H N V Y I T A D K Q K N G I K
GCC AAT TTC AAG ATT CGC CAC AAT GTT GAG GAC GGT AGC GTC CAA CTG GCC GAC
A N F K I R H N V E D G S V Q L A D
CAT TAC CAG CAG AAC ACC CCA ATT GGT GAC GGT CCG GTT TTG CTG CCG GAT AAT
H Y Q Q N T P I G D G P V L L P D N
CAC TAT CTG AGC ACC CAA AGC GTG CTG AGC AAA GAT CCG AAC GAA AAA CGT GAT
H Y L S T Q S V L S K D P N E K R D
CAC ATG GTC CTG CTG GAA TTT GTG ACC GCT GCG GGC ATC ACC CAC GGT ATG GAC
H M V L L E F V T A A G I T H G M D
GAG CTG TAT AAG
E L Y K

The fluorophore is preferably disposed at or towards the N-terminus of the variant ferritin. Thus, preferably the variant human heavy chain ferritin is encoded by a nucleic acid (SEQ ID No:36) or comprises an amino acid (SEQ ID No:37) sequence, or a fragment of variant thereof, substantially as set out in SEQ ID No: 36 and SEQ ID No:37, as follows:

[SEQ ID No: 36 and 37]
ATC CGT AAA GGC GAA GAA CTC TTC ACG GGC GTA
M R K G E E L F T G V
GTT TCG ATT CTG GTC GAG CTG GAC GGC GAT GTG AAC GGT CAT AAG TTT AGC GTT CGC
V S I L V E L D G D V R G M K F S V R
GGT GAA GGT GAG GGC GAC GCG ACC AAC GGC AAA CTG ACC CTG AAG TTC ATC TGC ACC
G E G E G D A T M G K L T L K F I C T
ACC GGC AAA CTG CCG GTG CCT TGG CCG ACC TTG GTG ACG ACG TTG ACG TAT GGC GTG
T G K L P V P W P T L V I T L T Y G V
CAG TGT TTT GCG CGT TAT CCG GAC CAC ATG AAA CAA CAC GAT TTC TTC AAA TCT GCG
Q C F A R Y P D H M K Q R D F P K S A
ATG CCG TAG GGT TAC GTC CAG GAG CGT ACC ATT TCC TTC AAG GAT GAT GGC TAC TAC
M P E G Y V Q E R T I S F K D D G Y Y
AAA ACT CGC GCA GAG GTT AAG TTT GAA GGT GAC ACG CTG GTC AAT CGT ATC GAA TTG
K T P A E V K F E G D T L V H P I E L
AAG GGT ATC GAC TTT AAA GAG GAT CGT AAC ATT CTG CGC CAT AAA CTG CAG TAT AAC
K G I D F K E D G N I L S A K L E Y H
TTC AAC AGC CAT AAT GTT TAC ATT ACG GCA GAC AAG CAA AAG AAC GGC ATC AAG GCC
F R S K N V Y I T A D K Q K M G I K A
AAT TTC AAG ATT CGC CAC AAT GTT GAG GAC GGT AGC GTC CAA CTG GCC GAC CAT TAC
N F K I R A M V E D G S V Q L A D A Y
CAG CAG AAC ACC CCA ATT GGT GAC GGT CCG GTT TTG CTG CCG GAT AAT CAC TAT CTG
Q Q M T P I G D G P V L L P D N H T L
AGC ACC CAA AGC GTG CTG AGC AAA GAT CCG AAC GAA AAA CGT GAT CAC ATG GTC CTG
S T Q S V L S K D P N E K P D H M V L
CTG GAA TTT GTG ACC GCT GCG GGC ATC ACC CAC GGT ATG GAC GAG CTG TAT AAG GGC
L E F V T A A G I T H G M D E L Y K G
GGC AGC AGC GGC GGC AGC GGC ACC GGT ATG ACC ACG GCG TGT AGT AGC CAG GTC CGC
G S S G G S G T G M T T A S T S Q V R
CAA AAC TAT CAT CAG GAC AGC GAG GCG GCG ATC AAT CGC CAG ATT AAC CTG GAG gcg
Q N Y R Q D S E A A I N E Q I N L E A
TAC GCA AGC TAC GTT TAC gcg AGC ATC AGC TAC TAT TTC TAT CGC GAT GAC GTT GCG
Y A S Y V Y A D M S Y Y F D R D D V A
CTG AAA AAC TTC GCT AAG TAT TTT CTG CAC CAA AGC CAC GAA GAA CGT GAA CAT GCC
L K N F A K Y F L M Q S R E E R E M A
GAG AAA CTG ATG AAG CTG CAA AAT CAG CGT GGC GGT CGT gcg TTT gcg CAA GAT ATT
E K L M E L G R Q R G G P A F A Q D I
AAA AAG CCG GAT TGC GAC GAC TGG GAA AGC GGC CTG AAC GCA ATG GAG TGT GCG ATG
K K P D C D D W E E G L N A M E C A L
CAC TTG GAG AAA AAC GTG AAT CAG TGC TTG CTG GAG CTG CAT AAG CTG GCT ACC GAT
K L E K N V N Q S L L E L N K L A T D
AAG AAT GAT CCG CAC CTG TGC GAC TTC ATT GAA ACG CAC TAT CTG AAT GAA CAG GTG
K G D F A L C D F I E T A Y L N E Q V
AAG GCA ATC AAA GAA CTG GGT GAT CAC GTC ACC AAT CTG CGT AAA ATG GGT GCC CGG
K A I K E L G D H V T N L P K M G A P
GAC AGC CGC CTG GCG GAG TAC CTG TTT GAC AAA CAT ACG TTG CGC GAC TCG GAC AAC
E S G L A E Y L F D K A T L G D S D N
GAG TCT CCC GCG
E S P G

Preferably, the variant human light chain ferritin is encoded by a nucleic acid (SEQ ID No:38) or comprises an amino acid (SEQ ID No:39) sequence, or fragment of variant thereof, substantially as set out in SEQ ID No: 38 and SEQ ID No:39, as follows:

[SEQ ID No: 38 and 39]
ATG CGT AAA CGC GAA GAA CTG TTC ACG CGC GTA
M P K G E E L P I G V
GTT TCG ATT CTG GTC GAG CTG GAC GGC GAT GTG AAC CGT CAT AAG TTT AGC GTT CGC
V S I L V E L D G D V D G E F F S V P
GCT GAA GCT GAG GGC GAC GCG ACC AAC GGC AAA CTG ACC CTG AAG TTC ATC TGC ACC
G E G E G D A T R G K L T L K F T C T
ACC GGC AAA CTG CCG GTG CCT TGG CCG ACC TTG GTG ACG ACG TTG ACG TAT GGC GTG
T G K L P V P W P T L V T T L T Y G V
CAG TGT TTT GCG CGT TAT CCG GAC CAC ATG AAA CAA CAC GAT TTC TTC AAA TCT GCG
Q C F A R Y P D K M K Q K D F F K S A
ATG CCG GAG GGT TAC GTC CAG GAG CGT ACC ATT TCC TTC AAG GAT GAT GGC TAC TAC
M P E G Y V Q E R T I S F K D D G Y Y
AAA ACT CGC GCA GAG GTT AAG TTT GAA GGT GAC ACG CTG GTC AAT CGT ATC GAA TTG
K T P A E V K F E G D I L V D P I E L
AAG CGT ATC GAC TTT AAA GAG GAT CGT AAC ATT CTG GGC CAT AAA CTG GAG TAT AAC
F G I D F E E D G D I L G E E L E I D
TTC AAC AGC CAT AAT GTT TAC ATT ACG GCA GAC AAG CAA AAG AAC GGC ATC AAG GCC
F R S A R V T I T A D K Q K R G I K A
AAT TTC AAG ATT CGC CAC AAT GTT GAG GAC GGT AGC GTG CAA CTG GCC GAC CAT TAC
N F K I R R N V E L G S V W L A D A Y
CAG CAG AAC ACC CCA ATT GGT GAC GGT CCG GTT TTG CTG CCG GAT AAT CAC TAT CTG
Q Q N T P I G D G P V L L P D N K Y L
AGC ACC CAA AGC GTG CTG AGC AAA GAT CCG AAC GAA AAA CGT GAT CAC ATG GTC CTG
S T Q S V L S K D P N E K R D N M V L
CTG GAA TTT CTG ACC GCT GCC CGC ATC ACC CAC GGT ATG GAC GAG CTG TAT AAG GGC
L E F V T A A G I T H G M D E L Y K G
GGC AGC AGC GGC GGC AGC GGC ACC GGT ATG TCT AGC CAA ATT CGC CAG AAT TAC AGC
G S S G G S G T G M S S Q I R Q N Y S
ACC GAC GTT GAA GCG GCA GTC AAC AGC CTG GTT AAT CTG TAC TTG CAG GCC AGC TAT
T D V E A A V N S L V N L Y L Q A S Y
ACG TAT GCG AGC CTG GGC GCG TAC TTT GAC CGC GAC GAT GTG GCC TTG GAA GGC GTG
T Y A S L G A Y F D R D D V A L E G V
AGC CAC TTT TTC CGT GAG CTG GCG GAA GAG AAA CGC GAA GGC TAT GAG CGC CTG GCG
S H F F R E L A E E K R E G Y E R L A
AAA ATG CAG AAC CAA CGT GGC GGT CGT GCT CTG GCG CAA GAC ATC AAG AAA CCG GCG
K M Q N Q R G G R A L A Q D I K K P A
GAA GAT GAG TGG GGT AAA ACC CCG GAT GCG ATG AAG GCC GCA ATG GCT TTG GAG AAG
E D E W G K T P D A M K A A M A L E K
AAA CTG AAT CAG GCA CTG CTG GAT CTG CAC GCG CTG GGT TCC GCA CGT ACC GAC CCG
K L N Q A L L D L H A L G S A R T D P
CAC CTG TGC GAT TTC TTG GAA ACG CAT TTT CTG GAC GAA GAG GTC AAG CTG ATC AAG
H L C D F L E T H F L D E E V K L I K
AAA ATG GGC GAC CAC CTG ACG AAC TTG CAT CGT CTG GGT GGT CCA GAG GCG GGT CTG
K M G D H L T N L H R L G G P E A G L
GGT GAG TAC CTG TTC GAG CGT CTG ACT CTG AAG CAT GAT CCC GGG
G E Y L F E R L T L K H D P G

Preferably, the variant human heavy or light chain ferritin comprises a His tag, more preferably an N-terminal His tag. Preferably, the His tag is encoded by a nucleic acid sequence (SEQ ID No:3) or comprises an amino acid sequence (SEQ ID No:4), or a fragment of variant thereof, as disclosed herein.

Hence, preferably the variant human heavy chain ferritin is encoded by a nucleic acid (SEQ ID No:40) or comprises an amino acid (SEQ ID No:41) sequence, or a fragment of variant thereof, substantially as set out in SEQ ID No: 40 and SEQ ID No:41, as follows:

[SEQ ID No: 40 and 41]
ATG GGC AGC CAT CAC CAT CAC CAC CAT AGC GGC GAA AAC CTG TAC TTT CAG GGT GGA
M G S H H H H H H S G E N L Y F Q G G
GGA GGC TCT GGT GGA GGC GCC GGC ATG CGT AAA GGC GAA GAA CTG TTC ACG GGC GTA
G G S G G G A G M A K G E E L F T G V
GTT TCG ATT CTG GTC GAG CTG GAC GGC GAT GTG AAC GGT CAT AAG TTT AGC GTT CGC
V S I L V E L D G D V N G R K F S V P
GGT GAA GGT GAG GGC GAC GCG ACC AAC GGC AAA CTG ACC CTG AAG TTG ATC TGC ACC
G E G E G D A T N G K L T L K F T C T
ACC GGC AAA CTG CCG GTG CCT TGG CCG ACC TTG GTG ACG ACG TTG ACG TAT GGC GTG
T G K L P V P Q P I L V T T L T Y G V
CAG TGT TTT GCG CGT TAT CCG GAC CAC ATG AAA CAA CAC GAT TTC TTC AAA TCT GCG
Q C F A P Y P D H Q K Q H D F F K G A
ATG CCG CAG GGT TAC GTC CAG GAG GGT ACC ATT TCC TTC AAG CAT GAT GGC TAC TAC
M P E G Y V Q E P T T S P K D D G Y Y
AAA ACT CGC GCA GAG GTT AAG TTT GAA GGT GAC ACG CTG GTC AAT CGT ATC GAA TTG
K T R A E V K F E G D T L V N R I E L
AAG GGT ATC GAC TTT AAA GAG GAT GGT AAC ATT CTG GGC CAT AAA CTG GAG TAT AAC
K G I D P K E D G N I L G H K L B Y N
TTC AAC AGC CAT AAT GTT TAC ATT ACG GCA GAC AAG CAA AAG AAC GGC ATC AAG GCC
F N S H N V Y T T A C K Q K N G T K A
AAT TTC AAG ATT CGC CAC AAT GTT CAG GAC GGT AGC GTC CAA CTG GCC GAC CAT TAC
N F E T R A N V E D G G V Q L A D A Y
CAG CAG AAC ACC CCA ATT GGT GAC GGT CCG GTT TTG CTG CCG GAT AAT CAC TAT CTG
Q Q N T P T G D G P V L L F D N H T L
AGC ACC CAA AGC GTG CTG AGC AAA GAT CCG AAC GAA AAA CGT GAT CAC ATG GTC CTG
S T Q S V L G K D P N E K P D W M V L
CTG GAA TTT GTG ACC GCT GCG GGC ATC ACC CAC GGT ATG GAC GAG CTG TAT AAG GGC
L E P V T A A G T T H G M D E L Y K G
GGC AGC AGC GGC GGC AGC GGC ACC GGT ATG ACC ACG GCG TCT ACT AGC CAG GTC CGC
G S S G G S G T G M T T A S T K G V R
CAA AAC TAT CAT CAG GAC AGC GAG GCG GCG ATC AAT CGC CAG ATT AAC CTG GAG TCG
Q N Y A Q D S E A A T N E Q T N L E A
TAC GCA AGC TAC GTT TAC gag AGC ATG AGC TAC TAT TTC GAT CGC GAT GAC GTT CGG
Y A S Y V Y A S M G Y Y F D R D D V A
CTG AAA AAC TTG GCT AAG TAT TTT CTG CAC CAA AGC CAC CAA CAA CGT GAA CAT CGC
L K N F A K Y F L H Q S H E E R E H A
GAG AAA CTG ATG AAG CTG CAA AAT CAG CGT GGC GGT CGT gcg TTT gcg CAA GAT ATT
E K L M E L Q N Q R G G P A F A Q D I
AAA AAG CCG GAT TGC GAC GAC TGG CAA ACC GGC CTG AAC GCA ATG GAG TGT GCG CTG
K K P D C D D W E S G L N A M E C A L
CAC TTG GAG AAA AAC GTG AAT CAG TCC TTG CTG GAG CTG CAT AAC CTG GCT ACC GAT
H L E K N V H Q S L L E L A E L A T D
AAG AAT GAT CCG CAC CTG TGC GAC TTC ATT GAA ACG CAC TAT CTG AAT GAA CAG GTG
K R D P H L C D F I E T H Y L N E Q V
AAG GCA ATC AAA GAA CTG GGT GAT CAC GTC ACC AAT CTG CGT AAA ATG GGT GCC CCG
K A I Y E L G D H V T N L R E M G A P
GAG AGC GGC CTG GCG GAG TAC CTG TTT GAC AAA CAT ACG TTG GGC GAC TCG GAC AAC
E S G L A E Y L F D E W T L G D S D H
GAG TCT CGC GGG
E S P G

Hence, preferably the variant human light chain ferritin is encoded by a nucleic acid (SEQ ID No:42) or comprises an amino acid (SEQ ID No:43) sequence, or a fragment of variant thereof, substantially as set out in SEQ ID No: 42 and SEQ ID No:43, as follows:

[SEQ ID No: 42 and 43]
ATG GGC AGC CAT CAC CAT CAC CAC CAT AGC GGC GAA AAC CTG TAG TTT CAG GGT GGA
M G S H H H H H H S G E N L Y F Q G G
GGA GGC TCT GGT GGA GGC GCC GGC ATG CGT AAA GGC GAA GAA GTG TTG ACG GGC GTA
G G S G G G A G M A K G E E L F T G V
GTT TCG ATT CTG GTC GAG CTG GAC GGC GAT GTG AAC GCT CAT AAG TTT ACC GTT CGC
V E I L V E L D G D V N G E K F S V R
GGT GAA GGT GAG GGC GAC GCG ACC AAC GGC GAA CTG ACC CTG AAG TTC ATC TGC AGC
G E G E G D A T H G K L T L K F T G T
AGC GGC AAA CTG CGG GTG CGT TGG CGG AGC TTG CTG ACG ACG TTG ACG TAT GGC GTG
T G K L P V P W P T L V T T L T Y G V
CAG TGT TTT GCG CGT TAT GCG GAC CAC ATG AAA CAA CAC GAT TTC TTC AAA TCT GCG
Q C F A K Y P D A M K Q R D F F K S A
ATG GCG GAG GGT TAG GTG CAG GAG GGT ACC ATT TCC TTG AAG GAT GAT GGC TAG TAG
M F E G Y V Q E K T I S F K D D G Y Y
AAA ACT CGC GCA GAG GTT AAG TTT GAA GCT GAC ACG CTG GTC AAT CCT ATC GAA TTG
K T R A E V K F E G D T L V N R I E L
AAG GGT ATC GAC TTT AAA GAG GAT GGT AAC ATT CTG GGC CAT AAA CTG GAG TAT AAC
K G I D F K E D G N T L G H K L E Y N
TTC AAC AGC CAT AAT GTT TAC ATT ACG GCA GAC AGG CAA AGG AGC GGC ATC AAC GGC
F D S E D V Y T T A D F Q F D G T F A
AAT TTC AAC ATT CGC CAC AAT GTT CAC CAC GGT AGC CTC CAA CTC GCC CAC CAT TAC
D F E T R R N C E D G S V Q L A D R Y
CAG GAG AAC ACC CCA ATT GGT GAC GGT GCG GTT TTG CTG CCG GAT AAT CAC TAT CTG
Q Q N T P T G D G P V L L P D N E Y L
AGC ACG GAA AGG GTG GTG AGG AAA GAT GCG AAG GAA AAA GCT GAT CAG ATG GTC CTG
S T Q S V L S K D P N E K E D K M V L
CTG GAA TTT GTG ACC GCT GCG GCC ATC ACC CAC CGT ATG GAC GAG CTG TAT AAG GGC
L E F V T A A G I T H G M D E L Y K G
GGC AGC AGC GGC GGC AGC GGC ACC GGT ATG TCT AGC CAA ATT CGC CAG AAT TAC AGC
G S S G G S G T G M S S Q I R Q N Y S
ACC GAC GTT GAA GCG GCA GTC AAC AGC CTG GTT AAT CTG TAC TTG CAG GCC AGC TAT
T D V E A A V N S L V N L Y L Q A S Y
ACG TAT GCG AGC CTG GGC GCG TAC TTT GAC CGC GAC GAT GTG GCC TTG GAA GGC GTG
T Y A S L G A Y F D R D D V A L E G V
AGC CAC TTT TTC CGT GAG CTG GCG GAA GAG AAA CGC GAA GGC TAT GAG CGC CTG GCG
S H F F R E L A E E K R E G Y E R L A
AAA ATG CAG AAC CAA CGT GGC GGT CGT GCT CTG GCG CAA GAC ATC AAG AAA CCG GCG
K M Q N Q R G G R A L A Q D I K K P A
GAA GAT GAG TGG GGT AAA ACC CCG GAT GCG ATG AAG GCC GCA ATG GCT TTG GAG AAG
E D E W G K T P D A M K A A M A L E K
AAA CTG AAT CAG GCA CTG CTG GAT CTG CAC GCG CTG GGT TCC GCA CGT ACC GAC CCG
K L N Q A L L D L H A L G S A R T D P
CAC CTG TGC GAT TTC TTG GAA ACG CAT TTT CTG GAC GAA GAG GTC AAG CTG ATC AAG
H L C D F L E T H F L D E E V K L I K
AAA ATG GGC GAC CAC CTG ACG AAC TTG CAT CGT CTG GGT GGT CCA GAG GCG GGT CTG
K M G D H L T N L H R L G G P E A G L
GGT GAG TAC CTG TTC GAG CGT CTG ACT CTG AAG CAT GAT CCC GGG
G E Y L F E R L T L K H D P G

The skilled person would appreciate how to construct a variant bacterioferritin polypeptide comprising a fluorophore, preferably GFP (SEQ ID No:34 and 35), at the N-terminus of the modified ferritin (SEQ ID No:5, 6, 9, 10, 11 or 12).

In another preferred embodiment, the variant human heavy or light chain ferritin comprises a nucleating agent binding peptide, for example a silica binding peptide, or a metal binding peptide, such as gold, copper, iron, or it may be a gadolinium binding peptide. Most preferably, the variant human heavy or light chain ferritin comprises a gold-binding peptide. For example, a suitable metal binding peptide may comprise or consist of an amino acid sequence substantially as set out in SEQ ID No:8, or a fragment of variant thereof, or encoded by a nucleic acid sequence substantially as set out in SEQ ID No: 7. Preferably, the nucleating agent binding peptide is a C-terminal nucleating agent binding peptide.

With the human ferritin, modification of the dimerization interface was required to prevent cage formation, and a nanocage was surprisingly formed with gold nanoparticles even in the absence of a C-terminal gold binding peptide. In another preferred embodiment, the variant human heavy or light chain ferritin comprises an N-terminal His tag and a C-terminal nucleating agent binding peptide.

Accordingly, preferably the variant human heavy chain ferritin is encoded by a nucleic acid (SEQ ID No:44) or comprises an amino acid (SEQ ID No:45) sequence, or a fragment or variant thereof, substantially as set out in SEQ ID No: 44 and SEQ ID No:45, as follows:

[SEQ ID No: 44 and 45]
ATG GGC AGC CAT CAC CAT CAC CAC CAT AGC GGC GAA AAC CTG TAC TTT CAG GGT GGA
M G S R B Y B R B A G E N L Y F Q G G
GGA GGC TCT GGT GGA GGC GCC GGC ATG CGT AAA GGC GAA GAA CTG TTC ACG GGC GTA
G G S G G G A G M K K G E E L P T G V
GTT TCG ATT CTG GTC GAG CTG GAC GGC GAT GTG AAC GGT CAT AAG TTT AGC GTT CGC
V S I L V E L D G D V Q G R K P S V R
GGT GAA GGT GAG GGC GAC GCG ACC AAC GGC AAA CTG ACC CTG AAG TTC ATC TGC ACC
G E G E G D A T N G K L T L K F I C T
ACC GGC AAA CTG CCG GTG CCT TGG CCG ACC TTG GTG ACG ACG TTG ACG TAT GGC GTG
T G K L F V P W P T L V T T L T Y G V
CAG TGT TTT GCG CGT TAT CCG GAC CAC ATG AAA CAA CAC GAT TTC TTC AAA TCT GCG
Q Q P A R Y P D H M E Q H D P F K S A
ATG CCG GAG GGT TAC TGC CAG GAG CGT ACC ATT TCC TTC AAG GAT GAT GGC TAC TAC
M P B G Y V Q E R T L S F K D D G Y Y
AAA ACT CGC GCA GAG GTT AAG TTT GAA GGT GAC ACG CTG GTC AAT CGT ATC GAA TTG
K T P A E V K F E G D T L V M K I D L
AAG GGT ATC GAC TTT AAA GAG GAT GGT AAC ATT CTG GGC CAT AAA CTG GAG TAT AAC
K G I D F K S D G H I L G H K L E Y N
TTC AAC AGC CAT AAT GTT TAC ATT ACG GCA GAC AAG CAA AAG AAC GGC ATC AAG GCC
F N S R M V Y I Y A D K Q K N G I K A
AAT TTC AAG ATT CGC CAC AAT GTT GAG GAC GGT AGC GTC CAA CTG GCC GAC CAT TAC
N F K Y R A N V S D G S V Q L A D R Y
CAG CAG AAC ACC CCA ATT GGT GAC GGT CCG GTT TTG CTG CCG GAT AAT CAC TAT CTG
Q Q N Y P Y G D G P V L L P D G K Y L
AGC ACC CAA AGC GTG CTG AGC AAA GAT CCG AAC GAA AAA CGT GAT CAC ATG GTC CTG
S T Q S V L S K D P R S K R D K M V L
CTG GAA TTT GTG ACC GCT GCG GGC ATC ACC CAC GGT ATG GAC GAG CTG TAT AAG GGC
L E P V T A A G T T H G N D E L Y K G
GGC AGC AGC GGC GGC AGC GGC ACC GGT ATG ACC ACG GCG TCT ACT AGC CAG GTC CGC
G S S G G S G T G M T T A S T S Q Y E
CAA AAC TAT CAT CAG GAC AGC GAG GCG GCG ATC AAT CGC CAG ATT AAC CTG GAG gcg
Q R Y M Q D S E A A I N R Q I N L E A
TAC GCA AGC TAC GTT TAC gcg AGC ATG AGC TAC TAT TTC GAT CGC GAT GAC GTT GCG
Y A S Y V Y A S M S Y Y F D R D D V A
CTG AAA AAC TTC GCT AAG TAT TTT CTG CAC CAA AGC CAC GAA GAA CGT GAA CAT GCC
L K N F A K Y F L H Q S R D S K E R A
GAG AAA CTG ATG AAG CTG CAA AAT CAG CGT GGC GGT CGT gcg TTT gcg CAA GAT ATT
E K L M K L Q N Q P G C K A F A Q D A
AAA AAG CCG GAT TGC GAC GAC TGG GAA AGC GGC CTG AAC GCA ATG GAG TGT GCG CTG
K K P D C D D Q E S G L R A M E C A L
CAC TTG CAG AAA AAC GTG AAT CAG TCC TTG CTG GAG CAG CAT AAG CTG GCT ACC GAT
H L E K R V N Q S L L E L R K L A I D
AAG AAT CAT CCG CAC CTG TGC GAG TTC ATA CAA ACG CAC TAT CTG AAT GAA CAG CTG
K N L P R L C D P I E T R Y L R E Q V
AAG GCA ATC AAA GAA CTG GGT GAT CAC GTC ACC AAT CTG CGT AAA GTG GGT GCC CCG
K A I K S L G D R V T R L K K M G A P
GAG AGC GGC CTG GGG GAG TAC CTG TTT GAC AAA CAT ACG TTG GGC GAC TCG GAC AAC
E S G L A E Y L F D K R T L G D S D N
GAG TCT CCC GGG ATG CAC GGT AAA ACC CAG GCG ACC TCT GGT ACC ATC CAG TCT
E S P G M R G K T Q A I S G T Y Q S

Preferably, the variant human light chain ferritin is encoded by a nucleic acid (SEQ ID No:46) or comprises an amino acid (SEQ ID No:47) sequence, or fragment or variant thereof, substantially as set out in SEQ ID No: 46 and SEQ ID No:47, as follows:

[SEQ ID No: 46 and 47]
ATG GGC AGC CAT CAC CAT CAC CAC CAT AGC GGC GAA AAC CTG TAC TTT CAG GGT GGA
M G S H H H H H H S G E N L Y F Q G G
GGA GGC TCT GGT GGA GGC GCC GGC ATG CGT AAA GGC GAA GAA CTG TTC ACG GGC GTA
G G S G G G A G M R K G E E L P T G Y
GTT TCG ATT CTG GTC GAG CTG GAC GGC GAT GTG AAC GGT CAT AAG TTT AGC GTT CGC
V S I L V E L D G D V N G R K F S V R
GGT GAA GGT GAG GGC GAC GCG ACC AAC GGC AAA CTG ACC CTG AAG TTG ATC TGC ACC
G E G E G D A T N G K L T L K F T C T
ACC GGC AAA CTG CCG GTG CCT TGG CCG ACC TTG GTG ACG ACG TTG ACG TAT GGC GTG
T G K L P V P Q P I L V T T L T Y G V
CAG TGT TTT GCG CGT TAT CCG GAC CAC ATG AAA CAA CAC GAT TTC TTC AAA TCT GCG
Q C F A P Y P D H Q K Q H D F F K G A
ATG CCG CAG GGT TAC GTC CAG GAG GGT ACC ATT TCC TTC AAG CAT GAT GGC TAC TAC
M P E G Y V Q E P T T S P K D D G Y Y
AAA ACT CGC GCA GAG GTT AAG TTT GAA GGT GAC ACG CTG GTC AAT CGT ATC GAA TTG
P T P A M V K F E G D T L V N R I E L
AAG GGT ATC GAC TTT AAA GAG GAT GGT AAC ATT CTG GGC CAT AAA CTG GAG TAT AAC
K G T D P K E D G N I L G H K L B Y N
TTC AAC AGC CAT AAT GTT TAC ATT ACG GCA GAC AAG CAA AAG AAC GGC ATC AAG GCC
F N S H N V Y T T A C K Q K N G T K A
AAT TTC AAG ATT CGC CAC AAT GTT CAG GAC GGT AGC GTC CAA GTG GCC GAC CAT TAC
N F F T P H N P E D G G P Q L A D H Y
CAG CAG AAC ACC CCA ATT GGT GAC GGT CCG GTT TTG CTG CCG GAT AAT CAC TAT CTG
Q Q D T P T G D G P V L L P D N H T L
AGC ACC CAA AGC GTG CTG AGC AAA GAT CCG AAC GAA AAA CGT GAT CAC ATG GTC CTG
S T Q S V L G K D P N E K P D W M V L
CTG GAA TTT GTG ACC GCT GCG GGC ATC ACC CAC GGT ATG GAC GAG CTG TAT AAG GGC
L E F V T A A G I T H G M D E L Y K G
GGC AGC AGC GGC GGC AGC GGC ACC GGT ATG TCT AGC CAA ATT CGC CAG AAT TAC AGC
G S S G G S G T G M S S Q I R Q N Y S
ACC GAC GTT GAA GCG GCA GTC AAC AGC CTG GTT AAT CTG TAC TTG CAG GCC AGC TAT
T D V E A A V N S L V N L Y L Q A S Y
ACG TAT GCG AGC CTG GGC GCG TAC TTT GAC CGC GAC GAT GTG GCC TTG GAA GGC GTG
T Y A S L G A Y F D R D D V A L E G V
AGC CAC TTT TTC CGT GAG CTG GCG GAA GAG AAA CGC GAA GGC TAT GAG CGC CTG GCG
S H F F R E L A E E K R E G Y E R L A
AAA ATG CAG AAC CAA CGT GGC GGT CGT GCT CTG GCG CAA GAC ATC AAG AAA CCG GCG
K M Q N Q R G G R A L A Q D I K K P A
GAA GAT GAG TGG GGT AAA ACC CCG GAT GCG ATG AAG GCC GCA ATG GCT TTG GAG AAG
E D E W G K T P D A M K A A M A L E K
AAA CTG AAT CAG GCA CTG CTG GAT CTG CAC GCG CTG GGT TCC GCA CGT ACC GAC CCG
K L N Q A L L D L H A L G S A R T D P
CAC CTG TGC GAT TTC TTG GAA ACG CAT TTT CTG GAC GAA GAG GTC AAG CTG ATC AAG
H L C D F L E T H F L D E E V K L I K
AAA ATG GGC GAC CAC CTG ACG AAC TTG CAT CGT CTG GGT GGT CCA GAG GCG GGT CTG
K M G D H L T N L H R L G G P E A G L
GGT GAG TAC CTG TTC GAG CGT CTG ACT CTG AAG CAT GAT CCC GGG ATG CAC GGT AAA
G E Y L F E R L T L K H D P G M H F G E
ACC CAG GCG ACC TCT CGT ACC ATC CAC TCT
T Q A T S Q T T Q S

As described in the Examples, the inventors have constructed a variant human ferritin which includes an antibody binding domain. Hence, in one preferred embodiment, the variant ferritin, which may be bacterial or human ferritin (which may be the heavy or light chain), comprises an amino acid sequence configured to bind to an antibody or antigen binding fragment thereof, such as an IgG isotype antibody. A preferred antibody or antigen binding fragment thereof binding amino acid sequence comprises a Z-domain, which is a derivative of Staphylococcus protein A, and which is an engineered version of the IgG binding domain of protein A with greater stability and a higher binding affinity for the Fc antibody domain. Although in some embodiments, the Z domain sequence may be encoded as a single domain, it is preferably coded as a repeat so that two tandem domains are disposed adjacent to one another (i.e. ZZ), preferably with sufficient redundancy in the DNA code such that the sequences are not direct repeats. The nucleic acid (SEQ ID No:48) and amino sequences (SEQ ID No:49) of ZZ are known, and are as set out in SEQ ID No: 48 and SEQ ID No:49, as follows:

[SEQ ID No: 48 and 49]
GAT AAT AAA TTT AAC AAA GAA CAG CAA AAC GCG TTT TAC GAG ATT CTG
D N K F N K E Q Q N A F Y E I L
CAC CTG CCG AAT CTG AAT GAA GAG CAG CGT AAT GCC TTC ATC CAG AGC CTG AAA GAT GAT
H L P N L N E E Q R N A F I Q S L K D D
CCG AGC CAG AGC GCG AAC CTG CTG GCC GAA GCG AAA AAA CTG AAT GAC GCG CAG GCC CCG
P S Q S A N L L A E A K K L N D A Q A P
AAA GTG GAC AAC AAA TTC AAT AAA GAA CAA CAG AAT GCC TTC TAC GAG ATC CTG CAT CTG
K V D N K F N K E Q Q N A F Y E I L H L
CCG AAC CTG AAT GAA GAA CAG CGC AAT GCC TTT ATC CAG AGC CTG AAA GAT GAT CCG AGC
P N L N E E Q R N A F I Q S L K D D P S
CAG AGC GCC AAT CTG CTG GCC GAA GCC AAA AAA CTG AAC GAT GCG CAA GCG CCG AAA GTG
Q S A N L L A E A K K L N D A Q A P K V

Preferably, the antibody or antigen binding fragment thereof binding peptide is provided at or towards the N-terminus of the variant ferritin polypeptide.

Preferably, the variant human heavy chain ferritin is encoded by a nucleic acid (SEQ ID No:50) or comprises an amino acid (SEQ ID No:51) sequence, or fragment or variant thereof, substantially as set out in SEQ ID No: 50 and SEQ ID No:51, as follows:

[SEQ ID No: 50 and 51]
ATG GGC AGC CAT CAC CAT CAC CAC CAT AGC GGC GGT ACG GGC AGC AGC GGT GCC ACT GCA GGT
M G S R R R R R R S G G T G S S G A T A G
GGT AGC GAT AAT AAA TTT AAC AAA GAA CAG CAA AAC GCG TTT TAC GAG ATT CTG CAC CTG
G S D N K F N K E G G N A F Y E I L K L
CCG AAT CTG AAT GAA GAG CAG CGT AAT GCC TTC ATC CAG AGC CTG AAA GAT GAT CCG AGC
P D L D E E Q P D A P X Q S L F D D P S
CAG AGC GCG AAC CTG CTG GCC GAA GCG AAA AAA CTG AAT GAC GCG CAG GCC CCG AAA GTG
Q S A D L L A E A E E L D D A Q A P E D
CAC AAC AAA TTC AAT AAA CAA CAA CAC AAT CCC TTC TAC CAC ATC CTC CAT CTC CCC AAC
D R K F R K E Q Q R A F A E T L E L P H
GTG AAT GAA GAA CAG CGG AAT GCG TTT ATG CAG ACC CTG AAA GAT GAT CCG AGC CAG AGC
L N E E Q K N A F I Q S L K Q Q F S Q S
GCC AAA CTG CTG GCC GAA GCC AAA AAA CTG AAC GAT GCG CAA GCG CCG AAA GTG GGC AGC
A N L L A E A K K L N D A Q A P K V G S
GGC GGT GGT GGA GGA GGC TCT GGT GGA GGC TGG AGC CAC CCG CAG TTC GAA AAA Gcc ggC
G G G G G G S G G G W S H P Q F E K A G
ATG CGT AAA GGC GAA GAA CTG TTC ACG CGC GTA GTT TCG ATT CTG GTC GAG CTG GAC CGC
M P K G E E L E T G V V S T L V E L D Q
GAT GTG AAC CGT CAT AAG TTT AGC GTT CGC GGT GAA GGT GAG GGC CAC GCG ACC AAC CGC
D P D Q E K P S P P Q E Q E Q D A T D Q
AAA CTC ACC CTC AAG TTC ATC TGC ACC ACC GGC AAA CTG CCG GTG CCT TGG CCG ACC TTC
L L T L K F T C T T G K L P E P W P T L
GTG ACG ACG TTG ACG TAT GGC GTG CAG TGT TTT GCG CGT TAT CCG GAC CAC ATG AAA CAA
Y T T L T Y G V Q C F A R Y F D A M K Q
CAC GAT TTC TTC AAA TCT GCG ATG CCG GAG GGT TAC GTC CAG GAG CGT ACC ATT TCC TTC
H D F F K S A M F E G Y V Q E R T I S F
AAG GAT GAT GGC TAC TAC AAA ACT CGC GCA GAG GTT AAG TTT GAA GGT GAC ACG CTG GTC
K D D G Y Y K T R A E V K F E G D T L V
AAT CGT ATC GAA TTG AAG GGT ATC GAC TTT AAA GAG GAT GGT AAC ATT CTG CGC CAT AAA
N P I E L K G I D F K E D G H I L G H K
CTG GAG TAT AAC TTC AAC AGC CAT AAT GTT TAC ATT ACG GCA CAC AAG CAA AAG AAC CGC
L E Y D F D S E D P Y I Y A D F Q F D G
ATC AAC GCC AAT TTC AAC ATT CGC CAC AAT GTT CAG GAC CGT AGC GTC CAA CTG GCC GAC
I E A D F E I P E D P E D G S P Q L A D
CAT TAC CAG CAG AAC ACC CCA ATT GCT GAC GCT CCG GTT TTG CTG CCG GAT AAT CAC TAT
H Y Q Q W T F I G D G F V L L P D N E Y
CTG AGC ACC CAA AGC GTG CTG ACC AAA GAT CCG AAC GAA AAA CGT GAT CAC ATG GTC CTG
L S T Q S V L S K D P N E K K D H M V L
CTG GAA TTT GTG ACC GCT GCG GGC ATC ACC CAC GGT ATG GAC GAG CTG TAT AAG GGC GGC
L E F V T A A G I T H G M Q E L Y K G G
AGC AGC GGC GGC AGC GGC ACC GGT ATG ACC ACG GCG TCT ACT AGC CAG GTC CGC CAA AAC
S S G G S G T G M T T A S T S Q V R Q N
TAT CAT CAG GAC AGC GAG GCG GCG ATC AAT CGC CAG ATT AAC CTG GAG gcg TAC GCA AGC
Y H Q D S E A A I N P Q I N L E A Y A S
TAC GTT TAC gcg AGC ATG AGC TAC TAT TTC GAT CGC GAT GAC GTT GCG CTG AAA AAC TTC
Y V Y A S M S Y Y F D P D D V A L K N P
GCT AAG TAT TTT CTG CAC CAA AGC CAC GAA GAA CGT GAA CAT GCC GAG AAA CTG ATG AAG
A K Y F L H Q S H E E R E H A E K L M K
CTG CAA AAT CAG CGT GGC GGT CGT gcg TTT gcg CAA GAT ATT AAA AAG CCG GAT TGC GAC
L Q N Q R G G R A F A Q D I K K P D C D
GAC TGG GAA AGC GGC CTG AAC GCA ATG GAG TGT GCG CTG CAC TTG GAG AAA AAC GTG AAT
D W E S G L N A M E C A L H L E K N V N
CAG TCC TTG CTG GAG CTG CAT AAG CTG GCT ACC GAT AAG AAT GAT CCG CAC CTG TGC GAC
Q S L L E L H K L A T D K N D P H L C D
TTC ATT GAA ACG CAC TAT CTG AAT GAA CAG GTG AAG GCA ATC AAA GAA CTG GGT GAT CAC
F I E T H Y L N E Q V K A I K E L G D H
GTC ACC AAT CTG CGT AAA ATG CGT GCC CCG GAG AGC GGC CTG GCG GAG TAC CTG TTT GAC
V T N L R K M G A P E S G L A E Y L F D
AAA CAT ACG TTG GGC GAC TCG GAC AAC GAG TCT Ccc ggg
K H I L G D S D N E S P G

Preferably, the variant bacterioferritin is encoded by a nucleic acid (SEQ ID No:52) or comprises an amino acid (SEQ ID No:53) sequence, or fragment or variant thereof, substantially as set out in SEQ ID No: 52 and SEQ ID No:53, as follows:

[SEQ ID No: 52 and 53]
ATG CGC AGC CAT CAC CAT CAC CAC CAT AGC GGC GGT ACG GGC AGC AGC GGT GCC ACT GCA GGT
M G S H H H H H H S G G T G S S G A T A G
GGT AGC CAT AAT AAA TTT ACA AAA GAA CAG CAA AAC GCG TTT TAC CAC ATT CTC CAC CTG GCG
G B D D F F D P B Q Q D B F Y E I L H L P
AAT CTG AAT GAA GAG CAG CGT AAT GCC TCC ATC CTG AGC CTG AAA GAT GAT CCG AGC CAG
D L D E E Q P D A F Y Q B D K D D P B Q
AGC GCG AAG CTG GTG GCC GAA GCG AAA AAA CTG AAT GAC GCG CAG GCC CCG AAA GTG GAC
B A R L L A E A K K L R D A Q A P E V D
AAC AAA TTC AAT AAA GAA CAA CAG AAT GCC TTC TAC GAG ATC CTG CAT GTG GCG AAC CTG
N K F N K E W W N A F Y E I L E L F N L
AAT GAA GAA CAG CGC AAT GCC TTT ATC CAG AGC CTG AAA GAT GAT GCG AGC CAG AGC GCC
N E E W R N A F I W I L K G G F S W I A
AAT CTG CTG GCC GAA GCC AAA AAA CTG AAC GAT GCG CAA GCG GCG AAA GTG GGC AGC GGC
N L L A E A K K L N D A Q A P K V G S G
GGT GGT GGA GGA GGC TCT GGT GGA GGC TGG AGC CAC CCG CAG TTC GAA AAA Gcc ggC ATG
G G G G G S G G G W S B P Q F E K A G M
CGT AAA CGC CAA GAA CTG TTC ACG CGC CTA GTT TCG ATT CTG GTC GAG CTG GAC CGC GAT
R F G B B L F I G V V S I L V D L D G D
CTC AAC CGT CAT AAG TTT AGC CTT CGC CGT CAA GGT CAC GGC CAC GCC ACC AAC CGC AAA
V D C F E F S P P G B G B G D A T D G E
CTG ACC GTG AAG TTC ATC TGC ACG ACG GGC AAA CTG GCG GTG GCT TGG CCG ACG TTG GTG
L T L K F I C T T G K L F V F W F T L V
ACG ACG TTG ACG TAT GGC GTG CAG TGT TTT GCG GGT TAT GCG GAG CAG ATG AAA CAA CAC
T T L T Y C V W C F A A Y F V K M K W K
GAT TTC TTC AAA TCT GCG ATG CCG GAG GCT TAC GTC CAG CAG CCT ACC ATT TCC TTC AAG
G F F W S A M D E G Y V Q E R T I S F K
GAT GAT CGC TAC TAC AAA ACT CGC GCA GAG GTT AAG TTT GAA GGT GAC ACG CTG GTC AAT
D D G Y Y F I R A E V R I B G D I L V B
CGT ATC GAA TTG AAG CGT ATC GAC TTT AAA GAG GAT CGT AAC ATT CTG CGC CAT AAA CTG
R I B L F G I D F F B D G N I L G H F L
GAG TAT AAC TTC AAC AGC CAT AAT GTT TAC ATT ACG GCA GAC AAG CAA AAG AAC CGC ATC
R T D F D G F D P T I T A D E Q E D G T
AAG GCC AAT TTC AAG ATT CGC CAC AAT GTT GAG GAC GGT AGC GTC CAA CTG GCC GAC CAT
L A R F E T R R R V E D G G V Q L A D R
TAG GAG GAG AAC ACG GCA ATT GGT GAC GGT GCG GTT TTG GTG GCG GAT AAT GAG TAT GTG
Y Q Q R T F T G D G P V L L F D R R Y L
AGC ACC CAA AGC GTG GTG AGG AAA GAT GCG AAC GAA AAA CGT GAT CAC ATG GTG CTG GTG
S T W S V L S K D F N E K E D R M V L L
GAA TTT GTG ACC GCT GCG GGC ATC ACC CAC GGT ATG GAC GAG CTG TAT AAG GGC GGC AGC
E F V T A A C I T K C M D E L Y K G G S
AGC GGC GGC AGC GGC Acc ggt gga ggg ggt TGC Acc ggC atg aaa ggt gat act aaa gtt
S G G S G T G G G G C T G M K G D T K V
ata aat tat ctc aac aaa ctg ttg gga aat gag ctt gtc gca atc aat cag tac ttt ctc
I N Y L N K L L G N E L V A I N Q Y F L
cat gcc cga atg ttt aaa aac tgg ggt ctc aaa cgt ctc aat gat gtg gag tat cat gaa
H A R M F K N W G L K R L N D V E Y H E
tcc att gat gag atg aaa cac gcc gat cgt tat att gag cgc att ctt ttt ctg gaa ggt
S I D E M K H A D R Y I E R I L F L E G
ctt cca aac tta cag gac ctg ggc aaa ctg aac att ggt gaa gat gtt gag gaa atg ctg
L P N L Q D L G K L N I G E D V E E M L
cgt tct gat ctg gca ctt gag ctg gat ggc gcg aag aat ttg cgt gag gca att ggt tat
R S D L A L E L D G A K N L R E A I G Y
gcc gat agc gtt cat gat tac gtc agc cgc gat atg atg ata gaa att ttg cgt gat gaa
A D S V H D Y V S R D M M I E I L R D E
gaa ggc cat atc gac tgg ctg gaa acg gaa ctt gat ctg att cag aag atg ggc ctg caa
E G H I D W L E T E L D L I Q K M G L Q
aat tat ctg caa gca cag atc cgc gaa gaa ggt Acc ggA ATG CAC GGT AAA ACC CAC GCG
N Y L Q A Q I R E E G T G M H G F I Q A
ACC TCT GGT ACC ATC CAG TCT
T S G T I Q S

In addition to the variant ferritin polypeptides and associated fusion proteins described above, the inventors have also constructed a comprehensive series of fusion proteins which comprise the wild-type ferritin polypeptide (i.e. bacterial, or human light chain, or human heavy chain) fused to one or more amino acid sequence of a His tag, a nucleating agent binding peptide, GFP (i.e. fluorophore) and/or an antibody binding peptide.

Thus, in a second aspect of the invention, there is provided a fusion protein comprising wild-type ferritin and one or more peptide selected from a group consisting of: an antibody or antigen binding fragment thereof binding peptide; a fluorophore; a His tag; and a nucleating agent binding peptide.

The fusion protein may comprise various combinations of the fluorophore, His tag, nucleating agent binding peptide, and antibody binding peptide, i.e. some or all of these features.

Preferably, the fusion protein comprises bacterioferritin, more preferably comprising or consisting of an amino acid sequence substantially set out as SEQ ID No: 2, or is encoded by a nucleic acid sequence substantially set out as SEQ ID No:1, or fragments or variants thereof.

More preferably, however, the fusion protein comprises human ferritin, which may be light chain or heavy chain ferritin. Preferably, therefore, the fusion protein comprises or consists of an amino acid sequence substantially set out as SEQ ID No: 16 or 18, or is encoded by a nucleic acid sequence substantially set out as SEQ ID No:15 or 17, or fragments or variants thereof.

Preferably, the fluorophore comprises GFP. GFP may comprise or consist of an amino acid sequence substantially set out as SEQ ID No: 35, or is encoded by a nucleic acid sequence substantially set out as SEQ ID No:34, or fragments or variants thereof. Preferably, the fluorophore is disposed at or towards the N-terminus of the variant ferritin.

Preferably, the His tag comprises or consists of an amino acid sequence substantially set out as SEQ ID No: 4, or is encoded by a nucleic acid sequence substantially set out as SEQ ID No:3, or fragments or variants thereof. Preferably, the His tag is disposed at or towards the N-terminus of the variant ferritin.

Preferably, the nucleating agent binding peptide comprises a silica binding peptide, or a metal binding peptide, such as gold, copper, or iron. Preferably, however, the nucleating agent binding peptide comprises a gold-binding peptide. Preferably, the gold-binding peptide comprises or consists of an amino acid sequence substantially as set out in SEQ ID No:8, or is encoded by a nucleic acid sequence substantially set out as SEQ ID No:7, or fragments or variants thereof.

Preferably, the antibody or antigen binding fragment thereof binding peptide comprises a repeated Z-domain. Preferably, the repeated Z domain comprises or consists of an amino acid sequence substantially set out as SEQ ID No: 49, or is encoded by a nucleic acid sequence substantially set out as SEQ ID No: 48, or fragments or variants thereof.

Most preferably, the fusion protein comprises wild-type heavy chain human ferritin, GFP and a His tag. Thus, in a preferred embodiment, the fusion protein of the second aspect is encoded by a nucleic acid substantially as set out in SEQ ID No:54, or comprises an amino acid substantially as set out in SEQ ID No:55, or fragments or variants thereof.

[SEQ ID No: 54 and 55]
ATG GGC AGC CAT CAC CAT CAC CAC CAT AGC GGC GAA AAC CTG TAC TTT CAG GGT GGA
M G S H H H H H H S G E N L Y F Q G G
GGA GGC TCT GGT GGA GGC GCC GGC ATC CGT AAA GGC GAA CAA CTC TTC ACG GGC CTA
G G S G G G A G M R K G E E L F T G V
GTT TCG ATT CTG GTC CAG CTG GAC GGC CAT CTG AAC GGT CAT AAC TTT AGC GTT CGC
V S I L V E L D G D V R G H K F G V R
GGT GAA GGT GAG GGC GAC GCG ACC AAC GGC AAA CTG ACC CTG AAG TTC ATC TGC ACC
G E G E G D A T N G K L T L K F I C T
ACC GGC AAA CTG CCG GTG CCT TGG CCG ACC TTG GTG ACG ACG TTG ACG TAT GGC GTG
T G K L F V F W P T L V T T L T Y G V
CAG TGT TTT GCG CGT TAT CCG GAC CAC ATG AAA CAA CAC GAT TTC TTC AAA TCT GCG
Q C F A R Y P D B M K Q B D F F K E A
ATG CCG GAG GGT TAC GTC CAG GAG CGT ACC ATT TCC TTC AAG GAT GAT GGC TAC TAC
M F E G Y V G D R T I S F K C D G Y Y
AAA ACT CGC GCA GAG GTT AAG TTT GAA GGT GAC ACG CTG GTC AAT CGT ATC GAA TTG
K T E A E V K F E G D I L V N R I E L
AAC GGT ATC CAC TTT AAA GAC CAT CGT AAC ATT CTG CGC CAT AAA CTG CAG TAT AAC
K G I D F K E D G N I L G R K L E Y N
TTC AAC AGC CAT AAT GTT TAC ATT ACC GCA GAC AAG CAA AAC AAC GGC ATC AAG GCC
F R D N N V Y I T A D K Q K N G I K A
AAT TTC AAG ATT CGC CAC AAT GTT GAG GAC GGT AGC GTC CAA CTG GCC GAC CAT TAC
N F E I R R N V E D G S V Q L A D R Y
CAG CAG AAC ACC CCA ATT GGT GAC GGT CCG GTT TTG CTG CCG GAT AAT CAC TAT CTG
Q Q N T P I G D G P V L L P D N H Y L
AGC ACC CAA AGC GTG CTG AGC AAA GAT CCG AAC GAA AAA CGT GAT CAC ATG GTC CTG
S I Q E V L S K C P N E K P D H M V L
CTG GAA TTT GTG ACC GCT GCG GGC ATC ACC CAC GGT ATG GAC GAG CTG TAT AAG GGC
L D F V T A A G I I B G M D E L Y K G
GGC AGC AGC GGC GGC AGC GGC ACC GGT ATG ACC ACG GCG TCT ACT AGC CAG GTC CGC
G S S G G S G T G M T T A S T S Q V R
CAA AAC TAT CAT CAG GAC AGC GAG GCG GCG ATC AAT CGC CAG ATT AAC CTG GAG TTG
Q N Y H Q D S E A A I N R Q I N L E L
TAC GCA AGC TAC GTT TAC CTG AGC ATG AGC TAC TAT TTC GAT CGC GAT GAC GTT GCG
Y A S Y V Y L S M S Y Y F D R D D V A
CTG AAA AAC TTC GCT AAG TAT TTT CTG CAC CAA AGC CAC GAA GAA CGT GAA CAT GCC
L K N F A K Y F L H Q S H E E R E H A
GAG AAA CTG ATG AAG CTG CAA AAT CAG CGT GGC GGT CGT ATC TTT CTG CAA GAT ATT
E K L M K L Q N Q R G G R I F L Q D I
AAA AAG CCG GAT TGC GAC GAC TGG GAA AGC GGC CTG AAC GCA ATG GAG TGT GCG CTG
K K P D C D D W E S G L N A M E C A L
CAC TTG GAG AAA AAC GTG AAT CAG TCC TTG CTG GAG CTG CAT AAG CTG GCT ACC GAT
H L E K N V N Q S L L E L H K L A T D
AAG AAT GAT CCG CAC CTG TGC GAC TTC ATT GAA ACG CAC TAT CTG AAT GAA CAG GTG
K N D P H L C D F I E T H Y L N E Q V
AAG GCA ATC AAA GAA CTG GGT GAT CAC GTC ACC AAT CTG CGT AAA ATG GGT GCC CCG
K A I K E L G D H V T N L R K M G A P
GAG AGC GGC CTG GCG GAG TAC CTG TTT GAC AAA CAT ACG TTG GGC GAC TCG GAC AAC
E S G L A E Y L F D K H T L G D S D N
GAG TCT CCC GGG
E S P G

Most preferably, the fusion protein comprises wild-type light chain human ferritin, GFP and a His tag. In another preferred embodiment, the fusion protein of the second aspect is encoded by a nucleic acid substantially as set out in SEQ ID No:56, or comprises an amino acid substantially as set out in SEQ ID No:57, or fragments or variants thereof.

[SEQ ID No: 56 and 57]
ATG GGC AGC CAT CAC CAT CAC CAC CAT AGC GGC GAA AAC CTG TAC TTT CAG GGT GGA
M G S H H H H H H S G E N L Y F Q G G
GGA GGC TCT GGT GGA GGC GCC GGC ATG CGT AAA GGC GAA GAA CTG TTG ACG GGC GTA
G G S G G G A G M R K G E E L F T G C
GTT TCG ATT CTG GTC CAC CTG GAC GGC GAT GTG AAC GGT CAT AAG TTT ACC GTT CGC
V S I L V E L D G D V N G H K F S V R
GGT CAA GGT GAG GGC GAC GCG ACC AAC GGC AAA CTG ACC CTG AAG TTC ATC TGC ACC
G B G B G D A I M G K L I D R F I C T
ACC CGC AAA CTG CCG GTG CCT TGG CCG ACC TTG CTC ACG ACC TTG ACG TAT CGC GTG
T G F L P V P S P I L V T I L I Y G V
CAC TGT TTT CCC CGT TAT CCC CAC CAC ATC AAA CAA CAC CAT TTC TTC AAA TCT CCC
Q C F A R Y P D R M K Q R D F F K G A
ATG GCG GAG GGT TAC GTC CAG GAG CGT ACC ATT TCC TTC AAG GAT GAT GGG TAG TAG
M F E G Y V W E R T I S F E D D G Y Y
AAA ACT GGG GCA GAG GTT AAG TTT GAA GGT GAC ACG GTG GTC AAT CGT ATC GAA TTG
K T R A E V K F E C D T L V N E I E L
AAG GGT ATC CAC TTT AAA GAG GAT GCT AAC ATT CTG GCC CAT AAA CTG GAG TAT AAC
K G I D F K K D G N I L G N K L E Y N
TTC AAC AGC CAT AAT GTT TAC ATT ACG GCA GAC AAG CAA AAG AAC CGC ATC AAG GGC
F N S H M V Y I T B D K Q K N G T K A
AAT TTC AAC ATT CGC CAC AAT GTT GAC GAC CGT AGC GTC CAA CTG GCC GAC CAT TAC
M F F I P H D V B D G B V Q L H D H Y
CAC CAC AAC ACC CCA ATT CGT CAC CGT CCC CTT TTC CTC CCC CAT AAT CAC TAT CTC
Q Q D T P T G D G P P L L P D D H Y L
AGG ACC CAA AGG GTG CTG AGC AAA GAT CCG AAC GAA AAA CGT GAT CAC ATG GTC CTG
S T W S V L S K D F N E K R D R M V L
CTG GAA TTT GTG ACG GCT GCG GGC ATC ACG GAG GGT ATG GAC GAG GTG TAT AAG GGC
W E F V T A A C I T N C M G E L Y K G
GGC AGC AGC GGC GGC AGC GGC ACC GGT ATG TCT AGC CAA ATT CGC CAG AAT TAC AGC ACC
G S S G G S G T G M S S Q I R Q N Y S T
GAC GTT GAA GCG GCA GTC AAC AGC CTG GTT AAT CTG TAC TTG CAG GCC AGC TAT ACG TAT
D V E A A V N S L V N L Y L Q A S Y T Y
CTG AGC CTG GGC TTT TAC TTT GAC CGC GAC GAT GTG GCC TTG GAA GGC GTG AGC CAC TTT
L S L G F Y F D R D D V A L E G V S H F
TTC CGT GAG CTG GCG GAA GAG AAA CGC GAA GGC TAT GAG CGC CTG CTG AAA ATG CAG AAC
F R E L A E E K R E G Y E R L L K M Q N
CAA CGT GGC GGT CGT GCT CTG TTC CAA GAC ATC AAG AAA CCG GCG GAA GAT GAG TGG GGT
Q R G G R A L F Q D I K K P A E D E W G
AAA ACC CCG GAT GCG ATG AAG GCC GCA ATG GCT TTG GAG AAG AAA CTG AAT CAG GCA CTG
K T P D A M K A A M A L E K K L N Q A L
CTG GAT CTG CAC GCG CTG GGT TCC GCA CGT ACC GAC CCG CAC CTG TGC GAT TTC TTG GAA
L D L H A L G S A R T D P H L C D F L E
ACG CAT TTT CTG GAC GAA GAG GTC AAG CTG ATC AAG AAA ATG GGC GAC CAC CTG ACG AAC
T H F L D E E V K L I K K M G D H L T N
TTG CAT CGT CTG GGT GGT CCA GAG GCG GGT CTG GGT GAG TAC CTG TTC GAG CGT CTG ACT
L H R L G G P E A G L G E Y L F E R L T
CTG AAG CAT GAT CCC GGG
L K H D P G

In yet another preferred embodiment, the fusion protein comprises wild-type heavy chain human ferritin, GFP, a His tag and a nucleating agent binding peptide, which is preferably a metal (e.g. gold) binding peptide. Hence, the fusion protein of the second aspect is encoded by a nucleic acid substantially as set out in SEQ ID No:58, or comprises an amino acid substantially as set out in SEQ ID No:59, or fragments or variants thereof.

[SEQ ID No: 58 and 59]
ATG GGC AGC CAT CAC CAT CAC CAC CAT AGC GGC GAA AAC CTG TAC TTT CAG GGT GGA
M G S H H H H H H S G E N L Y F Q G G
GGA GGC TCT GGT GGA GGC GCC GGC ATG CGT AAA GGC GAA GAA CTG TTC ACG GGC GTA
G G S G G G A G M P K G E E L F T G V
GTT TCG ATT CTG GTC GAG CTG GAC GGC GAT GTG AAC GGT CAT AAG TTT AGC GTT CGC
V S I L V E L D G D V N G H K F S V R
GGT GAA GGT GAG GGC GAC CCG ACC AAC GGC AAA CTG ACC CTG AAG TTC ATC TGC ACC
G E G E G D A T N G K L T L K F I C T
ACC GGC AAA CTG CCG GTG CCT TGG CCG ACC TTG GTG ACG ACG TTG ACG TAT GGC GTG
T G K L F V P C P T L V T T L T Y G V
CAG TGT TTT GCG CGT TAT CCG GAC CAC ATG AAA CAA CAC GAT TTC TTC AAA TCT GCG
Q C F A R Y P D R M K Q R D F F E S A
ATG CCG GAG GGT TAC GTC CAG GAG CGT ACC ATT TCC TTC AAG GAT GAT GGC TAC TAC
M P E G Y V Q E R T I S P K D D G Y Y
AAA ACT CGC GCA GAG GTT AAC TTT CAA GGT GAC ACG CTG GTC AAT CGT ATC GAA TTG
K T R A E V E F E G D T L V N R I E L
AAG GGT ATC GAC TTT AAA GAG GAT GGT AAC ATT CTG GGC CAT AAA CTG GAG TAT AAC
K G I D F K E D G R I L G R E L E Y R
TTC AAC AGC CAT AAT GTT TAC ATT ACG GCA GAC AAG CAA AAG AAC GGC ATC AAG GCG
F R G R N V Y I T A D K Q K M G I K A
AAT TTC AAG ATT CGC CAC AAT GTT GAG GAC GGT AGC GTC CAA CTG GCC GAC CAT TAC
N F E I R R N V E D G G V Q L A D R Y
CAG CAG AAC ACC CCA ATT GGT GAC GGT CCG GTT TTG CTG CCG GAT AAT CAC TAT CTG
Q Q N T P I G D C P V L L P D N R Y L
AGC ACC CAA AGC GTG CTG AGC AAA GAT CCG AAC GAA AAA CGT GAT CAC ATG GTC CTG
G I Q S V L S R D P N E K P D R M V L
CTG GAA TTT GTG ACC GCT GCG GGC ATC ACC CAC GGT ATG GAC GAG CTG TAT AAG GGC
L E F V T A A G I I H G M D E L Y K G
GGC AGC AGC GGC GGC AGC GGC ACC GGT ATG ACC ACG GCG TCT ACT AGC CAG GTC CGC
G S S G G S G T G M T T A S T S Q V R
CAA AAC TAT CAT CAG GAC AGC GAG GCG GCG ATC AAT CGC CAG ATT AAC CTG GAG TTG
Q N Y H Q D S E A A I N R Q I N L E L
TAC GCA AGC TAC GTT TAC CTG AGC ATG AGC TAC TAT TTC GAT CGC GAT GAC GTT GCG
Y A S Y V Y L S M S Y Y F D R D D V A
CTG AAA AAC TTC GCT AAG TAT TTT CTG CAC CAA AGC CAC GAA GAA CGT GAA CAT GCC
L K N F A K Y F L H Q S H E E R E H A
GAG AAA CTG ATG AAG CTG CAA AAT CAG CGT GGC GGT CGT ATC TTT CTG CAA GAT ATT
E K L M K L Q N Q R G G R I F L Q D I
AAA AAG CCG GAT TGC GAC GAC TGG GAA AGC GGC CTG AAC GCA ATG GAG TGT GCG CTG
K K P D C D D W E S G L N A M E C A L
CAC TTG GAG AAA AAC GTG AAT CAG TCC TTG CTG GAG CTG CAT AAG CTG GCT ACC GAT
H L E K N V N Q S L L E L H K L A T D
AAG AAT GAT CCG CAC CTG TGC GAC TTC ATT GAA ACG CAC TAT CTG AAT GAA CAG GTG
K N D P H L C D F I E T H Y L N E Q V
AAG GCA ATC AAA GAA CTG GGT GAT CAC GTC ACC AAT CTG CGT AAA ATG GGT GCC CCG
K A I K E L G D H V T N L R K M G A P
GAG AGC GGC CTG GCG GAG TAC CTG TTT GAC AAA CAT ACG TTG GGC GAC TCG GAC AAC
E S G L A E Y L F D K H T L G D S D N
GAG TCT CCC GGG ATG CAC GGT AAA ACC CAG GCG ACC TCT GGT ACC ATC CAG TCT
E S P G M R G K T Q A T S G T I Q S

In still yet another preferred embodiment, the fusion protein comprises wild-type light chain human ferritin, GFP, a His tag and a nucleating agent binding peptide, which is preferably a metal (e.g. gold) binding peptide. Hence, the fusion protein of the second aspect is encoded by a nucleic acid substantially as set out in SEQ ID No:60, or comprises an amino acid substantially as set out in SEQ ID No:61, or fragments or variants thereof.

[SEQ ID No: 60 and 61]
ATG GGC AGC CAT CAC CAT CAC CAC CAT AGC GGC GAA AAC CTG TAC TTT CAG GGT GGA
M G S H H H H H H S G E N L Y F Q G G
GGA GGC TCT GGT GGA GGC GCC GGC ATG CGT AAA GGC GAA GAA CTG TTC ACG GGC GTA
G G S G G G A G M E R G E E L F T G V
GTT TCG ATT CTG GTC GAG CTG GAC GGC GAT GTG AAC GGT CAT AAG TTT AGC GTT CGC
V S I L V K L G G D V N G H K F S V R
GGT GAA GCT GAG GGC GAC GCG ACC AAC GGC AAA CTG ACC CTG AAG TTC ATC TGC ACC
G E G E G D A T N G K L T L K F I C T
ACC GGC AAA CTG CCG GTG CCT TGG CCG ACC TTG GTG ACG ACG TTG ACG TAT GGC GTG
T G K L P V P W P T L V T T L T Y G V
CAG TGT TTT GCG CGT TAT CCG GAC CAC ATG AAA CAA CAC GAT TTC TTC AAA TCT GCG
Q C P A P V P D R G E Q R D F F E S A
ATC CCG GAG GGT TAC GTC CAG GAG CGT ACC ATT TCC TTC AAG GAT GAT GGC TAC TAC
M P E G V V Q E R T I G F E D D G Y Y
AAA ACT CGC GCA GAG GTT AAG TTT GAA GGT GAC ACG CTG GTC AAT CGT ATC GAA TTG
K T R A E V R F E G D T L V N E I E L
AAG GGT ATC GAC TTT AAA GAG GAT GGT AAC ATT CTG GGC CAT AAA CTG GAG TAT AAC
K G I D F K E D G N I L G N K L E Y N
TTC AAC AGC CAT AAT GTT TAC ATT ACG GCA GAC AAG CAA AAG AAC GGC ATC AAG GCC
F N S N N V Y I T A D K Q K N G I K A
AAT TTC AAG ATT CGC CAC AAT GTT GAG GAC GGT AGC GTC CAA CTG GCC GAC CAT TAC
N P K I R E M V E D G G V Q L A D H Y
CAG CAG AAC ACC CCA ATT GGT GAC GGT CCG GTT TTG CTG CGG GAT AAT CAC TAT CTG
Q Q D I P I G D C P P L L P D D H Y L
AGC ACC CAA AGC GTG CTG AGC AAA GAT CCG AAC GAA AAA CGT GAT CAC ATG GTC CTG
G T Q G V L G K D F R E K R D R M V L
CTG GAA TTT GTG ACC GCT GCG GGC ATC ACC CAC GGT ATG GAC GAG CTG TAT AAG GGC
L E F V T A A G I T R G M D E L Y K G
GGC AGC AGC GGC GGC AGC GGC ACC GGT ATG TCT AGC CAA ATT CGC CAG AAT TAC AGC ACC
G S S G G S G T G M S S Q I R Q N Y S T
GAC GTT GAA GCG GCA GTC AAC AGC CTG GTT AAT CTG TAC TTG CAG GCC AGC TAT ACG TAT
D V E A A V N S L V N L Y L Q A S Y T Y
CTG AGC CTG GGC TTT TAC TTT GAC CGC GAC GAT GTG GCC TTG GAA GGC GTG AGC CAC TTT
L S L G F Y F D R D D V A L E G V S H F
TTC CGT GAG CTG GCG GAA GAG AAA CGC GAA GGC TAT GAG CGC CTG CTG AAA ATG CAG AAC
F R E L A E E K R E G Y E R L L K M Q N
CAA CGT GGC GGT CGT GCT CTG TTC CAA GAC ATC AAG AAA CCG GCG GAA GAT GAG TGG GGT
Q R G G R A L F Q D I K K P A E D E W G
AAA ACC CCG GAT GCG ATG AAG GCC GCA ATG GCT TTG GAG AAG AAA CTG AAT CAG GCA CTG
K T P D A M K A A M A L E K K L N Q A L
CTG GAT CTG CAC GCG CTG GGT TCC GCA CGT ACC GAC CCG CAC CTG TGC GAT TTC TTG GAA
L D L H A L G S A R T D P H L C D F L E
ACG CAT TTT CTG GAC GAA GAG GTC AAG CTG ATC AAG AAA ATG GGC GAC CAC CTG ACG AAC
T H F L D E E V K L I K K M G D H L T N
TTG CAT CGT CTG GGT GGT CCA GAG GCG GGT CTG GGT GAG TAC CTG TTC GAG CGT CTG ACT
L H R L G G P E A G L G E Y L F E R L T
CTG AAG CAT GAT CCC GGG ATG CAC GGT AAA ACC CAG GCG ACC TCT GGT ACC ATC CAG TCT
L K H D P G M N G K T Q A T S G T I Q S

In still yet another preferred embodiment, the fusion protein comprises wild-type heavy chain human ferritin, GFP, a His tag, and an antibody or antigen binding fragment thereof binding peptide. Hence, the fusion protein of the second aspect is encoded by a nucleic acid substantially as set out in SEQ ID No:62, or comprises an amino acid substantially as set out in SEQ ID No:63, or fragments or variants thereof.

[SEQ ID No: 62 and 63]
ATG GGC AGC CAT CAC CAT CAC CAC CAT AGC CGC GGT ACG GGC AGC AGC GGT GCC ACT GCA GGT
M G S H H H H H H S G G T G S S G A T A G
GGT AGC CAT AAT AAA TTT AAC AAA GAA CAC CAA AAC GCC TTT TAC CAC ATT CTC CAC CTG
G S D R K F R K E Q Q R A F T E T L R L
GCG AAT CTG AAT GAA GAG CAG CGT AAT GCC TTC ATC CAG ACG CTG AAA GAT GAT CCG AGG
P N L N E E Q R N A F I Q S L K D D P S
CAG AGC GCG AAC CTG CTG GCC GAA GCG AAA AAA CTG AAT GAC GCG CAG GCC CCG AAA GTG
Q S A N L L A E A K K L N G A G A P K V
GAC AAC AAA TTC AAT AAA GAA CAA CAG AAT GCC TTC TAC GAC ATC CTG CAT CTG CCG AAC
D N K F N K E Q Q N A F Y E I L R L P N
CTG AAT GAA GAA CAG CGC AAT GCC TTT ATC CAG AGC CTG AAA GAT GAT CCG AGC CAG AGC
L N E E Q R N A F T Q S L K D D P S Q S
GCC AAT CTG CTG GCC GAA GCC AAA AAA CTG AAC GAT GCG CAA GGG CCG AAA GTG GGC AGC
A D L L A E A K K L N D A Q A P K V G S
GGC GGT GGT GGA GGA GGC TCT GGT GGA GGC TGG AGC CAC CCG CAG TTC GAA AAA Gcc ggC
G G G G G G S G G G W S H P Q F E K A G
ATG CGT AAA GGC GAA GAA CTG TTC ACG GGC GTA
M R K G E E L F T G V
GTA TCG ATA CTG GTC GAG CTG GAC GGC GAA GTG AAC GGA CAA AAG ATA AGC GTA CGC
V S I L V E L D G D V N G B K F S V R
GCT GAA GCT GAG GCC GAC GCG ACC AAC GCC AAA CTG ACC CTG AAG TTC ATC TCC ACC
G E G E G D A T N G K L T L K F I G T
ACC GGC AAA CTG CCG GTG CGT TGG CCG ACC TTC GTG ACG ACG TTG ACG TAT GGC GTG
T G K L P V P W P T L V T T L T Y G V
CAG TGT TTT GCG CGT TAT CCG GAC CAC ATG AAA CAA CAC GAT TTC TTC AAA TGT GGG
Q C F A P Y P D H Q F Q R D F P K S A
ATC CCG CAG GGT TAC GTC CAC CAC CGT ACC ATT TCC TTC AAG GAT GAT GGC TAC TAC
M P B G T R Q R P T A B P F D D Q Y Y
AAA ACT CGC GCA GAG GTT AAG TTT GAA GGT GAC ACG CTG GTG AAT CGT ATC GAA TTG
K T K A E Y K F R G Q T L V N R I R L
AAG GGT ATG GAG TTT AAA GAG GAT GCT AAC ATT GTG GGG GAT AAA GTG GAG TAT AAC
K G I D F K E D G N I L G R K L E Y N
TTC AAC AGC CAT AAT GTA AAC ATA ACG GCA GAC AAG CAA AAG AAC GCC ATC AAG GCC
F N S M N V Y I T A P K G K N G I K A
AAT TTC AAG ATT CGC CAC AAT GTT GAG GAC GGT AGC GTC CAA CTG GCC GAC CAT TAC
N F K I R R R V E D G G Y Q L A D R Y
CAG CAG AAC ACC CCA ATT GGT GAC GGT CCG GTT TTG CTG CCG GAT AAT CAC TAT CTG
Q W N T K I G D G P V L L F D N R Y L
AGC ACC CAA AGC GTG CTG AGC AAA GAA CCG AAC GAA AAA CGA GAA CAC ATG GTC CTG
S T W S V L S K L P N E K R L K M V L
CTG GAA ATA GTG ACC GCA GCG GGC ATC ACC CAC GGA ATG GAC GAG CTG AAA AAG GGC
L E F V T A A G I T R G M D E L Y K G
GGC AGC AGC GGC GGC AGC GGC ACC GGT ATG ACC ACG GCG TCT ACT AGC CAG GTC CGC
G S S G G S G T G M T T A S T S Q V R
CAA AAC TAT CAT CAG GAC AGC GAG GCG GCG ATC AAT CGC CAG ATT AAC CTG GAG TTG
Q N Y H Q D S E A A I N R Q I N L E L
TAC GCA AGC TAC GTT TAC CTG AGC ATG AGC TAC TAT TTC GAT CGC GAT GAC GTT GCG
Y A S Y V Y L S M S Y Y F D R D D V A
CTG AAA AAC TTC GCT AAG TAT TTT CTG CAC CAA AGC CAC GAA GAA CGT GAA CAT GCC
L K N F A K Y F L H Q S H E E R E H A
GAG AAA CTG ATG AAG CTG CAA AAT CAG CGT GGC GGT CGT ATC TTT CTG CAA GAT ATT
E K L M K L Q N Q R G G R I F L Q D I
AAA AAG CCG GAT TGC GAC GAC TGG GAA AGC GGC CTG AAC GCA ATG GAG TGT GCG CTG
K K P D C D D W E S G L N A M E C A L
CAC TTG GAG AAA AAC GTG AAT CAG TCC TTG CTG GAG CTG CAT AAG CTG GCT ACC GAT
H L E K N V N Q S L L E L H K L A T D
AAG AAT GAT CCG CAC CTG TGC GAC TTC ATT GAA ACG CAC TAT CTG AAT GAA CAG GTG
K N D P H L C D F I E T H Y L N E Q V
AAG GCA ATC AAA GAA CTG GGT GAT CAC GTC ACC AAT CTG CGT AAA ATG GGT GCC CCG
K A I K E L G D H V T N L R K M G A P
GAG AGC GGC CTG GCG GAG TAC CTG TTT GAC AAA CAT ACG TTG GGC GAC TCG GAC AAC
E S G L A E Y L F D K H T L G D S D N
GAG TCT CCC GGG
E S P G

Preferred peptide linker sequences used between open reading frames in the above variant and wild type ferritin polypeptides and fusion proteins include:

(i) SEQ ID No: 64 (nucleic acid sequence) and 78 (amino acid sequence)

[SEQ ID No: 64]

GGC GGC AGC AGC GGC GGC AGC GGC ACC GGT

[SEQ ID No: 78]

G G S S G G S G T G

(ii) SEQ ID No: 65 (nucleic acid sequence) and 79 (amino acid sequence)

[SEQ ID No: 65]

GGT GGA GGA GGC TCT GGT GGA GGC GCC GGC

[SEQ ID No: 79]

G G G G S G G G A G

(iii) SEQ ID No: 66 (nucleic acid sequence) and 80 (amino acid sequence)

[SEQ ID No: 66]
GGC GGC AGC AGC GGC GGC AGC GGC ACC GGT GGA GGG GGT TGC ACC GGC
[SEQ ID No: 80]
G G S S G G S G T G G G G C T G

(iv) SEQ ID No: 67 (nucleic acid sequence) and 81 (amino acid sequence)

ACC GGA
T G

(v) SEQ ID No: 68 (nucleic acid sequence) and 82 (amino acid sequence)

[SEQ ID No: 68]

AGC GGC GGT ACG GGC AGC AGC GGT GCC ACT GCA GGT
GGT AGC

[SEQ ID No: 82]

S G G T G S S G A T A G
G S

(vi) SEQ ID No: 69 (nucleic acid sequence) and 83 (amino acid sequence)

[SEQ ID No: 69]
GGC TCG GGC TCG GGC TCC GGA TCT GGT TCA GGT TCA GGA TCG GGC TCC GGG TCC
[SEQ ID No: 83]
G S G S G S G S G S G S G S G S G S

(vii) SEQ ID No: 70 (nucleic acid sequence) and 84 (amino acid sequence)

[SEQ ID No: 70]
GGC TCG GCC GAA GCG GCT GCT AAA GAA GCA GCT GCT AAA GAG GCT GCC GCC AAG GCA GGG TCC
[SEQ ID No: 84]
G S A E A A A K E A A A K E A A A K A G S

(viii) SEQ ID No: 71 (nucleic acid sequence) and 85 (amino acid sequence)

[SEQ ID No: 71]
GGC TCG CTG CTT GAG AGC CCT AAA GCA TTA GAA GAA GCA CCT TGG CCT CCA CCA GAA GGG TCC
[SEQ ID No: 85]
G S L L E S P K A L E E A P W P P P E G S

Further embodiments of fusion protein were created that lacked the GFP so that cell delivery could be performed with phenotypic cell assays using a Vybrant cell staining kit without interfering fluorescence signals arising from GFP. Variant ferritin fusions were created with different linker amino acid sequences. Hence, these fusion proteins are preferably encoded by a nucleic acid substantially as set out in SEQ ID No:72, 74 and 76, or may comprise an amino acid substantially as set out in SEQ ID No:73, 75 and 77, or fragments or variants thereof.

[SEQ ID No: 72 and 73]
ATG GGC AGC CAT CAC CAT CAC CAC CAT AGC GGC GGT ACG GGC AGC AGC GGT GCC ACT GCA GGT
M G S K K K K K K S G G T G S S G A T A G
GGT AGC CAT AAT AAA TAT AAC AAA CAA CAG CAA AAC GCG TAT TAC GAG AAT CAG CAC CAG
G S D D F P D P D Q Q D A P Y B Y L R L
CCG AAT CAC AAT CAA CAC CAG CGT AAT GCC TTC AAC CAG AGC CAC AAA CAT CAT CCC AGC
P R L R E E Q R R A F T Q D L K D D P G
CAG AGC GCG AAC CTG CTG GCC GAA GCG AAA AAA CTG AAT GAC GCG CAG GCC CCG AAA GTG
Q S A N L L A E A K K L N D A Q A P R V
GAC AAC AAA ATC AAA AAA GAA CAA CAG AAA GCC ATC TAC GAG ATC CTG CAA CTG CCG AAC
Q N K F N K E Q Q N A F Y E I L K L P N
CTG AAA GAA GAA CAG CGC AAA GCC ATA ATC CAG ACC CTG AAA GAA GAA CCG AGC CAG AGC
L N E E Q R N A F T Q G L E D D F G Q G
GCC AAT CTG CTG GCG GAA GCG AAA AAA CTG AAG GAT GCG CAA GCG CCG AAA GTG GGC TCG
A N L L A E A K K L N D A Q A F K V G S
GGC TCA GGC TCC GGA TCT GGT TCA GGT TCA GGA TCG GGC TCC GGG TCC
G S G S G S G S G S G S G S G S
ATG ACC ACG GCG TCT ACT AGC CAG GTC CGC CAA AAC
M T T A S T S Q V R Q N
TAT CAT CAC GAC AGC GAG GCG GCG GCG ATC AAT CGC CAG ATT AAC CTG GAG gcg TAC GCA AGC
Y H Q D S E A A I N R Q I N L E A Y A S
TAC GTT TAC qcq AGC ATG AGC TAC TAT TTC GAT CGC GAT GAC GTT GCG CTG AAA AAC TTC
Y V Y A S M S Y Y P D P D D V A L K N P
GCT AAG TAT TTT CTG CAC CAA AGC CAC GAA GAA CGT GAA CAT GCC GAG AAA CTG ATG AAG
A K Y F L H Q S H E E P E H A E K L M K
CTG CAA AAT CAG CGT GGC GGT CGT gcg TTT gcg CAA GAT ATT AAA AAG CCG GAT TGC GAC
L Q N Q R G G R A F A Q D I K K P D C D
GAC TGG GAA AGC GGC CTG AAC GCA ATG GAG TGT GCG CTG CAC TTG GAG AAA AAC GTG AAT
D W E S G L N A M E C A L H L E K N V N
CAG TCC TTG CTG GAG CTG CAT AAG CTG GCT ACC GAT AAG AAT GAT CCG CAC CTG TGC GAC
Q S L L E L H K L A T D K N D P H L C D
TTC ATT GAA ACG CAC TAT CTG AAT GAA CAG GTG AAG GCA ATC AAA GAA CTG GGT GAT CAC
F I E T H Y L N E Q V K A I K E L G D H
GTC ACC AAT CTG CGT AAA ATG GGT GCC CCG GAG AGC GGC CTG GCG GAG TAC CTG TTT GAC
V T N L P K M G A P E S G L A E Y L F D
AAA CAT ACG TTG GGC GAC TCG GAC AAC GAG TCT Ccc ggg
F H T L G D S D N E S P G
[SEQ ID No: 74 and 75]
ATG GGC AGC CAT CAC CAT CAC CAC CAT AGC CGC GGT ACG GGC AGC AGC GGT GCC ACT GCA GGT
M G S H H H H H H S G G T G S S G A T A G
GGT AGC GAT AAT AAA TTT AAC AAA GAA CAC CAA AAC GCC TTT TAC GAG ATT CTG CAC CTG
G S D N K F N K E Q Q N A F Y E I L R L
GCG AAT CTG AAA GAA GAG CAG CGT AAT GCC TTC ATC CAG ACG CTG AAA GAT GAT CCG AGG
P N L N E E Q R N A F I Q S L K D D P S
CAG AGC GCG AAC CTG CTG GCC GAA GCG AAA AAA CTG AAT GAC GCG CAG GCC CCG AAA GTG
Q S A N L L A E A K K L N D A Q A P K V
GAC AAC AAA TTC AAT AAA GAA CAA CAG AAT GCC TTC TAC GAC ATC CTG CAT CTG CCG AAC
D N K F N K E Q Q N A F Y E I L R L P N
CTG AAT GAA GAA CAG CGC AAT GCC TTT ATC CAG AGC CTG AAA GAT GAT CCG AGC CAG AGC
L D E E Q R D A F I Q S L F D D P S Q S
GCC AAT CTG CTG GCC GAA GCC AAA AAA CTG AAC GAT GCG CAA GCC CCG AAA GTG GGC TCG
A N L L A E A K K L N D A Q A P K V G S
GCC GAA GCG GCT GCT AAA GAA GCA GCT GCT AAA GAG GCT GCC GCC AAG GCA GGG TCC
A E A A A K E A A A K E A A A K A G S
ATG ACC ACG GCG TCT ACT AGC CAG GTC CGC CAA AAC
M T T A S T S Q V R Q N
TAT CAT CAG GAC AGC GAG GCG GCG ATC AAT CGC CAG ATT AAC CTG GAG gcg TAC GCA AGC
Y H Q D S E A A I N R Q I N L E A Y A S
TAC GTT TAC gcg AGC ATG AGC TAC TAT TTC GAT CGC GAT GAC GTT GCG CTG AAA AAC TTC
Y V Y A S M A Y Y F D R D D V A L L K N F
GCT AAG TAT TTT CTG CAC CAA AGC CAC GAA GAA CGT GAA CAT GCC GAG AAA CTG ATG AAG
A K Y F L H Q S H E E R E H A E K L M K
CTG CAA AAT CAG CGT GGC GGT CGT gcg TTT gcg CAA GAT ATT AAA AAG CCG GAT TGC GAC
L Q N Q R G G R A F A Q D I K K P D C D
GAC TGG GAA AGC GGC CTG AAC GCA ATG GAG TGT GCG CTG CAC TTG GAG AAA AAC GTG AAT
D W E S G L N A M E C A L H L E K N V N
CAG TCC TTG CTG GAG CTG CAT AAG CTG GCT ACC GAT AAG AAT GAT CCG CAC CTG TGC GAC
Q S L L E L H K L A T D K N D P H L C D
TTC ATT GAA ACG CAC TAT CTG AAT GAA CAG GTG AAG GCA ATC AAA GAA CTG GGT GAT CAC
F I E T H Y L N E Q V K A I K E L G D H
GTC ACC AAT CTG CGT AAA ATG GGT GCC CCG GAG AGC GGC CTG GCG GAG TAC CTG TTT GAC
V T N L R K M G A P E S G L A E Y L F D
AAA CAT ACG TTG GGC GAC TCG GAC AAC GAG TCT Ccc ggg
K H T L G D S D N E S P G
His tag-ZZ-VARIANT HFTN
[SEQ ID No: 76 and 77]
ATG GGC AGC CAT CAC CAT CAC CAC CAT AGC CGC GGT ACG GGC AGC AGC GGT GCC ACT GCA GGT
M G S H H H H H H S G G T G S S G A T A G
GGT AGC GAT AAT AAA TTT AAC AAA GAA CAC CAA AAC GCC TTT TAC GAG ATT CTG CAC CTG
G S D N K F N K E Q Q N A F Y E I L R L
GCG AAT CTG AAA GAA GAG CAG CGT AAT GCC TTC ATC CAG ACG CTG AAA GAT GAT CCG AGG
P N L N E E Q R N A F I Q S L K D D P S
CAG AGC GCG AAC CTG CTG GCC GAA GCG AAA AAA CTG AAT GAC GCG CAG GCC CCG AAA GTG
Q S A N L L A E A K K L N D A Q A P K V
GAC AAC AAA TTC AAT AAA GAA CAA CAG AAT GCC TTC TAC GAC ATC CTG CAT CTG CCG AAC
D D E F D E E Q Q D A F V E I L F L P D
CTG AAT GAA GAA CAG CGC AAT GCC TTT ATC CAG AGC CTG AAA GAT GAT CCG AGC CAG AGC
L N E E Q R N A F T Q S L F D D P S Q S
GCC AAT CTG CTG GCC GAA GCC AAA AAA CTG AAC GAT GCG CAA GCC CCG AAA GTG GGC TCG
A N L L A E A K K L N D A Q A P K V G S
CTG CTT GAG AGC CCT AAA GCA TTA GAA GAA GCA CCT TGG CCT CCA CCA GAA GCG TCC
L L E S P K A L E E A P W P P P E G S
ATG ACC ACG GCG TCT ACT AGC CAG GTC CGC CAA AAC
M T T A S T S Q V R Q N
TAT CAT CAG GAC AGC GAG GCG GCG ATC AAT CGC CAG ATT AAC CTG GAG gcg TAC GCA AGC
Y H Q D S E A A I N R Q I N L E A Y A S
TAC GTT TAC gcg AGC ATG AGC TAC TAT TTC GAT CGC GAT GAC GTT GCG CTG AAA AAC TTC
Y V Y A S M S Y Y F D R D D V A L L K N F
GCT AAG TAT TTT CTG CAC CAA AGC CAC GAA GAA CGT GAA CAT GCC GAG AAA CTG ATG AAG
A K Y F L H Q S H E E R E H A E K L M K
CTG CAA AAT CAG CGT GGC GGT CGT gcg TTT gcg CAA GAT ATT AAA AAG CCG GAT TGC GAC
L Q N Q R G G R A F A Q D I K K P D C D
GAC TGG GAA AGC GGC CTG AAC GCA ATG GAG TGT GCG CTG CAC TTG GAG AAA AAC GTG AAT
D W E S G L N A M E C A L H L E K N V N
CAG TCC TTG CTG GAG CTG CAT AAG CTG GCT ACC GAT AAG AAT GAT CCG CAC CTG TGC GAC
Q S L L E L H K L A T D K N D P H L C D
TTC ATT GAA ACG CAC TAT CTG AAT GAA CAG GTG AAG GCA ATC AAA GAA CTG GGT GAT CAC
F I E T H Y L N E Q V K A I K E L G D H
GTC ACC AAT CTG CGT AAA ATG GGT GCC CCG GAG AGC GGC CTG GCG GAG TAC CTG TTT GAC
V T N L R K M G A P E S G L A E Y L F D
AAA CAT ACG TTG GGC GAC TCG GAC AAC GAG TCT Ccc ggg
K H T L G D S D N E S P G

As shown in the Examples, the variant ferritin polypeptides developed by the inventors have been mutated in such a way that they cannot self-assemble to form a nanocage unless they have been contacted with a nucleating agent, such as a metallic (e.g. gold) nanoparticle, in which case the mutant self-assembles around the metallic core, thereby forming a nanocage and encapsulating the core.

In a further aspect, there is provided an isolated nucleic acid comprising or consisting of a nucleotide sequence encoding the variant ferritin polypeptide of the first aspect or the fusion protein of the second aspect, or a fragment or variant thereof.

The nucleic acid preferably comprises or consists of one or more of the nucleotide sequences described herein. Preferred nucleic acids comprise or consist of a nucleotide sequence substantially as set out in any one of SEQ ID No: 5, 9, 11, 30, 32, 36, 38, 40, 42, 44, 46, 50, 52, 54, 56, 58, 60 or 62.

Thus, in a third aspect, there is provided a ferritin nanocage comprising the variant ferritin polypeptide of the first aspect or the fusion protein of the second aspect, and a nucleating agent.

In one embodiment, the nanocage may comprise a plurality of identical monomers of ferritin polypeptide or fusion protein. For example, in one embodiment, each monomer may comprise ferritin, and one or more domain selected from a group consisting of: an antibody or antigen binding fragment thereof binding peptide; a fluorophore; a His tag; and a nucleating agent binding peptide. Preferably, the monomer comprises human ferritin, optionally the light chain or heavy chain ferritin. For example, as described in Example 13, the monomer may comprise His-ZZ-hFtn(L29A L36A L81A L83A). Thus, the resultant nanocage will contain the ZZ domain and the GFP domain on each subunit. It will be appreciated that other combinations of domain can be included in the monomer, which is used throughout the nanocage, such that the same domains are presented in each subunit of the nanocage.

However, in another embodiment, the nanocage may comprise a plurality of different monomers of ferritin polypeptide or fusion protein. For example, the nanocage may comprise first and second monomers comprising ferritin, and one or more domain selected from a group consisting of: an antibody or antigen binding fragment thereof binding peptide; a fluorophore; a His tag; and a nucleating agent binding peptide, wherein the first and second monomers have different combinations of domains. Preferably, the monomer comprises human ferritin, optionally the light chain or heavy chain ferritin.

As described in Example 13, compound or mixed nanocages composed of different types of ferritin subunit were also created. For example, in one embodiment, a first monomer may comprise His-ZZ-hFtn(L29A L36A L81A L83A) and a second monomer may comprise His-GFP-hFtn(L29A L36A L81A L83A). Since the hFtn part of the resultant fusion protein is identical, nanocages form that contain the ZZ domain on some subunits, and the GFP domain on others. It will be appreciated that other combinations of domain can be included in a variety of different monomers present in the nanocage, such that the different domains are presented in the subunits of each nanocage.

In a fourth aspect, there is provided a method of preparing a ferritin nanocage, the method comprising contacting the variant ferritin polypeptide of the first aspect or the fusion protein of the second aspect, with a nucleating agent.

The nucleating agent preferably comprises a nanoparticle having an average diameter of about 1-500 nm, more preferably 1-100 nm, even more preferably 2-50 nm, and most preferably 3-10 nm. Preferably, the nucleating agent is metallic. For example, the nucleating agent may be gold, iron, or copper. In an alternative embodiment, the agent may comprise a gadolinium binding peptide.

Preferably, the ferritin polypeptide encapsulates the nucleating agent. Most preferably, the ferritin nanocage encapsulates a gold nanoparticle.

Advantageously, the method according to the invention can be used to easily create a ferritin nanocage. Furthermore, the method according to the invention does not require the use of harsh denaturation conditions in order to create a nanocage, which is advantageous because it reduces the likelihood of destroying the integrity of the reformed nanocage.

The inventors have also shown that the nanocage can be modified to be fluorescent by fusion of an N-terminal fluorescent protein to the ferritin monomer, for use in diagnostics and imaging experiments. Thus, preferably the ferritin nanocage is functionalised with an imaging agent, such as a fluorescent protein or fluorophore. The nanocages of the invention can be modified to become fluorescent by fusion or conjugation of a fluorescent protein, for example GFP or the like. Preferably, the fluorescent protein is fused at or towards the N-terminus of the ferritin polypeptide.

Furthermore, the inventors have also demonstrated that the nanocage can be “decorated” with antibodies, and thereby targeted to cells by further fusion of an antibody binding domain, so that antibody-bound nanocage can specifically bind to target cells. Preferably, the antibody binding domain is fused to the N-terminus of the ferritin monomer. Advantageously, specific targeting and endocytosis of the nanocage can be achieved by modifying the ferritin with an IgG binding domain. This enables the nanocage to bind to IgG type antibodies in a simple binding reaction. Thus, binding of the ferritin nanocage to an antibody leads to specific targeting of cells. Furthermore, by using an antibody that targets endocytic receptors, such as the EGFR receptor, the nanocage can be endocytosed (Goh & Sorkin, CSH Perspect. Biol. 5(5), 2013), which leads to delivery of the nanocage and its contents directly into the cell. As described in Examples 11 and 12, the nanocage of the invention has been successfully functionalised with anti-EGFR antibodies.

Preferably, therefore, the ferritin nanocage comprises, or is functionalised with an antibody or antigen binding fragment thereof. Preferably, the antibody or antigen binding fragment thereof is immunospecific for endocytic receptors. As such, the nanocage is endocytosed leading to delivery of the nanocage and its contents directly into the target cell.

A preferred antibody or antigen binding fragment thereof binding amino acid sequence comprises a Z-domain, which is a derivative of Staphylococcus protein A. This is an engineered version of the IgG binding domain of protein A with greater stability and a higher binding affinity for the Fc antibody domain. Accordingly, preferably the ferritin nanocage is functionalised with an IgG antibody. Preferably, the ferritin nanocage is functionalised by binding to the Fc domain of the antibody, so that antigen recognition is not compromised through direct interaction with the Fv domain. The antibody or antigen binding fragment thereof preferably exhibits immunospecificity for a target cell or tissue. Thus, the nanocage can be targeted to specific cells (e.g. a tumour cell) by fusion of an antibody binding domain at or towards the N-terminus of the ferritin polypeptide. Advantageously, therefore, functionalised nanocages according to the invention can be targeted to specific cells, and simultaneously visualised.

The inventors have therefore realised that the nanocage of the invention can be used as a vector for delivering drug molecules to a target cell or tissue.

Hence, in yet a further aspect, there is provided a ferritin nanocage according to invention, for use as a vector for the delivery of a payload molecule, preferably a drug molecule, to a target biological environment.

The nucleating agent, which is preferably a metallic nanoparticle, may be bound to a payload which may be an active agent, such as a drug molecule. Thus, preferably the ferritin nanocage is configured, in use, to encapsulate and carry the payload molecule to a target biological environment. The nanocage comprises an internal cavity in which the payload molecule is contained, wherein the payload molecule is capable of being active when the nanocage is at least adjacent to the target biological environment.

Thus, in a fifth aspect, there is provided a method of encapsulating a payload molecule, preferably a drug molecule, in a ferritin nanocage, the method comprising contacting the variant ferritin polypeptide of the first aspect or the fusion protein of the second aspect with a nucleating agent conjugated to a payload molecule and allowing the polypeptide or protein to self-assemble into a nanocage, thereby encapsulating the payload molecule.

The payload molecule described herein may be an active agent, such as a small molecule drug, which may be bound to the nucleating agent prior to encapsulation and subsequent mixing of the variant ferritin polypeptide or fusion protein. The molecular weight of the payload molecule may be 50 Da to 10 kDa, preferably 100 Da to 1 kDa, more preferably 250 Da to 1000 Da.

The anti-cancer drug doxorubicin was used as an exemplary active agent in the Examples, and is therefore most preferred. Another preferred payload molecule is paclitaxel, as described in Example 11. The payload molecule may be an antibiotic, such as actinomycin, as described in Example 12. The payload molecule may therefore be a peptide, or cyclic peptide. Yet another preferred payload molecule is actinomycin-D. As described in Example 14, using mass spectrometry, 13.3 actinomycin D molecules have been encapsulated by the nanocage.

The payload molecule may be bound or conjugated to the nucleating agent by van der Waal's forces or ionic forces. The nucleating agent-drug conjugate leads to the formation of the ferritin nanocage which encapsulates the nucleating agent and the active agent conjugates thereto within the nanocage. Advantageously, therefore the method according to the invention can be used to easily load a drug into a ferritin nanocage. A further advantage of the invention is that it can be used to widen the therapeutic window of drugs that are otherwise incapable of permeating cells without assistance. Preferably, the nucleating agent is a metallic nanoparticle, more preferably a gold nanoparticle.

The inventors have generated an innovative approach to producing and using ferritin as a targetable drug delivery agent. They have engineered mutations in the ferritin monomer so that it does not form a nanocage in isolation, and can be purified in its monomeric state. When mixed with a metallic nanoparticle, the nanoparticle acts as a nucleation site and the nanocage specifically reforms around the metallic nanoparticle. Functionalising the nanocage with a suitable antibody ensures that the nanocage is targeted to a target site. Example 5 explains how the nanocage can be targeted to MNK1.1 (mouse natural killer cells) and HT29 (colorectal cancer) cell lines, which have known antibodies that can either target the NK1.1 receptor in the case of MNK1.1, or the EGFR receptor in the case of HT29.

In a sixth aspect, there is provided a method of targeting a ferritin nanocage to a target biological environment, the method comprising functionalising the ferritin nanocage of the third aspect with an antibody or antigen binding fragment thereof which is immunospecific for a target cell, and allowing the functionalised nanocage to be targeted to the target biological environment.

The ability to target ferritin nanocages to specific cell types via the binding of antibodies creates huge possibilities for the diagnosis and treatment of disease. When the ferritin nanocage reaches the desired target biological environment, it is subjected to a decrease in pH associated with lysosomes, which causes the otherwise encapsulated payload molecule agent to be released from the nanocage, where it then exerts its biological effect.

Because the nanocages can be made fluorescent, they can be used in imaging methods to identify specific cell types displaying known epitope disease markers. This creates possibilities for their use in the diagnosis of cancer types in imaging accessible locations. Thus, the target biological environment may be a cell or tissue, such as a cancer or tumour cell. Examples are cancers accessible via GI-tract, such as oesophageal, stomach, colorectal, liver, pancreatic, gall bladder. In addition, cancers near to the surface of the body would be accessible for diagnosis including skin cancer and neck and throat cancers.

Furthermore, because the drug-encapsulated complex contains a metallic (e.g. gold) nanoparticle, a mechanism for the activated release of drugs is also possible. Gold nanoparticles absorb light due to their plasmonic effect and laser irradiation may be used to cause localised heating of the nanoparticle proportional to the intensity of the incident laser irradiation. Following targeting of the nanocage to its target biological environment, laser induced heating may therefore be used to activate the release of the encapsulated drug, since localised heating will lead to the thermal disassembly of the nanocage complex in the same way that the pH drop associated with endosomes does. This type of approach can make use of current endoscope technology that can both locally deliver compounds, image and treat using laser light sources. The inventors therefore consider that this type of nanocage device would fit with current therapeutic practices and approaches.

The ability to encapsulate drugs into the nanocage also provides the possibility of combined diagnostic and therapy (theranostic) approaches.

Accordingly, in a seventh aspect, there is provided the variant ferritin polypeptide of the first aspect, the fusion protein of the second aspect or the ferritin nanocage of the third aspect, for use in therapy or diagnosis.

In an eighth aspect, there is provided the variant ferritin polypeptide of the first aspect, the fusion protein of the second aspect or the ferritin nanocage of the third aspect, for use in the treatment, prevention or amelioration of disease, preferably cancer.

In a ninth aspect, there is provided a method of treating, ameliorating or preventing a disease, preferably cancer, the method comprising administering, to a subject in need of such treatment, a therapeutically effective amount of the variant ferritin polypeptide of the first aspect, the fusion protein of the second aspect or the ferritin nanocage of the third aspect.

Preferably, the method comprises administering the ferritin nanocage of the third aspect to the subject, and then exposing the nanocage to heat such that it disassembles, thereby releasing the payload molecule.

The heat may be provided by a suitable heat source, such as a laser. The principle of laser-induced drug release has been demonstrated by examining the fluorescence polarisation of a fluorescently-bound molecule within the nanocage, such as Dox. Anisotropy provides an intensity independent measure of the degree of polarisation within a sample. When a fluorescent molecule absorbs plane polarised light, it will be emitted in the same plane as the excitation source. However, during the fluorescence lifetime, between absorption and emission, the molecule may rotate. This means that the emitted light will be relative to the new orientation of the molecule. By measuring the emitted light in both vertical and horizontal planes, it is possible to determine the degree of polarisation (anisotropy). Because large molecules rotate slower than small molecules, the degree of anisotropy will be dependent on the size of the molecule. A fluorescent molecule encapsulated in the nanocage will therefore have a very high anisotropy value. Laser irradiation of the metallic nanoparticle leads to the breakdown of the nanocage and release of a fluorescent compound, and this can be imaged by a significant reduction in the measured anisotropy.

Hence, in a tenth aspect, there is provided use of a heat source to heat a ferritin nanocage according to the third aspect comprising an encapsulated payload molecule, to disassemble the nanocage and thereby release the payload molecule.

The heat source may be a laser.

The inventors also believe that the nanocage can be used in phenotypic screens for use in drug development.

Thus, in an eleventh aspect, there is provided use of the ferritin nanocage according to the third aspect to correlate drug delivery to a cell with its therapeutic effect.

In a twelfth aspect, there is provided a phenotypic assay comprising the ferritin nanocage according to the third aspect.

For example, the inventors have demonstrated the ability to use the ferritin nanocage as a platform technology for the delivery of small molecule drugs into cells. Because the technology provides a defined process for the encapsulation and assembly of the nanocage complex, it can be envisioned as a generic method for the delivery of compounds into cells. The binding of small molecule compounds to the metallic nanoparticle core would work for a wide variety of ionic, electrostatic and hydrophobic interactions. The assembly of the mutant nanocage around the drug-bound nanoparticle also appears robust. Further, the binding of the nanocage complex to an antibody by interaction of the ZZ domain with IgG isotype antibodies is fast and effective. This can therefore be applied to a very wide range of commercially available antibodies and so can be used to effectively target a wide range of different cell types.

Because of the ordered process and versatility of nanocage delivery, it is possible to use this as a platform for screening small molecules for in vivo efficacy. In many instances small molecule drugs fail because of poor cell permeability. Furthermore, during drug development conclusions are frequently made regarding efficacy of classes of compounds in phenotypic cell assays but without any knowledge of cell permeability; the drugs may be highly effective if they can be made to cross the cell membrane. Being able to further delineate the mode of failure, non-cell penetration, or poor biological effectiveness, would be valuable in screening campaigns.

The ferritin nanocage of the invention provides a methodology for the effective delivery of compounds into cells in a phenotypic assay and the ordered assembly process is adaptable to high throughput screening scenarios. Furthermore, nanocages that are made fluorescent, either through chemical labelling, or the fusion of fluorescent proteins, can be used to monitor the uptake of individual cells. When combined with cell sorting methods the phenotypic assays could be correlated to a dose response based on the nanocage fluorescence.

For example, the inventors have used phenotypic assays to demonstrate the effective delivery of the active agent Dox into cells. The MTT assay measures the metabolic activity of cells via NAD(P)H dependent oxidoreductase enzymes using a tetrazolium dye substrate (MTT) that produces a purple colour on reduction. A reduced numbers of viable cells leads to a loss of activity and hence a reduced colour response. For example, the variant ferritin polypeptides described herein may be used to create nanocages encapsulating the test drug. In the case of the Dox loaded nanocages, two concentrations of Dox (0.1 μM & 0.2 μM) may be used when forming the complexes. They may be mixed with anti-EGFR and their interaction with HT29 cells may be monitored over time prior to measuring viability using the MTT assay. The nanocages that were formed with the higher loading of Dox should demonstrate a phenotypic response during the time course of the assay. The data should also demonstrate a dose response to the different nanocage loading conditions used of Dox (0.1 or 2.0 μM).

A further phenotypic assay may be performed using flow cytometry and a suitable dye, such as the Topro3 dye. Topro3 binds to DNA and preferentially enters non-viable cells. As before, HT29 cells may be treated with Au-ZZ-GFP-hFTN (L29A L36A I81A L83A) and Dox-Au-ZZ-hFTN (L29A L36A I81A L83A) complexes pre-bound to the anti-EGFR antibody. A control of Dox only may also performed along with cells only.

It will be appreciated that the variant ferritin polypeptide of the first aspect, the fusion protein of the second aspect or the ferritin nanocage according to the third aspect (i.e.

which is referred to hereinafter as “agent” or “active agent”) may be used in a medicament which may be used in a monotherapy, or as an adjunct to, or in combination with, known therapies for treating, ameliorating, or preventing disease, such as cancer.

The agents according to the invention may be combined in compositions having a number of different forms depending, in particular, on the manner in which the composition is to be used. Thus, for example, the composition may be in the form of a powder, tablet, capsule, liquid etc. or any other suitable form that may be administered to a person or animal in need of treatment. It will be appreciated that the vehicle of medicaments according to the invention should be one which is well-tolerated by the subject to whom it is given.

Medicaments comprising the agents according to the invention (i.e. the ferritin nanocage) may be used in a number of ways. For instance, oral administration may be required, in which case the agents may be contained within a composition that may, for example, be ingested orally in the form of a tablet, capsule or liquid. Compositions comprising agents of the invention may be administered by inhalation (e.g. intranasally). Compositions may also be formulated for topical use. For instance, creams or ointments may be applied to the skin.

Agents according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with agents used according to the invention is required and which would normally require frequent administration (e.g. at least daily injection).

In a preferred embodiment, agents and compositions according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the ferritin nanocage that is required is determined by its biological activity and bioavailability, which in turn depends on the mode of administration, the physiochemical properties of the active agent it encapsulates, if present, and whether it is being used as a monotherapy, or in a combined therapy. The frequency of administration will also be influenced by the half-life of the agent within the subject being treated. Optimal dosages to be administered may be determined by those skilled in the art, and will vary with the particular agent in use, the strength of the pharmaceutical composition, the mode of administration, and the advancement of the disease. Additional factors depending on the particular subject being treated will result in a need to adjust dosages, including subject age, weight, gender, diet, and time of administration.

Generally, a daily dose of between 0.01 μg/kg of body weight and 500 mg/kg of body weight of the nanocage and/or active agent according to the invention may be used. More preferably, the daily dose is between 0.01 mg/kg of body weight and 400 mg/kg of body weight, and more preferably between 0.1 mg/kg and 200 mg/kg body weight.

As discussed in the Examples, the ferritin nanocage may be administered before, during the or after the onset of disease. For example, the nanocage may be administered immediately after a subject has developed a disease. Daily doses may be given systemically as a single administration (e.g. a single daily injection). Alternatively, the nanocage may require administration twice or more times during a day. As an example, nanocage may be administered as two (or more depending upon the severity of the disease being treated) daily doses of between 25 mg and 7000 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of nanocage according to the invention to a patient without the need to administer repeated doses.

Known procedures, such as those conventionally employed by the pharmaceutical industry (e.g. in vivo experimentation, clinical trials, etc.), may be used to form specific formulations comprising the nanocage according to the invention and precise therapeutic regimes (such as daily doses of the nanocage and/or active agent and the frequency of administration).

Hence, in a thirteenth aspect of the invention, there is provided a pharmaceutical composition, comprising the variant ferritin polypeptide of the first aspect, the fusion protein of the second aspect or the ferritin nanocage of the third aspect, and a pharmaceutically acceptable vehicle.

The composition can be used in the therapeutic amelioration, prevention or treatment of any disease in a subject that is treatable, such as cancer.

The invention also provides, in an fourteenth aspect, a process for making the pharmaceutical composition according to the thirteenth aspect, the process comprising contacting a therapeutically effective amount of the variant ferritin polypeptide of the first aspect, the fusion protein of the second aspect or the ferritin nanocage of the first aspect, and a pharmaceutically acceptable vehicle.

A “subject” may be a vertebrate, mammal, or domestic animal. Hence, agents, compositions and medicaments according to the invention may be used to treat any mammal, for example livestock (e.g. a horse), pets, or may be used in other veterinary applications. Most preferably, however, the subject is a human being.

A “therapeutically effective amount” of agent is any amount which, when administered to a subject, is the amount of drug that is needed to treat the target disease, or produce the desired effect, e.g. result in tumour killing.

For example, the therapeutically effective amount of nanocage and/or active agent used may be from about 0.01 mg to about 800 mg, and preferably from about 0.01 mg to about 500 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the nanocage may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The nanocage may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The nanocage may be prepared as a sterile solid composition that may be dissolved or suspended at the time of administration using sterile water, saline, or other appropriate sterile injectable medium.

The nanocage and pharmaceutical compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The nanocage according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The skilled technician will appreciate that in order to calculate the percentage identity between two DNA/polynucleotide/nucleic acid sequences, an alignment of the two sequences must first be prepared, followed by calculation of the sequence identity value. The percentage identity for two sequences may take different values depending on: (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two

DNA/polynucleotide/nucleic acid sequences is then calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula: Sequence Identity=(N/T)*100.

Alternative methods for identifying similar sequences will be known to those skilled in the art. For example, a substantially similar nucleotide/nucleic acid sequence will be encoded by a sequence which hybridizes to the sequences shown in any one of SEQ ID Nos. 1 to 10, or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C.

Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

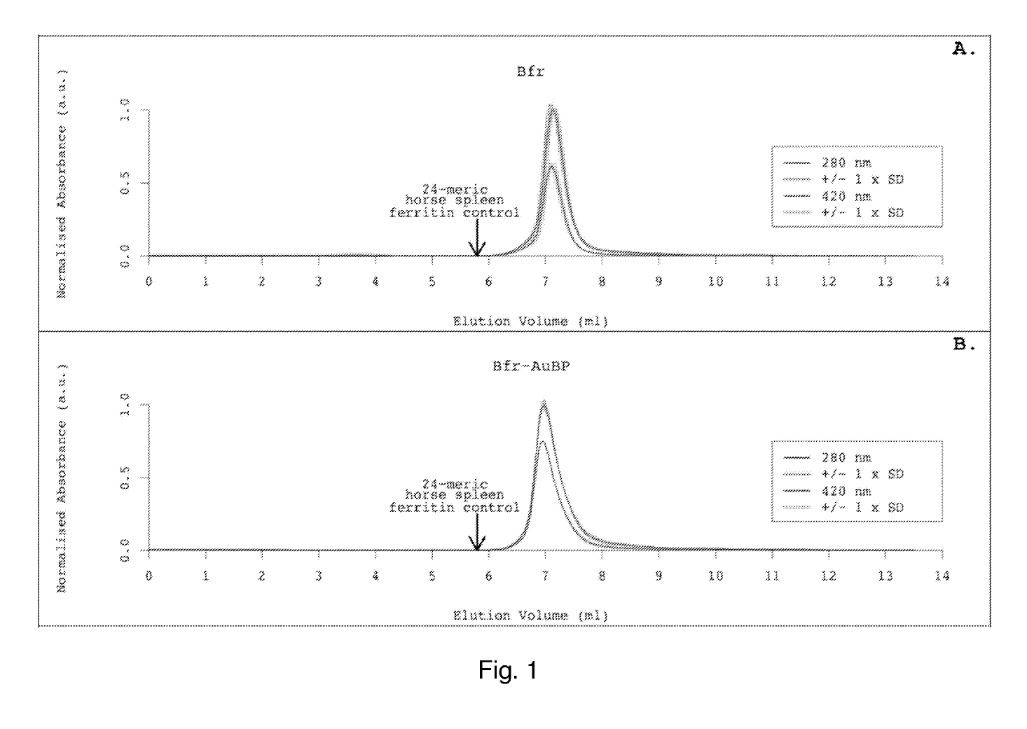

FIG. 1 shows the results of size exclusion of Bfr. (A.) SEC trace for Bfr with elution peak at 7.13 ml. (B.) SEC trace for Bfr-AuBP with elution peak at 6.97 ml. The black arrow that intersects the x-axis at 5.79 ml shows the elution point of commercial 24-meric horse spleen ferritin. The dark blue and red lines correspond to the absorbance readings at 280 nm and 420 nm respectively. The light blue and red shading corresponds to ±1 standard deviation of the mean absorbance readings at 280 nm (protein) and 420 nm (heme), respectively. Each data set is composed of three biological repeats;

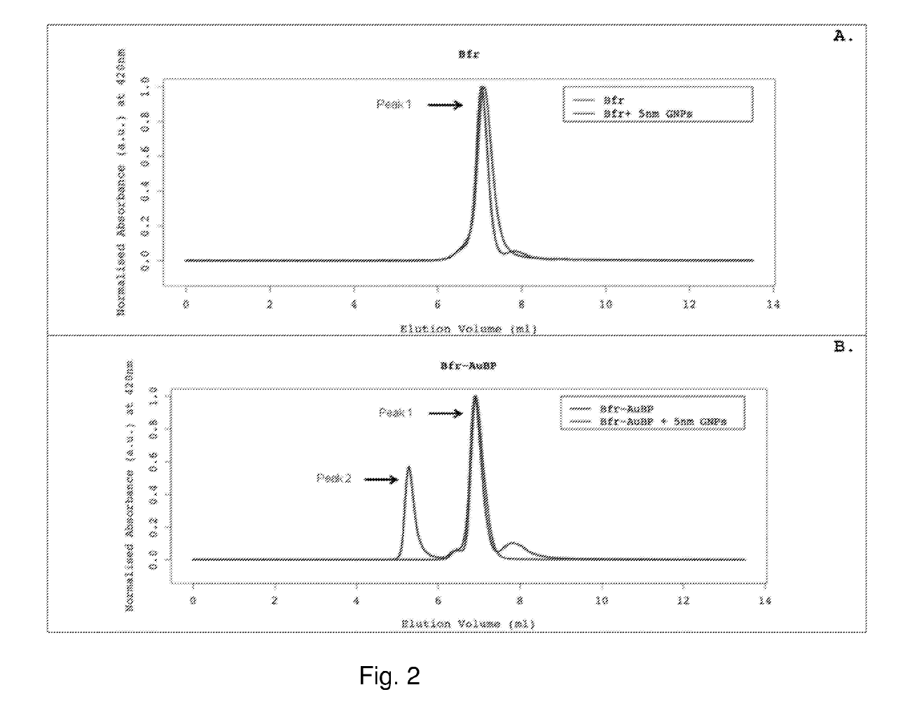

FIG. 2 shows the results of size exclusion chromatography of Bfr with Au nanoparticle. (A) SEC traces for Bfr with and without GNPs shown in red and blue respectively. (B) SEC traces for Bfr-AuBP with and without GNPs shown in red and blue respectively. Peak 1 is the ferritin monomer or dimer, and peak 2 is the 24-mer nanocage. This demonstrates separation of monomer/dimer from nanocage;

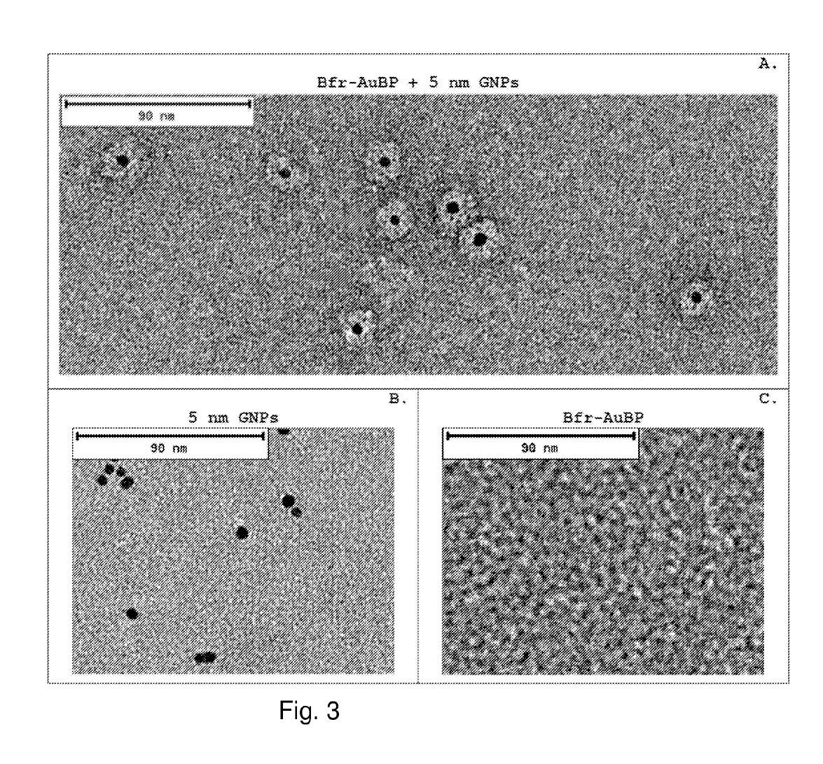

FIG. 3 shows the results of TEM of Bfr with AuNP. (A) Micrograph of Peak 2 (FIG. 2B) showing eight hybrid nanoparticles one of which is highlighted by a blue arrow. The GNPs appear as black circles. The Bfr-AuBP protein component appears as a light halo around each of the encapsulated AuNPs (black circles). A possible protein aggregate is highlighted with a red arrow. (B) Micrograph showing naked GNPs as a control. (C) Micrograph of Peak 1 (FIG. 2B) showing Bfr-AuBP in the absence of AuNPs;

FIG. 4 shows dimeric interfaces in light chain ferritin (lFTN) and heavy chain ferritin (hFTN). A.lFTN dimer (PDB ID:2FG8 (asymmetric unit) [156]). B. hFTN dimer (PDB ID: 3AJO (biological assembly 1) [158]). For each dimer, one subunit is shown in orange and the other is shown in blue. C.lFTN dimer highlighting the conserved hydrophobic residues in the dimer interface and the list of mutations. D. hFTN dimer highlighting the conserved hydrophobic residues in the dimer interface. E. conserved motifs at the dimer interface for light chain and heavy chain ferritin (lFTN and hFTN) that contain hydrophobic residues and the mutations associated with these conserved domains;

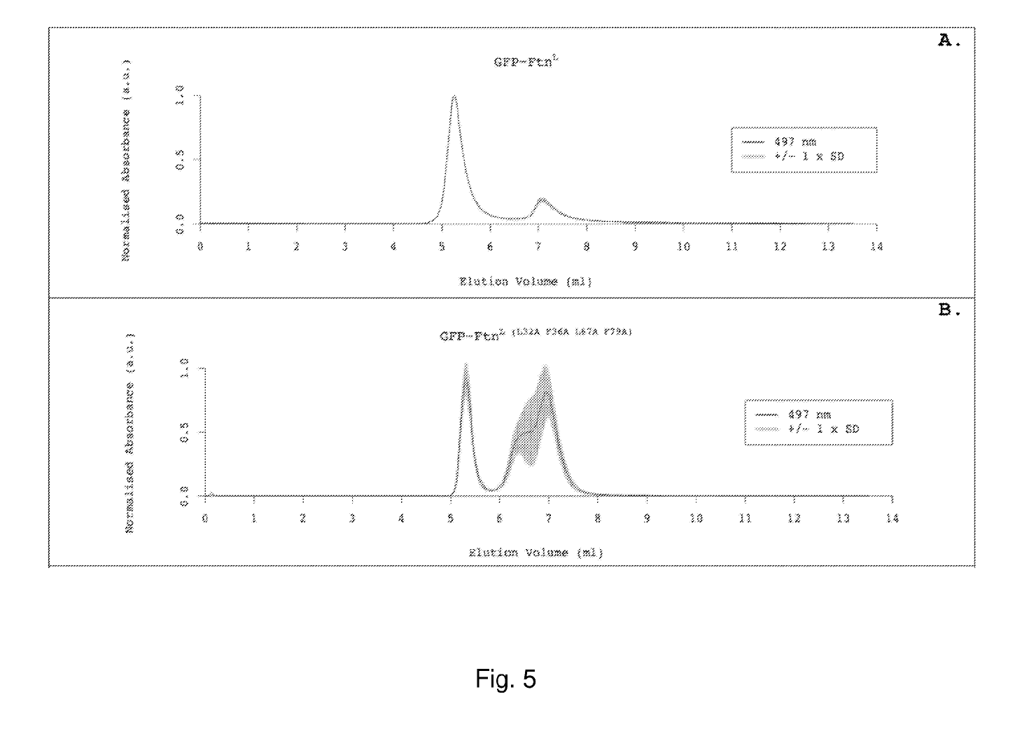

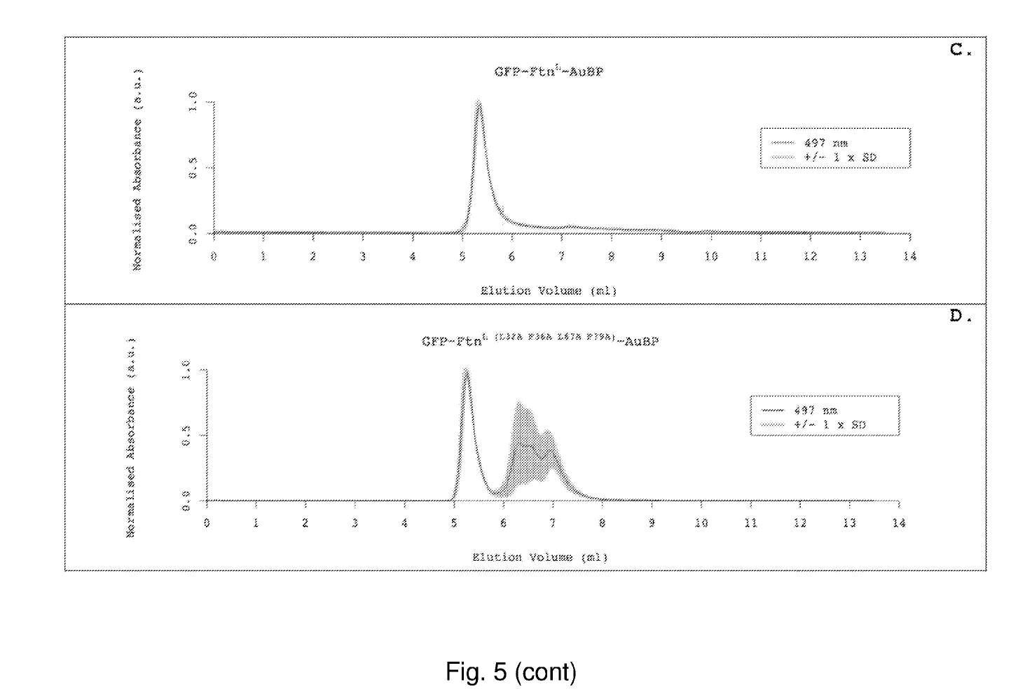

FIG. 5 the results of destabilisation of lFTN variants by mutagenesis. HPLC SEC chromatograms of (A.) GFP-lFTN, (B.) GFP-lFTN (L32A F36A L67A F79A), (C.) GFP-lFTN-AuBP and (D.) GFP-lFTN (L32A F36A L67A F79A)-AuBP. In all chromatograms, the 24-mer elutes at approximately 5.3 ml and the monomer elutes at approximately 7.1 ml. Constructs containing a mutated version of the hFTN subunit (lFTN (L32A F36A L67A F79A) are seen to elute with a lower proportion of nanocage (panels B. & D.), although a significant degree of 24-mer cage remains and a number of other bands are seen that do not coincide directly with monomer and may be assembly intermediates (>1 and <24 subunits). The dark green line corresponds to the absorbance readings at 497 nm (GFP absorbance). The light green shading corresponds to ±1 standard deviation of the mean absorbance readings at 497 nm. Each dataset is comprised of three biological repeats;

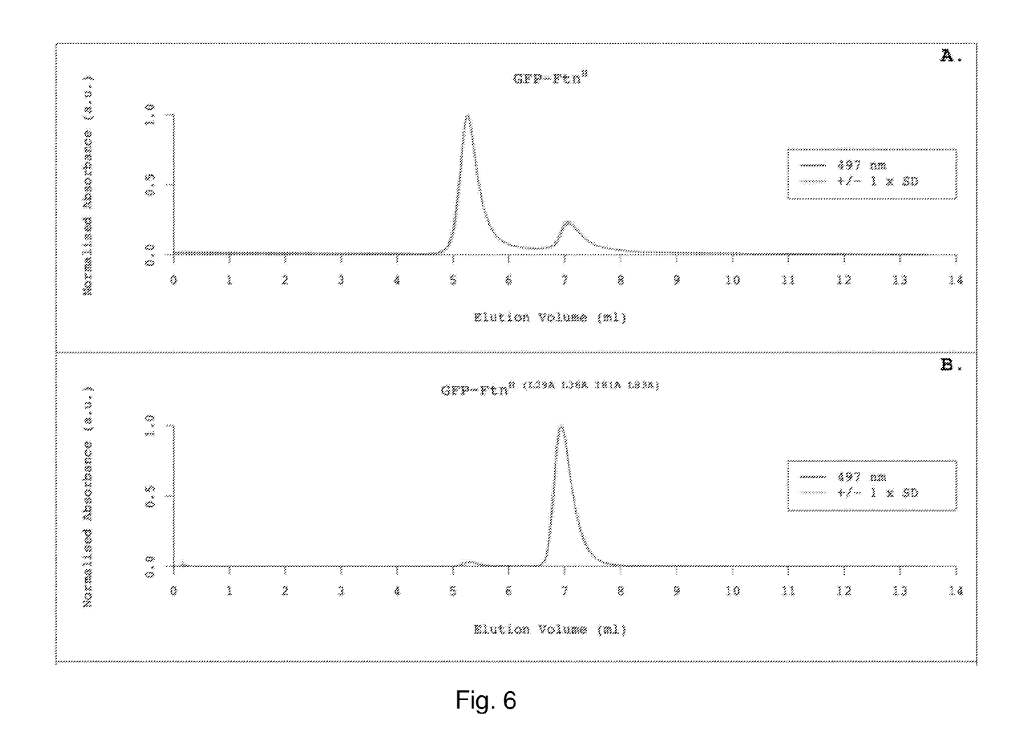

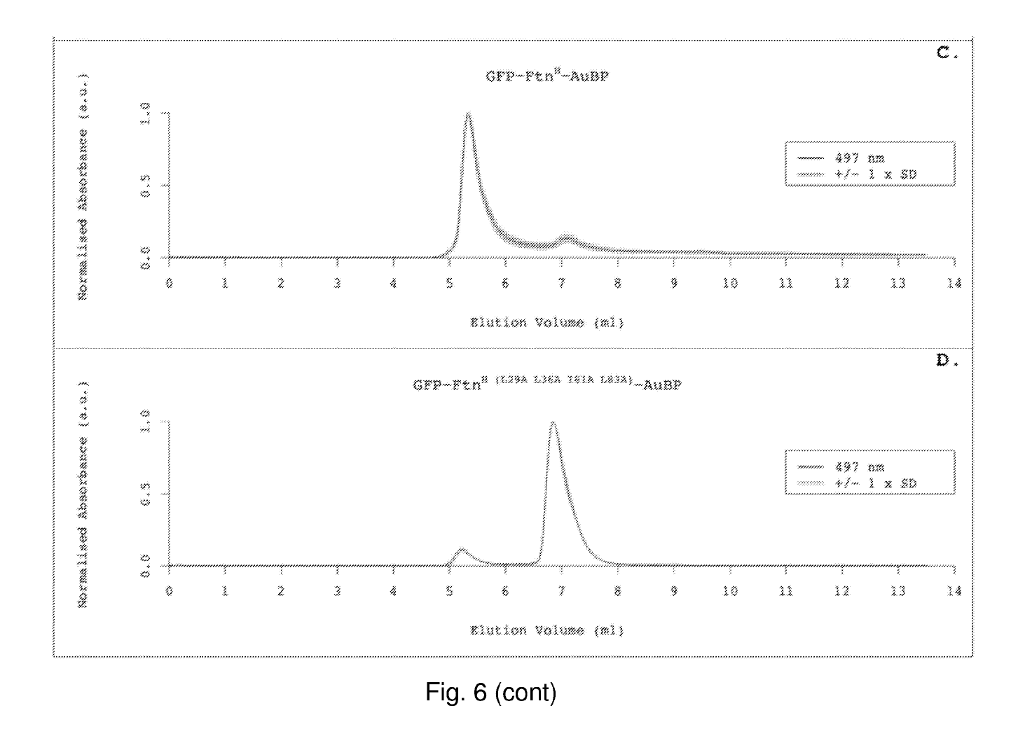

FIG. 6 shows the results of hFTN variants by mutagenesis. HPLC SEC chromatograms of (A.) GFP-hFTN, (B.) GFP-hFTN (L29A L36A I81A L83A), (C.) GFP-hFTN-AuBP and (D.) GFP-hFTN (L29A L36A I81A L83A)-AuBP. In all chromatograms, the 24-mer elutes at approximately 5.3 ml and the monomer elutes at approximately 7.1 ml. Constructs containing a mutated version of the hFTN subunit (hFTN (L29A L36A I81A L83A) are seen to elute primarily as monomers (panels B. & D.) The dark green line corresponds to the absorbance readings at 497 nm (GFP absorbance). The light green shading corresponds to ±1 standard deviation of the mean absorbance readings at 497 nm. Each dataset is comprised of three biological repeats;

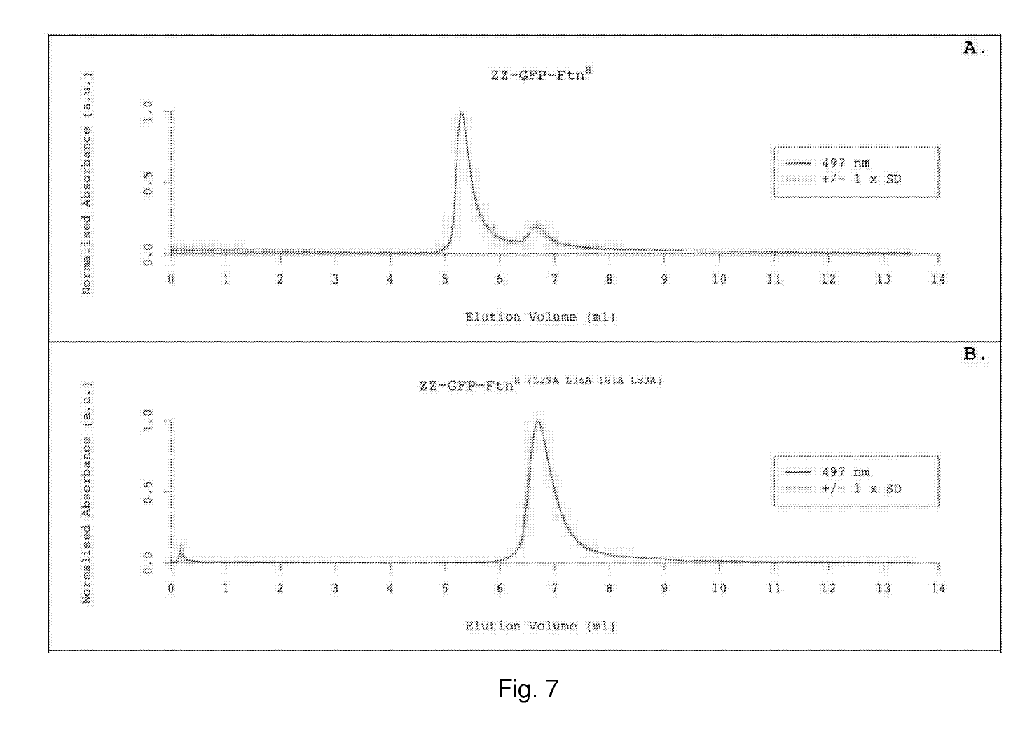

FIG. 7 shows ZZ-GFP fusions of hFTN. HPLC SEC chromatograms of (A.) ZZ-GFP-hFTN, (B.) ZZ-GFP-hFTN (L29A L36A I81A L83A). In all chromatograms, the 24-mer elutes at approximately 5.3 ml and the monomer elutes at approximately 6.9 ml. The ZZ-GFP fusion with wt hFTN is seen to elute primarily as 24-mer (panel A), while the mutated hFTN (L29A L36A I81A L83A) is seen to elute primarily as monomer (panel B) The dark green line corresponds to the absorbance readings at 497 nm (GFP absorbance). The light green shading corresponds to ±1 standard deviation of the mean absorbance readings at 497 nm. Each dataset is comprised of three biological repeats;

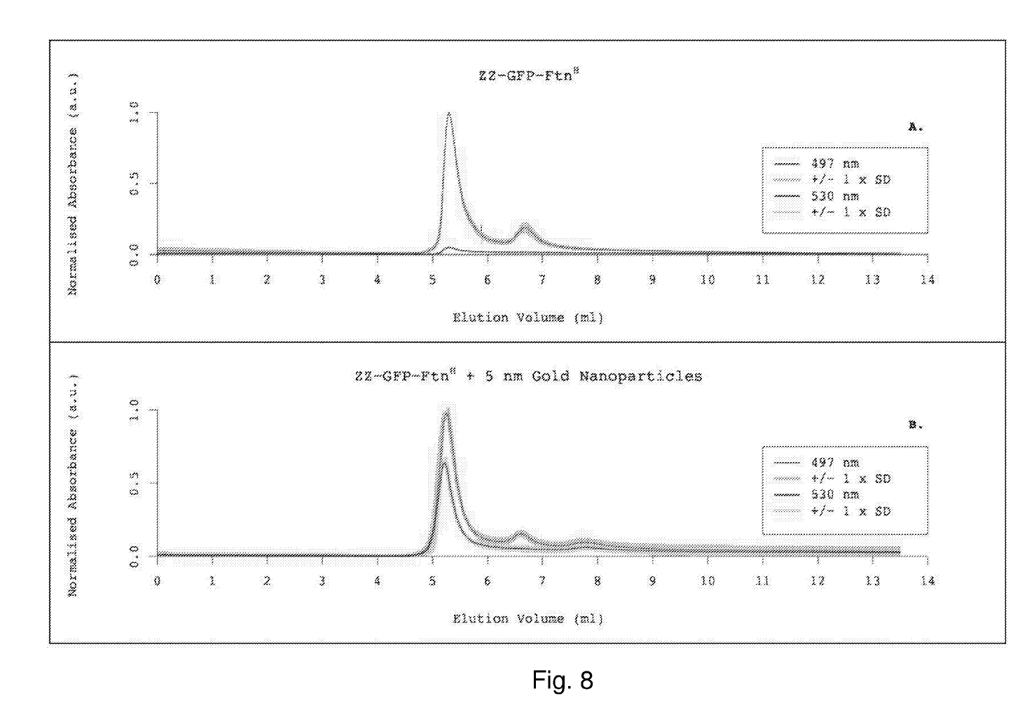

FIG. 8 shows behaviour of hFTN. HPLC SEC chromatograms of (A.) ZZ-GFP-hFTN, (B.) ZZ-GFP-hFTN with AuNP. In all chromatograms, the 24-mer elutes at approximately 5.3 ml and the monomer elutes at approximately 6.8 ml. The wt hFTN is seen to elute primarily as 24-mer (panel A). In the presence of AuNP, the AuNP co-elutes with the FTN 24-mer. The dark green line corresponds to the absorbance readings at 497 nm (GFP absorbance) and the dark blue line absorbance at 530 nm (AuNP absorbance). The shading in both instances corresponds to ±1 standard deviation of the mean absorbance readings. Each dataset is comprised of three biological repeats;

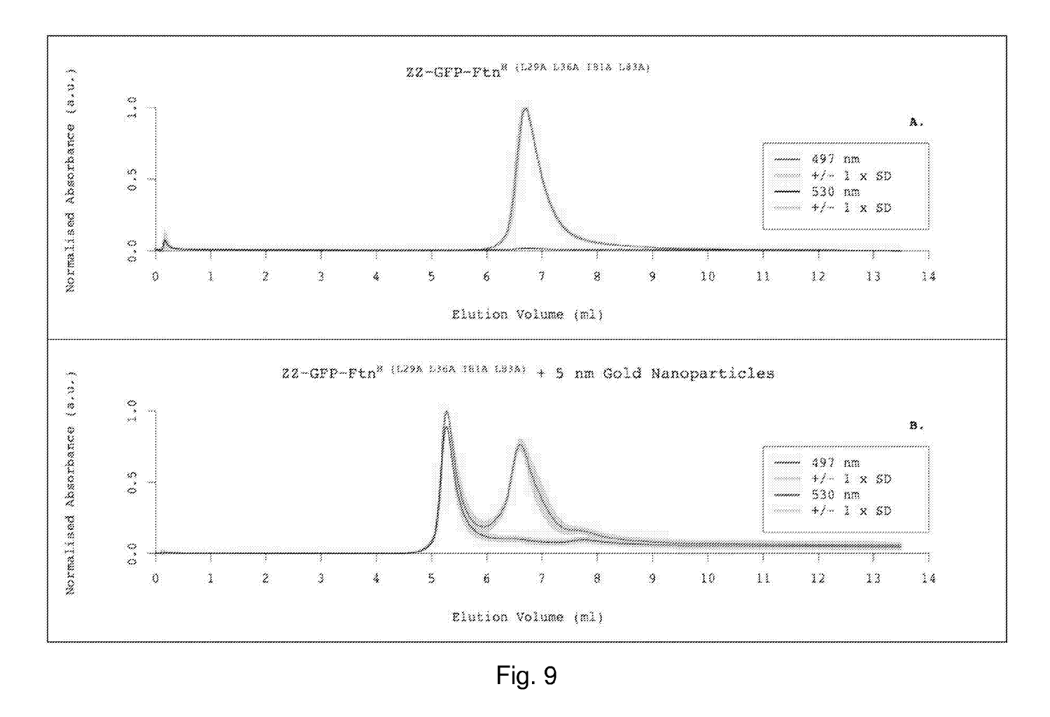

FIG. 9 shows reassembly of mutant hFTN. HPLC SEC chromatograms of (A.) ZZ-GFP-hFTN (L29A L36A I81A L83A), (B.) ZZ-GFP-hFTN (L29A L36A I81A L83A) with AuNP. In all chromatograms, the 24-mer elutes at approximately 5.3 ml and the monomer elutes at approximately 6.8 ml. The wt hFTN is seen to elute primarily as 24-mer (panel A). In the presence of AuNP, the AuNP co-elutes with the FTN 24-mer. The dark green line corresponds to the absorbance readings at 497 nm (GFP absorbance) and the dark blue line absorbance at 530 nm (AuNP absorbance). The shading in both instances corresponds to ±1 standard deviation of the mean absorbance readings. Each dataset is comprised of three biological repeats;

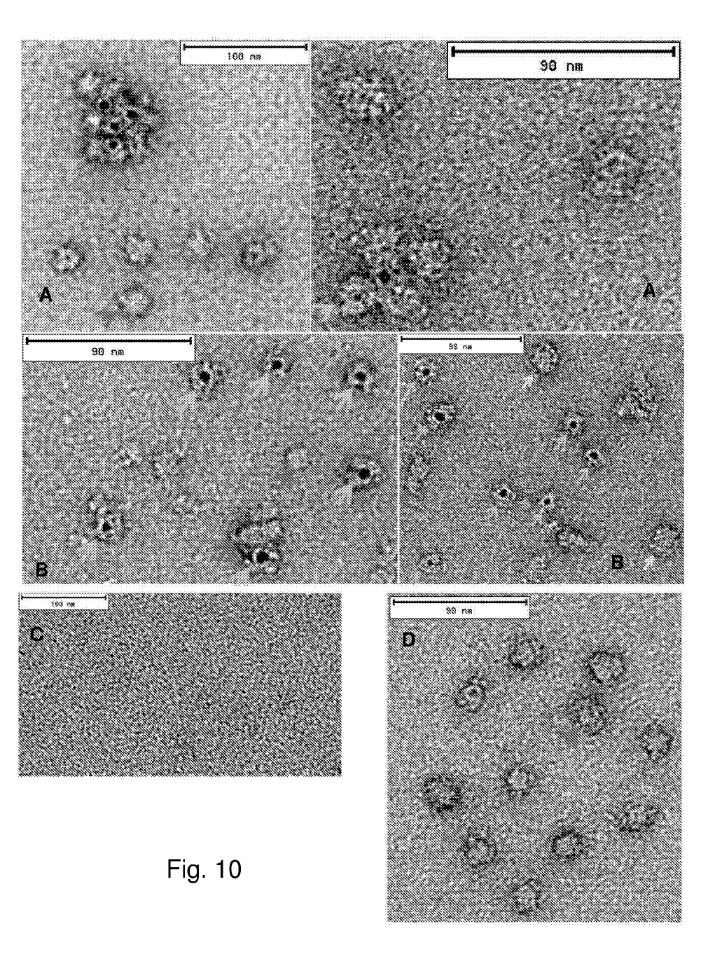

FIG. 10 shows the results of TEM analysis of hFTN with AuNP. TEM analysis of hFTN with AuNP. (A) wt ZZ-GFP-hFTN with AuNP, blue arrows indicate clusters with AuNP, red arrows indicate isolated nanocages; (B) mutant ZZ-GFP-hFTN (L29A L36A I81A L83A) with AuNP, blue arrows indicate nanocages with encapsulated AuNP, red arrows indicate isolated nanocage fragments, yellow arrows indicate empty nanocages; (C) mutant ZZ-GFP-hFTN (L29A L36A I81A L83A) without AuNP (D) wt ZZ-GFP-hFTN without AuNP, red arrows indicate nanocages;

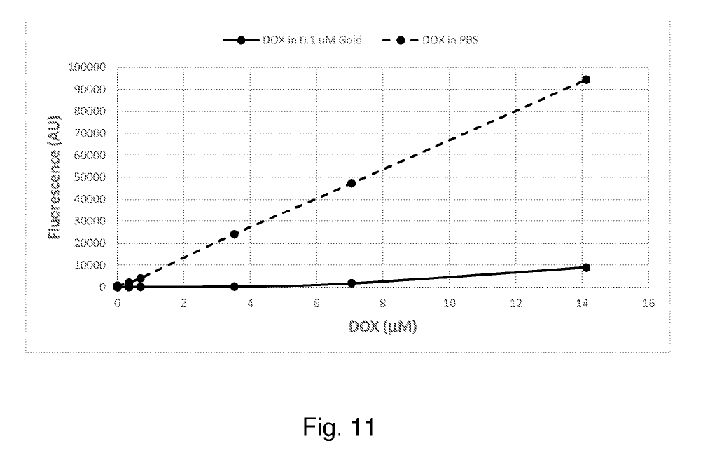

FIG. 11 shows the binding of Doxorubicin to gold nanoparticles. The binding of doxorubicin (Dox) to 5 nm gold nanoparticles was monitored from the fluorescence signal of the Dox. A titration of Dox concentration was measured in PBS either in the presence or absence of 5 nm Au nanoparticles. Fluorescence was measured in a BMG Clariostar plate reader (ex: 482-16; emm: 580-30) and intensity plotted after subtraction of background. Binding of the Dox to the Au causes a significant quenching of the Dox fluorescence;

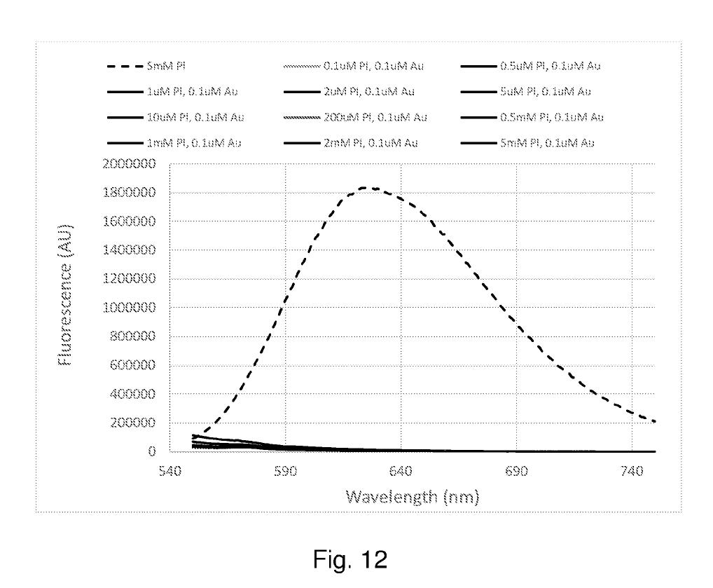

FIG. 12 shows the interaction of propidium iodide with Au nanoparticles. The binding of propidium iodide (PI) to 5 nm gold nanoparticles was monitored from the fluorescence signal of the PI. A titration of PI concentration was measured in PBS either in the presence or absence of 5 nm Au nanoparticles. Fluorescence was measured in a Fluoromax-4 (ex: 493 nm; emm: 550-750) and emission scans are plotted after subtraction of background. Binding of the PI to the Au causes complete ablation of the PI fluorescence;

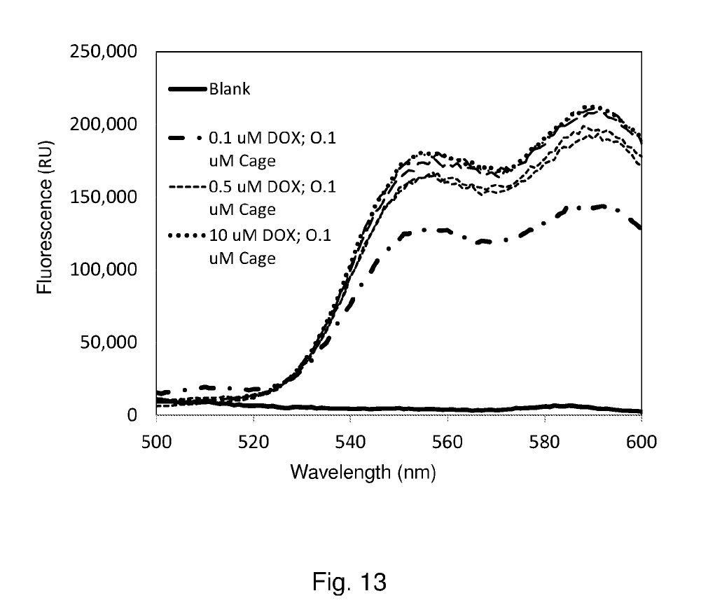

FIG. 13 shows Dox fluorescence in purified nanocage-Au-Dox complexes. Complexes containing hFTN (L29A L36A I81A L83A), Au nanoparticle and Dox were formed by adding the mutant ferritin protein (0.1 μM) to different concentrations of Dox (0.1 μM to 10.0 μM). After 16 h the nanocages formed were purified by HPLC and scanned for Dox fluorescence in a Fluoromax-4 (ex: 482 nm; emm: 500-600);

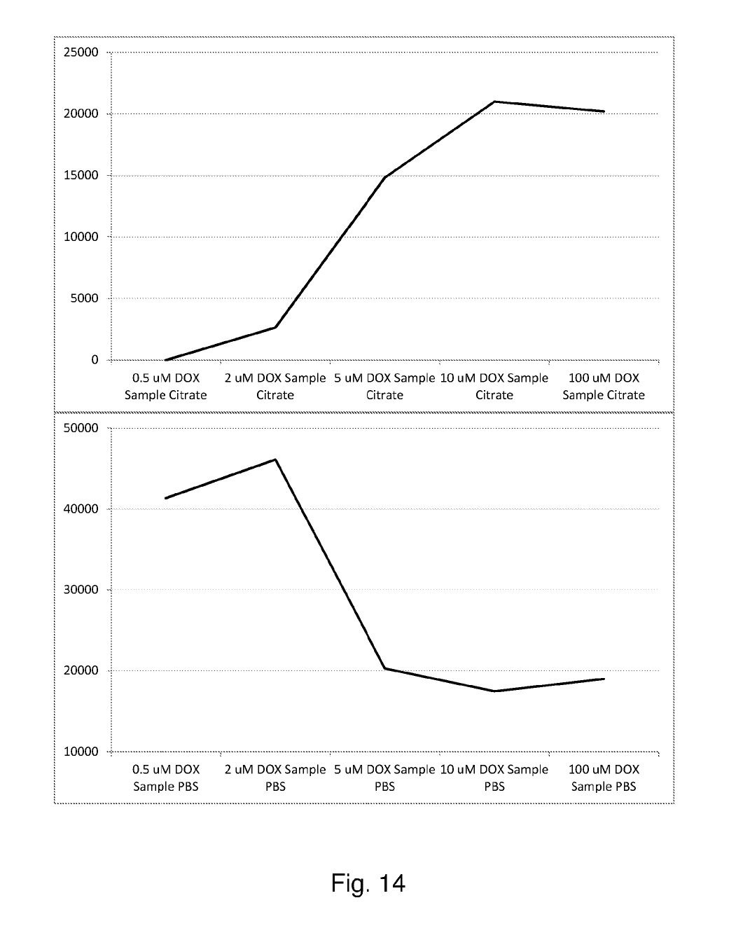

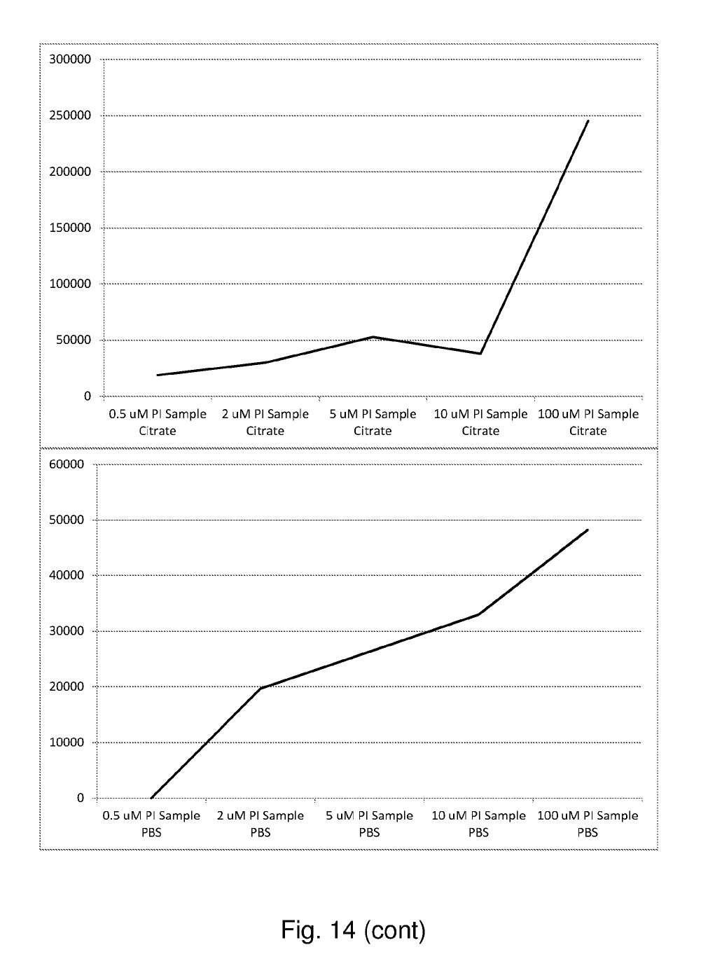

FIG. 14 is mass spectrometry analysis of drug encapsulation. Complexes containing hFTN (L29A L36A I81A L83A), Au nanoparticle and Dox were formed by adding the mutant ferritin protein (0.1 μM) to different concentrations of Dox (0.1 μM to 10.0 μM), Au nanoparticle preparations stabilised with either citrate or PBS (phosphate buffered saline) were used to evaluate if this affected the binding of the drug to the gold. After 16 h the nanocages formed were purified by HPLC and analysed by LC-MS (Agilent 6550), data were quantified using a 20 ppm window for Dox and PI based on a calibrated standard;

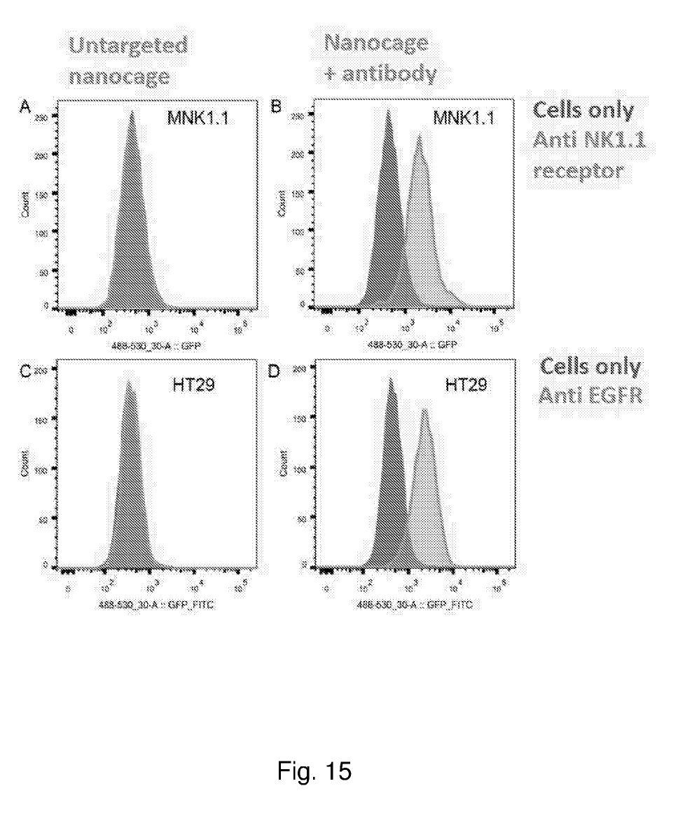

FIG. 15 shows antibody directed cell binding of GFP nanocage. Purified wt ZZ-GFP-hFTN (20 μg) was mixed with either anti-NK1.1 antibody (1 μg) or anti-EGFR antibody (1 μg) in 210 μl of PBS. For each assay, 50 μl of the nanocage-antibody was mixed with 1×10⁶ cells of either HT29 or MNK1.1 in 100 μl. Cells were analysed an a BD Fortessa using the FITC channel (ex 488 nm; emm 530-30 nm) to observe GFP fluorescence. Data show cells only (red histogram, all traces) and those with nanocage alone and no antibody for MNK1.1 cells (A) and HT29 cells (C). Nanocage antibody are shown with MNK1.1 cells (B) and HT29 cells (D);

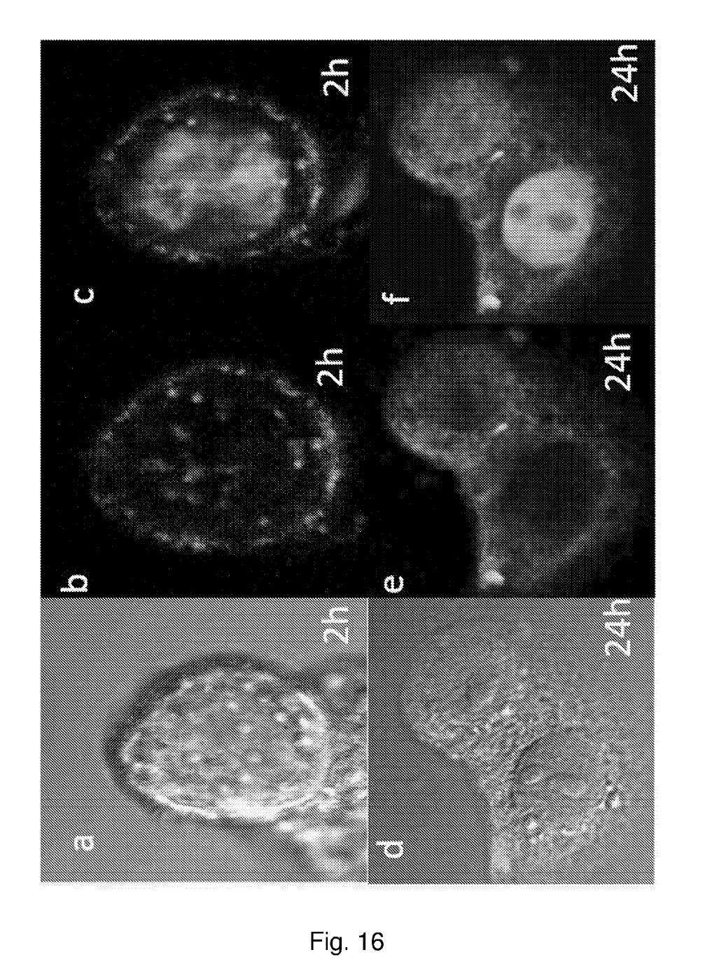

FIG. 16 shows the fate of the antibody targeted nanocage. Confocal microscopy showing a z-slice. Purified Au-ZZ-GFP-hFTN (L29A L36A I81A L83A) was mixed with anti-EGFR antibody (1 μg) in 210 μl of PBS. HT-29 cells were seeded on chamber slides (ibidi) in DMEM medium with 10% FBS overnight for cell attachment. Cells were then treated with the purified nanocage-Au complex (20 μl) at 37° C. for different times (panels a-c, 2 h, d-f, 24 h). After the incubation, the cells were washed with cold PBS, fixed in 4% cold Paraformaldehyde, and permeabilized with 0.1% Triton X-100. To visualize lysosomes, the cells were further incubated with an anti-Lamp1 (1:100; Biolegend) for 1 h after blocking by 1% BSA . The cells were then washed three times with PBS and incubated with Cy₃ Goat anti mouse IgG (1:500; Biolegend) for 1 h. Nuclei were stained with DAPI (1 μg/mL; Sigma) for 2 min at room temperature and then again washed with PBS; cells were covered with mounting media and coverslip and observed under microscope (Brightfield, DAPI ex 405 nm; emm 420-480 nm: CY3 ex 550 nm; emm 560 nm: Dox ex 488 nm; emm 550-590 nm) Zeiss LSM 510 inverted confocal microscope. Images are shown with GFP signal in green, Lmp1 signal in Red and DAPI in blue;

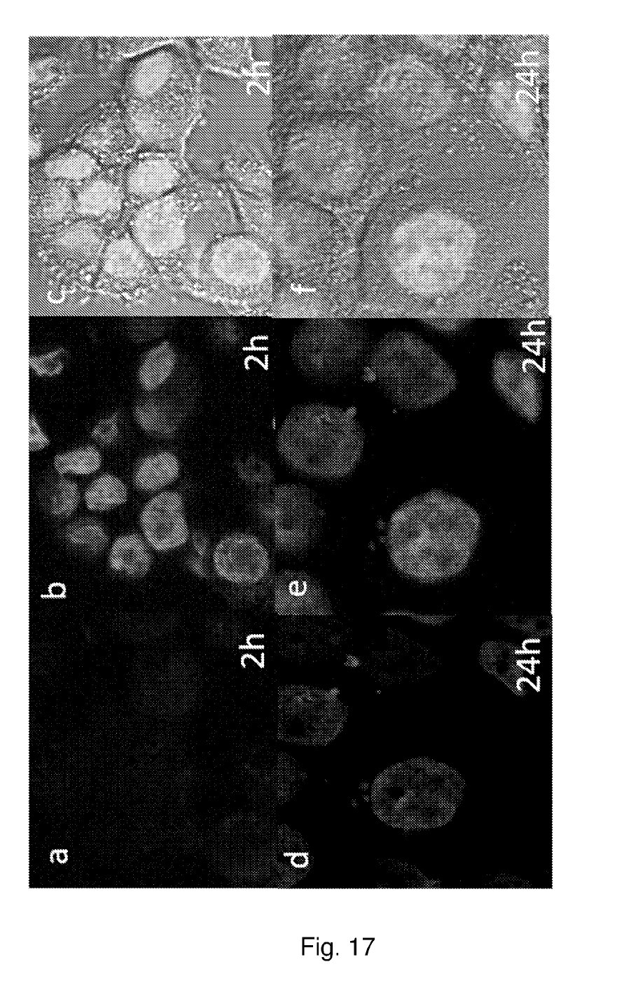

FIG. 17 shows delivery of Dox to cells by encapsulated nanocage. Confocal microscopy showing a z-slice. Purified Dox-Au-ZZ-hFTN (L29A L36A I81A L83A) (100 μl of 30 nM) was mixed with anti-EGFR antibody (1 μg) in 210 μl of PBS. HT-29 cells were seeded on chamber slides (ibidi) in DMEM medium with 10% FBS overnight for cell attachment. Cells were then treated with the nanocage-antibody complex (100 μl) at 37° C. for different times (panels a-c, 2 h, d-f, 24 h). After the incubation, the cells were washed with cold PBS, fixed in 4% cold Paraformaldehyde, and permeabilized with 0.1% Triton X-100. Nuclei were stained with DAPI (1 μg/mL; Sigma) for 2 min at room temperature and then again washed with PBS; cells were covered with mounting media and coverslip and observed under microscope (Brightfield: DAPI ex 405 nm; emm 420-480 nm: Dox ex 488 nm; emm 550-590 nm) Zeiss LSM 510 inverted confocal microscope. Images are shown with Dox signal in red, and DAPI in blue;

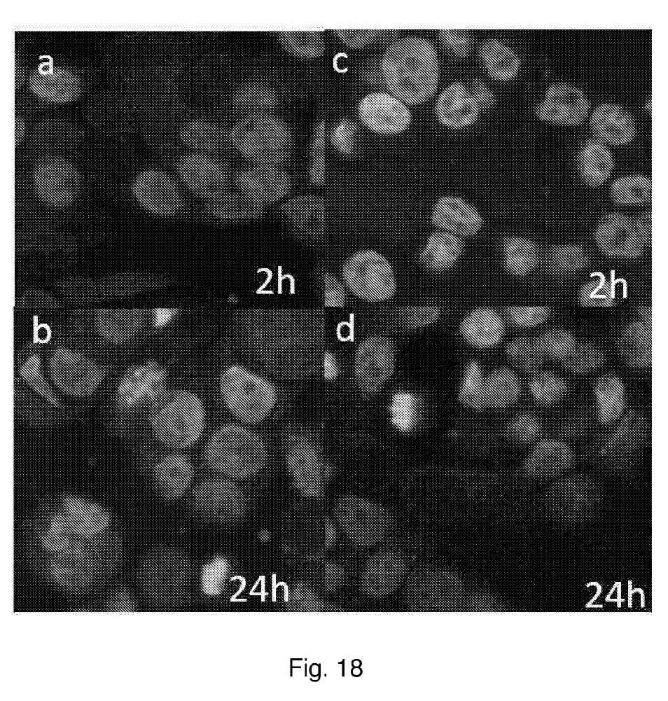

FIG. 18 shows delivery of PI to cells by encapsulated nanocage. Confocal microscopy showing a z-slice. Purified Dox-Au-ZZ-hFTN (L29A L36A I81A L83A) (100 μl of 30 nM) was mixed with anti-EGFR antibody (1 μg) in 210 μl of PBS. HT-29 cells were seeded on chamber slides (ibidi) in DMEM medium with 10% FBS overnight for cell attachment. Cells were then treated with the nanocage-antibody complex (100 μl) at 37° C. for different times (panels a-c, 2 h, d-f, 24 h). After the incubation, the cells were washed with cold PBS, fixed in 4% cold Paraformaldehyde, and permeabilized with 0.1% Triton X-100. Nuclei were stained with DAPI (1 μg/mL; Sigma) for 2 min at room temperature and then again washed with PBS; cells were covered with mounting media 480 nm: PI ex 535 nm; emm 617 nm) Zeiss LSM 510 inverted confocal microscope. Images are shown with Dox signal in red, and DAPI in blue;

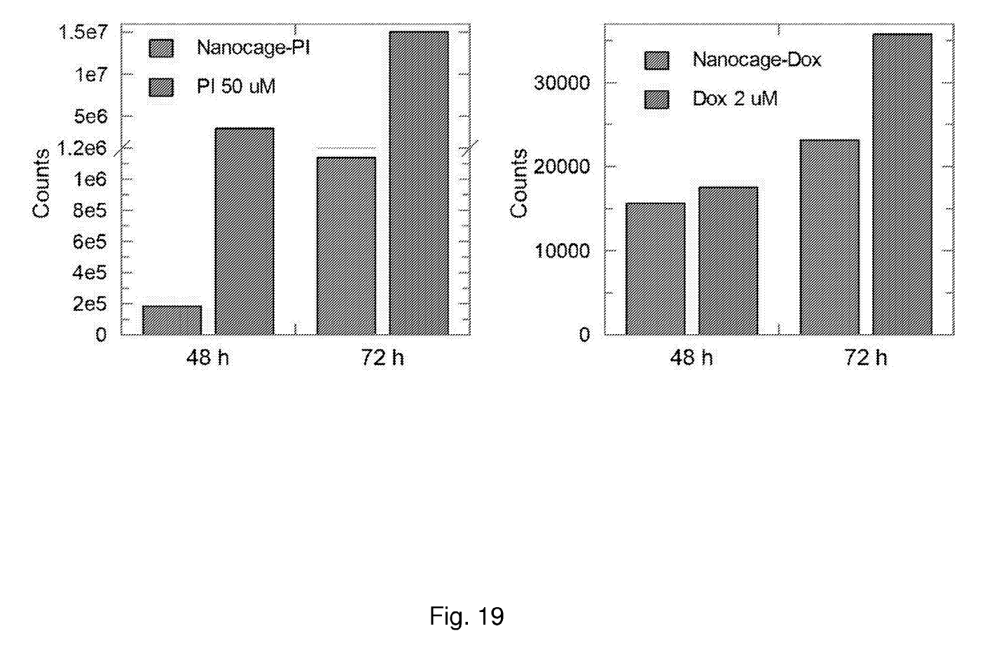

FIG. 19 shows purified Dox/PI-Au-ZZ-hFTN (L29A L36A I81A L83A) (100 μl of ˜30 nM) was mixed with anti-EGFR antibody (1 μg) in 210 μl of PBS. HT-29 cells were grown in DMEM medium with 10% FBS overnight. Cells were then treated with the nanocage-antibody complex (100 μl) at 37° C. for different 48 h and 72 h. After incubation, the cells were washed 3× with cold PBS. Re-suspended cells were analysed by LC-MS (Agilent 6550), data were quantified using a 20 ppm window for Dox and PI based on a calibrated standard;

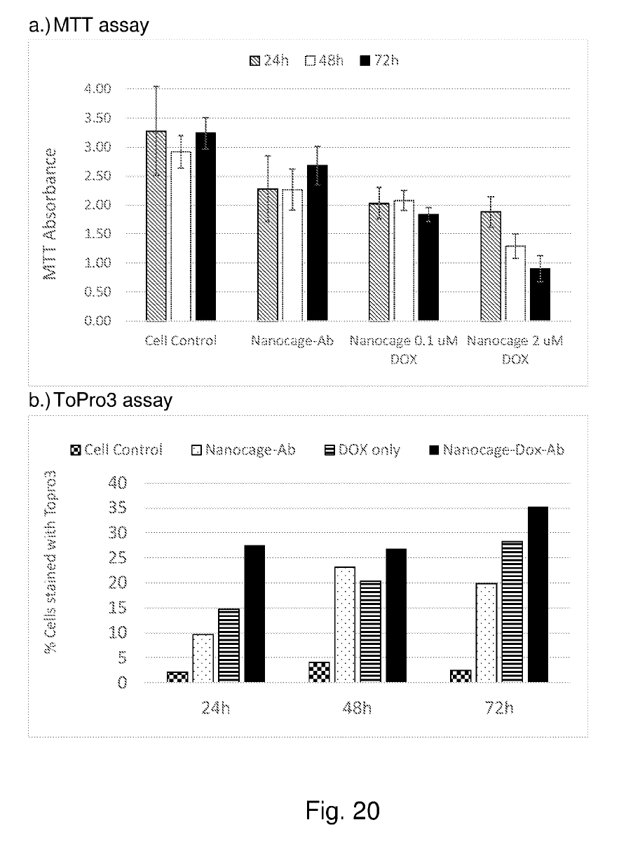

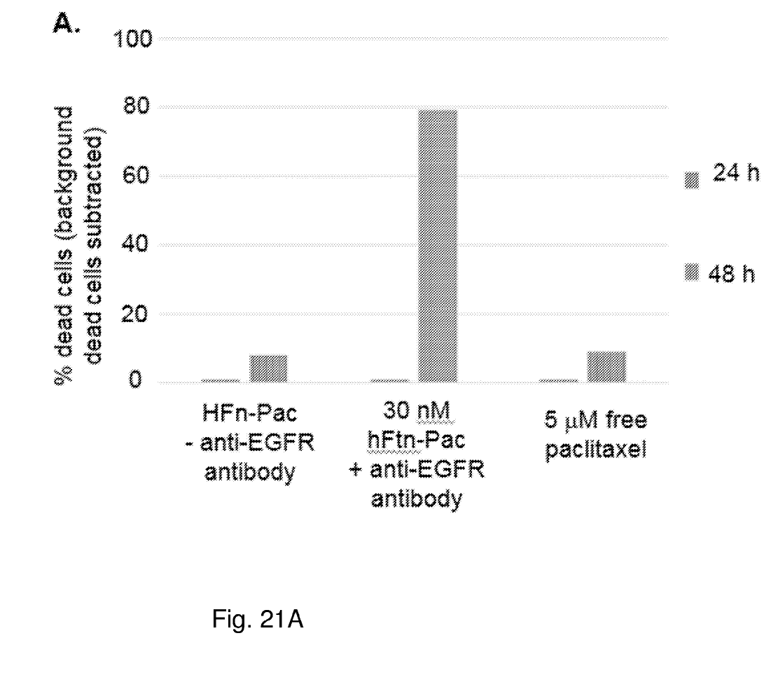

FIG. 20 shows phenotypic assays of drug delivery. a) MTT assay. Purified Dox-Au-ZZ-hFTN (L29A L36A I81A L83A) (100 μl of 30 nM), prepared by loading with either 0.1 μM or 2.0 μM DOX, was mixed with anti-EGFR antibody (1 μg) in 210 μl of PBS. Cells were cultured on a three 96 well plate (5000 cells/well) Then, cells were incubated with the prepared nanocage-antibody complexes. At the indicated time points (24, 48, 72 hours), cells were washed with PBS and then incubated for 3 h at 37° C. with 3-(4,5-dimethyl-2-thiazolyl)-2,5-diphenyl-2H-tetrazolium bromide (MTT) stock (5 mg/mL) diluted in PBS ( 1/10th of culture volume typically 20 μL). After incubation, MTT solubilizing solution (1:1 of DMSO and isopropyl alcohol) was added to each well to solubilise the MTT formazan crystals Absorbance was read after shaking for 10 minutes at 37 C in a BMG Clariostar at 590 nm and b) ToPro3 assay: cells and nanocages were prepared as above (using 2.0 μM DOX). Prior to assay, cells were mixed with ToPro-3 staining solution (1 μM) and incubated for 30 min, washed with PBS and analysed on a BD Fortessa (640 nm ex; 670/14 emission), data was analysed using FlowJo;

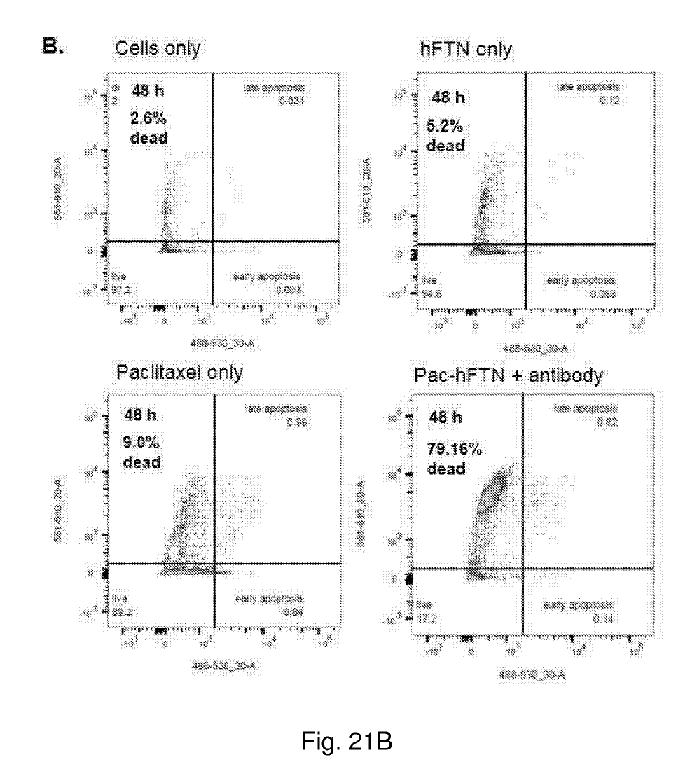

FIG. 21 shows a phenotypic cell killing assay using Vybrant staining and flow cytometry for nanocage delivered Paclitaxel (Pac). Purified Pac-Au-ZZ-hFTN (L29A L36A I81A L83A) (100 μl of 30 nM), was prepared by loading with 5.0 μM Pac and unincorporated drug removed by Zorbax spin column; this was mixed with anti-EGFR antibody (1 μg) in PBS and exposed to 5×10⁵ live cells. A shows the degree of dead cells observed after 24 h and 48 h for the drug loaded nanocage in the absence of antibody, in the presence of antibody and for 5 μM free drug. B shows the flow cytometry dot plots for cells only, cells with hFtn only, free drug only and Pac loaded nanocage with antibody; the upper left quadrant shows dead cells and the lower left live cells;

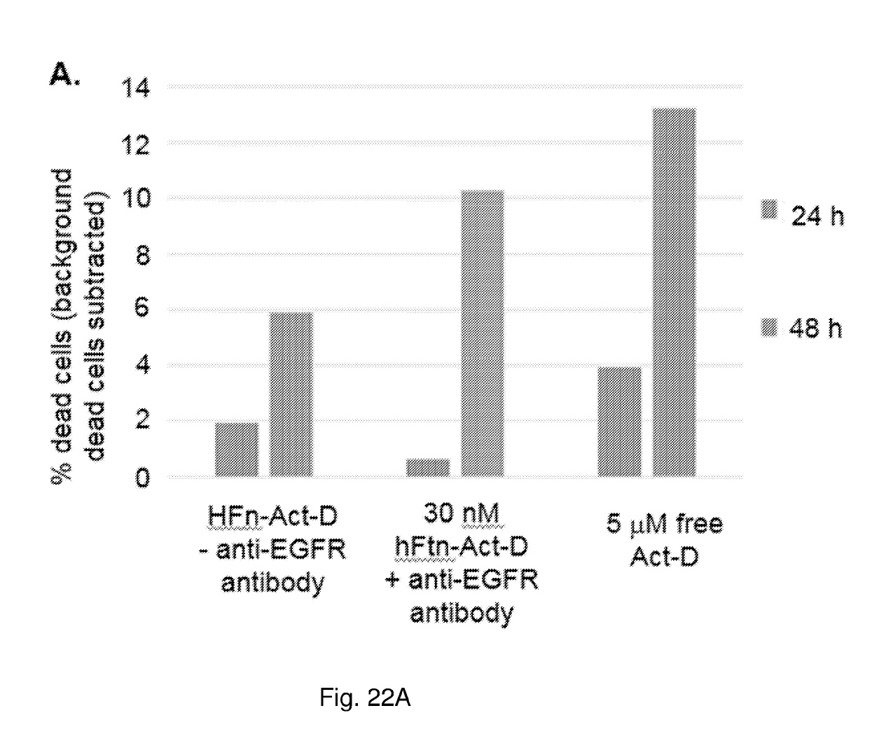

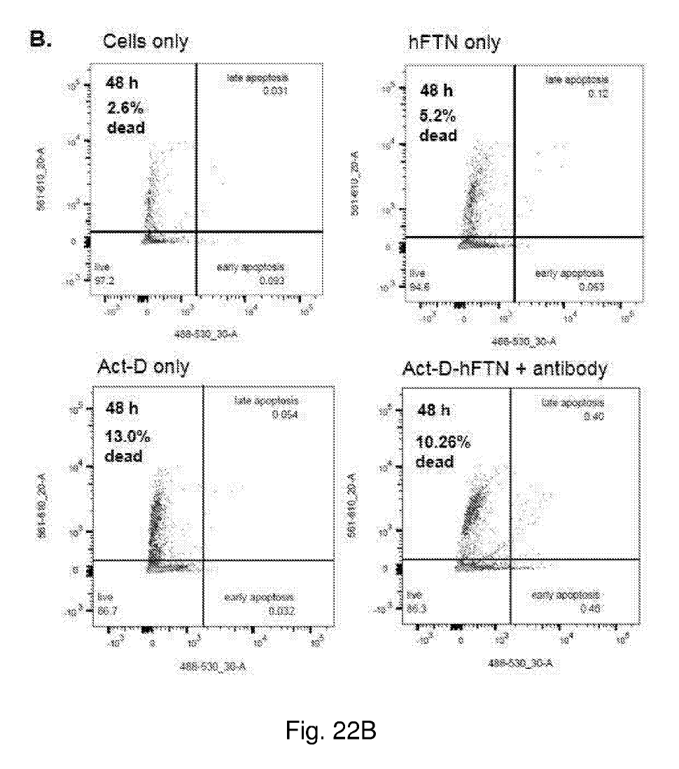

FIG. 22 shows a phenotypic cell killing assay using Vybrant staining and flow cytometry for nanocage delivered Actinomycin-D (Act-D). Purified Act-D-Au-ZZ-hFTN (L29A L36A I81A L83A) (100 μl of 30 nM), was prepared by loading with 5.0 μM Act-D and unincorporated drug removed by Zorbax spin column; this was mixed with anti-EGFR antibody (1 μg) in PBS and exposed to 5×10⁵ live cells. A shows the degree of dead cells observed after 24 h and 48 h for the drug loaded nanocage in the absence of antibody, in the presence of antibody and for 5 μM free drug. B shows the flow cytometry dot plots for cells only, cells with hFtn only, free drug only and Act-D loaded nanocage with antibody; the upper left quadrant shows dead cells and the lower left live cells; and

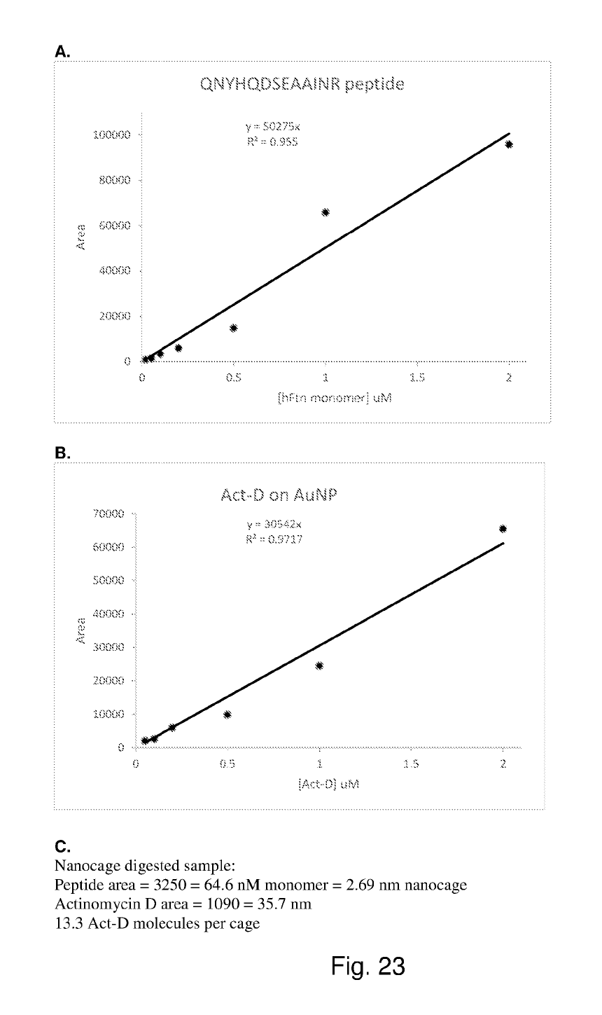

FIG. 23 shows mass spectrometry results performed to determine quantitation of Act-D as encapsulated within the hFtn nanocage. (A) A calibration curve was performed for the monomer His-ZZ-hFTN(L29A L36A I81A L83A) based on the QNYHQDSEAAINR peptide. (B) A calibration curve was performed for Actinomycin-D bound to Au nanoparticles. (C) HPLC purified Act-D encapsulated nanocage Act-D-His-ZZ-Au-hFTN (L29A L36A I81A L83A) was then analysed by the same method on the same day. Areas for the peptide and Act-D were determined and based on the calibration curves in A and B there were calculated to be 13.3 Act-D molecules per cage.

### EXAMPLES

It has previously been demonstrated that the thermostable ferritin from Archaeoglobus fulgidus (A. fu) is stable in a dimeric form at low salt and reversibly forms nanocage structures on transition to high salt^{15, 16}. However, while in the destabilised dimeric state, it could interact with a gold nanoparticle to form a ferritin-encapsulated gold nanoparticle. Other efforts to encapsulate either drugs or metal cores into ferritin rely on the fact that it dissociates into its constituent dimers at low pH and can reform the nanocage on transition back to neutral pH^{3, 17, 18}. However, this pH change is also partially destructive and it impacts the integrity of the reformed nanocage¹⁸. The concept of an ordered disassembly and reassembly under mild conditions that does not damage the ferritin is therefore an attractive option for the creation of nanocages based on ferritin. So far this has not been achieved with anything other than A. fu ferritin. The inventors, therefore, decided to create nanocages that exhibit ordered disassembly and reassembly without the use of harsh denaturation conditions.

Materials and Methods

Protein Expression and Purification

A plasmid encoding the recombinant protein of interest was transformed into E. coli BL21-DE3. Single colonies were suspended in 8×5 mL LB media containing chloramphenicol (34 μg ml⁻¹)and grown overnight at 37° C. and 220 rpm in a shaker incubator. Starter culture inoculated at 1:100 dilution for 2 hours at 37° C. 220 rpm, ˜10 mL starter culture in 500 mL LB media containing chloramphenicol (34 μg ml⁻¹), using two 2-litre conical flasks. Once an OD600 of reached 0.4-0.5 culture induced with 1 mM IPTG, and protein expressed for ˜6 hours until OD600 reaches 1.7-2.2. Initially culture harvested into 2×500 mL centrifuge tubes (5000 rpm, 4° C., 10 mins) pellets were stored at −80° C.

Pellet cells were thawed on ice in lysis buffer (1×PBS, 50 mM imidazole, 100 mM NaCl, pH 7.2) containing 1 protease inhibitor cocktail tablet (Roche). Resuspended cells were sonicated for 2×10 mins (amplitude 40%, pulse 2 seconds on 2 seconds off) and then centrifuged (15000 rpm, 4° C., 40 min.). Initial purification conducted with immobilized metal ion affinity chromatography (His-tag), His-tag beads (chelating sepharose fast flow, GE healthcare) charged with NiCl₂ were added to the supernatant on ice and mixed every 10 mins for 1 hour. This mix was made up to 50 mL using lysis buffer and centrifuged (3000 rpm, 4° C., 2 mins). This was repeated 2-3 times with lysis buffer until the discarded supernatant was clear. Beads are loaded onto column, washed twice with 10 mL lysis buffer and eluted with 10 mL elution buffer (1× PBS, 300 mM imidazole, 100 mM NaCl, pH 7.4). Eluted protein was dialysed overnight (100 mM NaCl, 1×PBS, pH 7.2). Protein was concentrated to 1-2 mL using Amicon ultra-15 centrifugal filter unit (3000 rpm, 4° C., ˜30 mins). Further purification was conducted using size exclusion chromatography. GE Akta FPLC system combined using a Superdex 200 gel filtration column at a 0.5 mL/min flow rate (buffer 50 mM TRIS, 200 mM NaCl, pH 7.5). Fractions containing protein were combined and concentrated to 1-2 mL (3000 rpm, 4° C., ˜1 hour). When used for storage mixed equally (by volume) with 80% glycerol.

HPLC Size Exclusion Chromatography (SEC)

Once purified, the quaternary structures of our protein samples were analysed using size exclusion chromatography (SEC) on a high performance liquid chromatography (HPLC) platform (Thermo Surveyor with diode array detector). SEC was conducted on a refrigerated (10° c.) TSK-GEL G3000SWXL column (Tosoh Bioscience LLC, Montgomeryville, Pa.) equilibrated with filtered (0.22 μm filter) Buffer A (100 mM NaCl, 50 mM HEPES, pH 7.2). Prior to sample injection, protein samples were dialysed overnight against Buffer A, which was also used as the mobile phase in the SEC experiments. For each experiment, 0.2 mg of protein was loaded onto the column. SEC experiments were run for 45 minutes at a flow rate of 0.3 ml/min. A diode array was used to measure the absorbance properties of protein sample as it eluted from the column. Specifically, we combined high frequency (10 Hz) monitoring at three wavelengths (λ=280 nm, 497 nm, 530 nm) with periodic wavescans (230-700 nm). The column was calibrated using a series of standard commercial proteins, which enabled us to subsequently estimate the molecular masses of our samples. The concentration of protein samples was calculated using absorbance spectroscopy with an extinction coefficient of 15,930 cm⁻¹ M⁻¹ at 280 nm for human light chain ferritin and 18,910 cm⁻¹ M⁻¹ at 280 nm for human heavy chain ferritin. Extinction coefficients for other fusion proteins, extinction coefficients were calculated using the ExPASy ProtParam tool. The ratio of Bfr subunits to heme molecules was calculated using an extinction coefficient for heme of 137,000 cm⁻¹ M⁻¹ at 417 nm.

Nanocage Fabrication and Drug Encapsulation

The purified ferritin protein was mixed with 5 nm gold nanoparticles (Sigma Aldrich). Stoichiometry was estimated from protein concentration and stated number of gold particles per unit volume, calculated to give 24 protein monomers per gold nanoparticle. Where drugs were encapsulated, these were added to the Au nanoparticles at the concentration indicated at room temperature, ferritin was added between 1 min and 30 min after. Gold nanoparticles and protein were co-incubated for 12 hours at 4° C. If needed concentrated to 1-2 mL (3000 rpm, 4° C.) and then purified using HPLC size exclusion chromatography, as above. Fractions containing nanocage were combined and concentrated to 1-2 mL (3000 rpm, 4° C., ˜1 hour). When used for storage mixed equally (by volume) with 80% glycerol.

Concentrations of gold nanoparticle encapsulated ferritin nanocages were calculated based on the sum of the extinction coefficient at 280 nm of 5 nm gold nanoparticles (1.66×10⁷ M⁻cm⁻¹) and the extinction coefficient at 280 nm for the relevant protein components also present.

Transmission Electron Microscopy Analysis

Protein samples were mounted on carbon coated copper grids. The grids were prepared in advance using glow discharge. This technique increases the hydrophilicy of the grid allowing the protein sample to adhere to the carbon coating. After the protein sample had been loaded onto the grid, a negative stain was applied (uranyl acetate) to provide contrast.

Fluorescence Analysis

Fluorescence measurements were performed either on a Jobin Yvon Fluoromax 4 with a 400 μl cuvette using excitation and emission wavelengths as stated and slit widths of 5 nm. Alternatively a BMG Clariostar plate reader was used with filters or monochromator settings as described using clear bottom black wall plates. (Greiner Bio-One).

LCMS

Purified protein and cell extract samples were analysed by LCMS on an Agilent 6550. LC separation was achieved using a 1290 Infinity system (Agilent, Santa Clara, Calif.) and a Vydac 214MS C4 column, 2.1×150 mm and sum particle size, (Grace, Columbia, Md.) at a temperature of 35° C. with a buffer flow rate of 0.2 ml/min. with a denaturing mobile phase: buffer A was 0.1% formic acid in water and buffer B was 0.1% formic acid in acetonitrile. Elution of components was achieved using a linear gradient from 3% to 40% buffer B over 18.5 min. On-line mass spectra were accumulated on a 6550 quadrupole time-of-flight instrument with a dual electrospray Jet Stream source (Agilent). Mass spectra were acquired of the m/z range of 100-1700 at a rate of 0.6 spectra per second. Targeted MS/MS were acquired over the range of 100-1700 Da with a 1.3 Da precursor isolation window and a collision energy of 15 eV.

Proteolytic Digestion of Human Ferritin Mutant Monomer and Nanocage

The protocol was adapted from the manufacturer's instructions. Nanocage was incubated in 8M Urea in 50 mM Tris-HCl (pH 8) with 4 mM DTT and heated at 95° C. for 20 minutes. After denaturation the reaction mixture was cooled and 50 mM NH₄HCO₃ was added such that the urea concentration is below 1M. Modified Trypsin was then added to a final protease:protein ratio of 1:100 and incubated overnight at 37° C. for complete digestion. Human Ferritin mutant monomer samples did not require urea denaturation and were only digested with Trypsin.

Standard solutions of varying concentrations (0, 0.05, 0.1, 0.2, 0.5, 1, 2 μM) were prepared for the drug in buffer, drug on gold nanoparticles and human Ferritin mutant monomer.

Targeted LC-MS/MS Measurements

The targeted LC-MS/MS method was applied using an Agilent 1290 LC system coupled to an Agilent 6550 quadrupole-time-of-flight (Q-ToF) mass spectrometer with electrospray ionization (Agilent, Santa Clara, Calif.). The LC column used was an Agilent Zorbax Extend C-18, 2.1×50 MM and 1.8 um particle size. The LC buffers were 0.1% formic acid in water and 0.1% formic acid in acetonitrile (v/v). In addition to the target molecule, two diagnostic tryptic peptides for the protein to be measured were selected for the targeted LC-MS/MS method. This was achieved by comparison of the peptides identified from the protein by auto-MS/MS analysis of digested samples with those predicted to be suitable for measurement by LC-MS/MS using Peptide Selector software (Agilent, Santa Clara, Calif.). By combining the recorded LC retention times and target precursor masses, a method to determine the concentration of both the target molecule and protein was developed.

Quantitation was based on the LC retention times of standards and the area of accurately measured diagnostic precursor or fragment ions. The protonated molecules of each peptide, [M+2H]²⁺, were targeted and subjected to collision induced dissociation, with product ions accumulated throughout the targeted period. Concentrations were calculated using the integrated area of the peak corresponding to the elution of the molecule or peptide of interest at the retention time of the standards. This was measured from either the response for the precursor ion or for a fragment ion from the product ion spectrum of each entity. Calibration curves generated from the standards were used to calculate concentrations.

Flow Cytometry

Flow cytometry was performed on a BD Fortessa using the FITC channel to observe GFP (ex 488 nm; emm 530-30 nm; ToPro-3 was imaged in red channel (640 nm ex; 670/14 emission). Data was analysed using Flow-Jo software.

Cell Preparation for LCMS Analysis

Cells were lysed using a bead beading process. Cells were pelleted at 7 k rcf for 10 min. and dissolved in 100 μl methanol and vortexed until homogenous. 50 μl of acid washed glass beads (Sigma) were added. Cells were then vortexed for 30 s and kept on ice for 30 s four times before centrifugation at 14 krpm at 4° C. for 15 min. Supernatant was then taken for LCMS analysis as above.

Immunofluorescence

Cells were washed twice with PBS and fixed with 4% formaldehyde for 10 minutes and then washed 3× with PBS. Cells were then permeabilised with 0.1% TX-boo/PBS for 15-20 minutes and wash 3×. Cells were then blocked with 5% normal goat serum/PBS or 1% BSA/PBS for 45 minutes (no washing required). The primary antibody was diluted in blocking solution and applied for 2 h (or overnight at 4° C.). Wash 4× thoroughly to remove unbound primary antibody. Cells were then incubatee with the secondary antibody for 1 h, diluted in blocking solution or wash buffer. The secondary antibody was then aspirated and, if required, incubated with DAPI [1 μg/mL] in PBS for 10 minutes and washed 4×. Coverslip was then dipped into H₂O to remove residual salts of the wash buffer. A drop of mounting medium was added and the slide sealed. Antibodies used were as stated in Figure legends.

### MTT Assay

Purified Dox-Au-ZZ-hFTN (L29A L36A I81A L83A) (100 μl of 31 nM) was prepared by loading with either 0.1 μM or 2.0 μM DOX, was mixed with anti-EGFR antibody (1 μg) in 210 μl of PBS. Cells were cultured on a three 96 well plate (5000 cells/well) Then, cells were incubated with nanocage constructs to be tested. At the indicated time points (24, 48, 72 hours), cells were washed with PBS and then incubated for 3 h at 37° C. with 3-(4,5-dimethyl-2-thiazolyl)-2,5-diphenyl-2H-tetrazolium bromide (MTT) stock (5 mg/mL) diluted in PBS ( 1/10th of culture Volume typically 20 uL). After incubation, MTT solubilizing solution (1:1 of DMSO and isopropyl alcohol) was added to each well to solubilise the MTT formazan crystals Absorbance was read after shaking for 10 minutes at 37° C. plate shaker at testing wavelength of 590 nm.

ToPro-3 Assay

Purified Dox-Au-ZZ-hFTN (L29A L36A I81A L83A) (100 μl of 31 nM) was prepared by loading with 2.0 μM DOX, was mixed with anti-EGFR antibody (1 μg) in 210 μl of PBS. Cells were cultured on a three 96 well plate (5000 cells/well) Then, cells were incubated with nanocage constructs to be tested. Prior to assay, cells were mixed with ToPro-3 staining solution (1 μM) and incubated for 30 min, washed with PBS and analysed on a BD Fortessa (640 nm ex; 670/14 emission), data was analysed using FlowJo.

Phenotypic Cell Death Assays Using Vybrant Cell Staining Assay

HT-29 cells were trypsinised and cell viability was assessed using the Trypan blue exclusion assay—count cells treated with Trypan blue dye using a haemocytometer, and determine the volume of cell suspension that contains 5×10⁵ live cells. Live cells (5×10⁵) seeded into a 12-well plate with a final volume of 500 μL. This final 500 μL volume will consist of 5×10⁵ cells+medium+drug-Au-ZZ-hFTN+anti-EGFR antibody (0.5-1 μg). The plate was incubated in a tissue culture incubator set at 37° C., 5% CO₂, 95% humidity for 24 h or 48 h before the experiment was stopped. Uptake of drug-Au-ZZ-hFTN by cells was stopped by removing the 500 μL solution containing test or control compounds, trypsinising cells and pelleting cells in preparation for cytotoxicity assays, e.g. Vybrant cell apoptosis assay. Prior to staining the cells were centrifuged (3000 rpm for 2 min) and then washed in cold PBS before a second centrifugation step at 3000 rpm for 2 min in a microcentrifuge. The supernatant was removed and discarded before the cell pellet was resuspended in 1 mL ice cold sterile 1×PBS containing YOPRO and PI stain. The staining solution was prepared by adding 0.25 μL YOPRO (Component A) and 0.25 μL PI (Component B) stock solutions to 1 mL PBS. Volumes were scaled up for the number of samples that require staining, e.g. 10 samples=2.5 μL of each stain in 10 mL PBS. The cells were incubated in staining solution on ice for 20 min. Within 30 min after the incubation period, the stained cells were analysed by flow cytometry, using 488 nm excitation with green fluorescence emission for YOPRO R-1 (i.e., 530/30 bandpass) and red fluorescence emission for propidium iodide (i.e., 610/20 bandpass), gating on cells to exclude debris. Single-color stained cells were used to perform standard compensation. The stained cell population will separate into three groups: live cells show a low level of green fluorescence, apoptotic cells show an incrementally higher level of green fluorescence, and dead cells show both red and green fluorescence.

Microscopy

The cellular uptake and distribution of HFn nanocage were studied by confocal microscope (Zeiss LSM 510). Briefly, HT-29 cells were seeded on chamber slides (ibidi) in DMEM medium with 10% FBS overnight for cell attachment. Cells were then treated with HFn at 37° C. for different times. After the incubation, the cells were washed with cold PBS, fixed in 4% cold Paraformaldehyde, and permeabilized with 0.1% Triton X-100. To visualize lysosomes, the cells were further incubated with an anti-Lamp1 (1:100; Biolegend) for 1 h after blocking by 1% BSA. The cells were then washed three times with PBS and incubated with Cy₃ Goat anti mouse IgG (1:500; Biolegend) for 1 h. Nuclei were stained with DAPI (1 μg/mL; Sigma) for 2 min at room temperature and then again washed with PBS; cells were covered with mounting media and coverslip and observed under microscope (Brightfield, DAPI ex 405 nm; emm 420-480 nm: CY3 ex 550 nm; emm 560 nm: PI ex 435 nm; emm 617 nm).

Ferritin

The inventors have used ferritin from different biological sources: bacterioferritin (Bfr) was isolated from E. coli and contains 24 subunits and 12 heme groups that bind between the dimeric protein interface. Human ferritin (FTN) can be composed of the light chain ferritin subunit (lFTN) or heavy chain ferritin subunit (hFTN), or a combination of both. By expressing either lFTN or hFTN in E. coli it is possible to create ferritin nanocages that consist of only a single protein monomer.

TEM Method

Protein samples were mounted on carbon coated copper grids. The grids were prepared in advance using glow discharge. This technique increases the hydrophilicity of the grid allowing the protein sample to adhere to the carbon coating. After the protein sample had been loaded onto the grid, a negative stain was applied (uranyl acetate) to provide contrast. After staining, the samples were imaged using transmission electron microscopy (TEM).

### Example 1

### Bacterioferritin

To assess the formation of protein nanocages with E. coli bacterioferritin (Bfr), the bfr gene was amplified from the E. coli genome and cloned into an expressing construct. Two variants of the gene were generated, one (SEQ ID No. 5) included an N-terminal His tag for purification, and the second (SEQ ID No. 9) contained a C-terminal gold binding peptide (AuBP). Metal binding peptides have been shown to provide a mechanism for coordinating the binding of proteins to metallic surfaces¹⁹ and it had been shown that the addition of the Au binding peptide could facilitate the encapsulation of a gold nanoparticle within the ferritin cavity¹⁵.

Surprisingly, the addition of the N-terminal His-tag meant that the Bfr did not purify in its nanocage composition, but as individual monomers (see FIG. 1). After addition of a 5 nm gold nanoparticle (AuNP) and incubation overnight, the protein containing the AuBP had formed a higher order structure consistent with a nanocage being formed around the Au nanoparticle (see FIG. 2). Transmission electron microscopy (TEM) of the purified nanocage complex demonstrated that the nanocage had indeed formed around the AuNP (see FIG. 3).

The very subtle modification of the Bfr sequence with an N-terminal purification tag appears to have been sufficient to destabilise the nanocage structure of Bfr under normal conditions. The use of a C-terminal AuBP is sufficient to establish AuBP templated assembly of a nanocage without using harsh denaturation conditions.

### Example 2

### Human Ferritin Subunit Engineering

Expression and purification of the human heavy and light chain ferritins (hFTN; lFTN) from E. coli produced stable nanocage structures. The addition of an N-terminal His purification tag to either hFTN or lFTN did not destabilise the higher order cage structure. The inventors therefore sought to destabilise the cage structure based on engineering of the protein amino acid sequence. In forming the higher order 24-mer nanocage structure, the ferritin subunits first assemble into dimers via the symmetrical dimer interface (see FIG. 4). Using considerable inventive endeavour, the inventors conducted detailed structural analysis of the dimers, and demonstrated that this is the most stable interface in the nanocage and so would provide a good basis from which to destabilise the tertiary structure.

147 structures of conserved ferritin proteins were analysed to identify evolutionarily conserved hydrophobic residues at the dimer interface of human ferritin proteins that contain at least one hydrophobic residue (see Table 1).

TABLE 1
Conserved domains at the dimer interface containing
at least one hydrophobic residue

| lFTN | hFTN | lFTN & hFTN |
|---|---|---|
| RLLKM (SEQ ID No: 23) | GRIFL (SEQ ID No: 19) | QDIKK |
| | | (SEQ ID No: |
| | | 29) |
| LYLQA (SEQ ID No: 24) | LELYA (SEQ ID No: 20) | |
| TYLSL (SEQ ID No: 25) | VYLSM (SEQ ID No: 21) | |
| ALFQD (SEQ ID No: 26) | IFLQD (SEQ ID No: 22) | |
| LGFYF (SEQ ID No: 27) | | |
| DEWGK (SEQ ID No: 28) | | |

Hydrophobic residues within these conserved motifs were then carefully selected for site specific mutagenesis (see FIGS. 4C and 4D). Four mutations were created in the heavy [hFTN (L29A L36A I81A L83A)] and light [lFTN (L32A F36A L67A F79A)] chain variants of FTN according to the conserved motifs identified. These were constructed as N-terminal fusions with GFP (green fluorescent protein) to enable visualisation of the nanocage and either with or without a C-terminal AuBP (SEQ ID No. 7).

For each heavy and light chain variant of FTN, four protein variants were expressed and purified:
(i) wild type FTN with N-terminal GFP;
(ii) wild type FTN with N-terminal GFP and C-terminal AuBP;
(iii) mutant FTN with N-terminal GFP; and
(iv) mutant FTN with N-terminal GFP and C-terminal AuBP.

The sequences of these variants (DNA and protein) are provided herein. These four proteins were purified and their quaternary structure analysed by HPLC (see FIGS. 5 and 6). It is evident from analysis of these data that the 4 mutations introduced into the dimer interface of hFTN successfully destabilise the quaternary structure and the mutated protein elutes as a monomer by SEC. While the 4 mutations introduced into lFTN destabilise the quaternary structure to some degree, there is still a large proportion of 24-mer nanocage still present.

Antibody Binding Domain

As the destabilisation of hFTN worked well, a domain was added to its N-terminus to facilitate its subsequent binding to antibodies. For this purpose the Z-domain was chosen. This is a derivative of Staphylococcus protein A, and is an engineered version of the IgG binding domain of protein A with greater stability and a higher binding affinity for the Fc antibody domain (Nilsson 1987, ref 21). The Z domain was coded as a repeat so that two tandem domains would be present (ZZ). SEC analysis of hFTN with an N-terminal ZZ and GFP demonstrates that the full length protein is still purified as a nanocage, while the mutated hFTN purifies as a monomer (see FIG. 7).

### Example 3

### Reassembly of Human Ferritin Nanocages

Having destabilised the FTN nanocage with the various mutations described in Example 2, the inventors wanted to demonstrate if they could reassemble the nanocage in an ordered manner around a metallic nanoparticle (e.g. gold), as they had done previously with Bfr (see FIG. 3—Example 1). The ZZ-GFP-FTN fusions for both wild type hFTN and mutant hFTN (L29A L36A 181A L83A) were incubated with approximately stoichiometric amounts of gold nanoparticle (AuNP), and examined by size exclusion chromatography (SEC). SEC separates proteins and complexes based on their size, where smaller molecules have a longer path through the porous column matrix and elute slower, whereas larger molecules elute quicker as they spend more time in the void volume. This can be used to very effectively separate the ferritin monomer from the cage complexes (see FIG. 2). Both the wild type (see FIG. 8) and the mutant hFTN (L29A L36A 181A L83A) (see FIG. 9) demonstrated a higher order complex containing both protein and AuNP, which appeared to suggest that the AuNP was able to form ordered complexes with both wt and mutated protein.

Further analysis of the AuNP complexes purified by SEC HPLC was performed by transmission electron microscopy (TEM). These data indicate that the wt ZZ-GFP-hFTN protein forms clusters with the AuNPs, but there is no evidence of the AuNP being encapsulated within the hollow space of the ferritin (see FIG. 10A). The wt ZZ-GFP-hFTN alone readily forms isolated nanocage structures (see FIG. 10D). The ZZ-GFP-hFTN (L29A L36A 181A L83A) mutant does not form nanocages in the absence of AuNP (see FIG. 10C), but in the presence of AuNP there is a high proportion of nanocage structures where the AuNP is clearly encapsulated within the central space of the ferritin nanocage (see FIG. 10B).

These data clearly demonstrate that the L29A L36A 181A L83A mutations introduced at the dimer interface of hFTN are sufficient to destabilise the protein interface so that it does not form the quaternary nanocage structure. The surprising and unpredicted result is that this destabilised protein will template around a AuNP to form nanocage structures that encapsulate the AuNP with a high degree of efficiency. This is particularly surprising because the template occurred without the need to include a gold binding peptide on the interior C-terminus of the FTN, as was previously required for Bfr (see FIGS. 2 and 3).

### Example 4

### Encapsulation of Drugs into the Nanocages

In Example 3, the inventors have demonstrated the ordered assembly of the ferritin nanocages around a gold nanoparticle. They have also used this programmed ordered assembly to enable the direct encapsulation of drugs inside the nanocages. Gold nanoparticles have been considered as stand-alone vectors for drug delivery through the formation of covalent drug-Au conjugates²⁰. Here they sought to exploit a different approach using passive binding of drug molecules to the highly polarisable Au surface and stabilisation through their subsequent encapsulation in the ferritin nanocage. The inventors evaluated the binding of the anti-cancer drug doxorubicin (Dox) to 5 nm Au nanoparticles through its intrinsic fluorescence. Quenching of the fluorescence in the presence of Au nanoparticles demonstrates an interaction between the Dox and the Au (see FIG. 11). In addition, they demonstrated an interaction between propidium iodide (PI) and Au nanoparticles, and in this instance a complete ablation of fluorescence was observed (see FIG. 12).

Since small molecules can bind to Au nanoparticles, they hypothesised that this would provide a mechanism for the ordered encapsulation of the drugs into protein nanocages, since they have demonstrated that the nanoparticles can form an ordered structure around the Au nanoparticles. The inventors therefore sought to demonstrate that prior binding of small molecules to Au nanoparticles will lead to their encapsulation within a protein nanocage with the nanocage formation being directed by the Au-drug nanoparticle conjugate. To evaluate this, the mutant hFTN (L29A L36A I81A L83A) protein was added to the Au nanoparticles in the presence of different concentrations of Dox or PI. The nanocages that were formed around the Au nanoparticle were then purified by HPLC (as in FIG. 9). The purified Dox-Au-nanocage complex was then evaluated for Dox by measurement of Dox fluorescence. The clear presence of Dox fluorescence indicated that Dox was present in the purified nanocage complexes (see FIG. 13). Encapsulation of PI by fluorescence could not be monitored due to its complete quenching on binding.

Further analysis of drug encapsulation was evaluated by mass spectrometry (MS). Complexes of drug-Au-nanocage were purified by HPLC prior to analysis by MS to determine if the drug was present in the complex. Data clearly demonstrate that both PI and Dox were present in the nanocage complex and that encapsulation of the drug occurred with both citrate and PBS stabilised Au nanoparticles (see FIG. 14). Together these data demonstrate that passive binding of small molecules to the Au nanoparticles is sufficient to direct their encapsulation into the ferritin nanocages.

### Example 5

### Targeting of Ferritin Nanocage to Target Cells

Ferritin fusions containing an N-terminal ZZ domain, in principle, should be able to bind to IgG isotype antibodies since the Z-domain is a synthetic derivative of an IgG binding domain from Staphylococcus aureus protein A. The inventors evaluated the specificity with which they can direct the targeting of the ferritin nanocage to specific cell types by direct antibody interactions. To establish a fluorescent basis for determining cell binding they used the GFP labelled wt ZZ-GFP-hFTN. Two different cell types and antibodies were used to demonstrate the principle of cell-specific targeting, here they chose MNK1.1 (mouse natural killer cells) and HT29 (colorectal cancer) cell lines, which have known antibodies that can either target the NK1.1 receptor in the case of MNK1.1 or the EGFR receptor in the case of HT29. Flow cytometry studies with wt ZZ-GFP-hFTN in the presence or absence of the appropriate targeting antibody demonstrate no discernible background binding of the nanocage in the absence of antibody, whilst a complete shift in the fluorescence of the population was observed in the presence of the antibody (see FIG. 15).

### Example 6

### Delivery of Drugs to Target Cells

Having demonstrated that the nanocage can effectively be targeted to specific cells by prior binding to an antibody exhibiting immunospecificity to such cells, the inventors sought to determine that the drug-loaded nanocage complex could deliver a payload of drugs to cells. Nanocages with GFP were created to monitor the delivery and fate of the nanocage in cells, while ferritin without GFP was used to create nanocages with Au-drug encapsulated so that the fate of the drug could be monitored by fluorescence. Au-ZZ-GFP-hFTN (L29A L36A I81A L83A) and Drug-Au-ZZ-hFTN (L29A L36A I81A L83A) complexes were formed as before and purified by HPLC. They were then mixed with anti-EGFR as before and their interaction with HT29 cells was monitored over time.

The GFP-labelled nanocages were clearly seen to bind to the cells and after 2 h punctate distributions of nanocages could be observed both on the surface and inside the cells (FIG. 16). Cells were also stained with lamps, a late lysosomal marker. The internalised GFP signal after 2 h can clearly be seen to be punctate but not associate with lysosomes, consistent with early stage endocytosis into endosomes (see FIGS. 16a and 16b). After 24 h, the picture clearly changed, with GFP being dispersed throughout the cell cytoplasm and partly associated with lysosomal signal, consistent with it being broken down and dispersed by the pH drop associated with lysosomes (see FIGS. 16d and 16e).

The ability of drug-loaded nanocage to deliver drug to cells was monitored by following the fluorescence signal of Dox. Purified Dox-Au-ZZ-hFTN (L29A L36A I81A L83A) was incubated with cells and imaged after 2 h and 24 h for Dox fluorescence with combined DAPI staining of nuclei. After 2 h, there is a weak signal of Dox in the cytoplasm, but Dox bound by the Au-nanoparticle will have significantly reduced fluorescence based on our previous characterisation. After 24 h, there is a clear translocation of Dox signal to the nuclei of cells (see FIG. 17). This is consistent with the fate of the nanocage observed in FIG. 16, with dispersal of the nanocage leading to dispersal of the Dox and its translocation to the nucleus.

Attempts to observe delivery of PI by confocal microscopy did not successfully observe PI (see FIG. 18). The only signal from the PI channel was also observed with the cell only control and is consistent with auto-fluorescence (note that PI is imaged at a different wavelength to Dox).

Further evaluation of drug delivery was performed by mass spectrometry. Following the dosing procedure used above, cells were washed prior to lysis and drug presence measured by LC-MS (Agilent 6550). Both PI and Dox delivered by the nanocage were present in the lysed cells (see FIG. 19). It was also possible to see the delivery of drugs alone in the control samples, where free drug concentrations were used that were the same as the concentrations used when making the nanocage-drug conjugates (50 μM for PI and 2 μM for Dox). Cells that were treated with the nanocage alone did not give any signal by mass spectrometry (not shown).

### Example 7

### Phenotypic Assay of Drug Delivery to Cells

The inventors have used phenotypic assays to demonstrate the effective delivery of Dox into cells. The MTT assay measures the metabolic activity of cells via NAD(P)H dependent oxidoreductase enzymes using a tetrazolium dye substrate (MTT) that produces a purple colour on reduction. A reduced numbers of viable cells leads to a loss of activity and hence a reduced colour response. Au-ZZ-GFP-hFTN (L29A L36A I81A L83A) and Dox-Au-ZZ-hFTN (L29A L36A I81A L83A) complexes were formed as before and purified by HPLC. In the case of the Dox loaded nanocages, two concentrations of Dox (0.1 μM & 0.2 μM) were used when forming the complexes. They were then mixed with anti-EGFR as before and their interaction with HT29 cells was monitored over time prior to measuring viability using the MTT assay. The nanocages that were formed with the higher loading of Dox clearly demonstrated a phenotypic response during the time course of the assay (FIG. 20a). The data also demonstrate a dose response to the different nanocage loading conditions used of Dox (0.1 or 2.0 μM). A further phenotypic assay was performed using flow cytometry and the Topro3 dye. Topro3 binds to DNA and preferentially enters non-viable cells. As before, HT29 cells were treated with Au-ZZ-GFP-hFTN (L29A L36A I81A L83A) and Dox-Au-ZZ-hFTN (L29A L36A I81A L83A) complexes pre-bound to the anti-EGFR antibody; a control of Dox only was also performed along with cells only (FIG. 20b). In this assay the drug loaded nanocage demonstrates a clear difference in viability at 24 h. The difference with the control cells becomes less pronounced at longer time points, and this may be due to uptake being triggered by the presence of the anti-EGFR antibody. It is also known that at longer time points this dye becomes less specific as a viability signal, although the cell only control has a low response even after 72 h.

### Example 8

### Using the Nanocage in a Phenotypic Screening Platform

The inventors have demonstrated the ability to use the ferritin nanocage as a platform technology for the delivery of small molecule drugs into cells. Because the technology provides a defined process for the encapsulation and assembly of the nanocage complex, it can be envisioned as a generic method for the delivery of compounds into cells. The binding of small molecule compounds to the Au nanoparticle will work for a wide variety of ionic, electrostatic and hydrophobic interactions. The assembly of the mutant nanocage around the drug-bound nanoparticle also appears robust. Further, the binding of the nanocage complex to an antibody by interaction of the ZZ domain with IgG isotype antibodies is fast and effective. This can therefore be applied to a very wide range of commercially available antibodies and so can be used to effectively target a wide range of different cell types.

Because of the ordered process and versatility of nanocage delivery, it is possible to use this as a platform for screening small molecules for in vivo efficacy. In many instances small molecule drugs fail because of poor cell permeability. Furthermore, during drug development conclusions are frequently made regarding efficacy of classes of compounds in phenotypic cell assays but without any knowledge of cell permeability; the drugs may be highly effective if they can be made to cross the cell membrane. Being able to further delineate the mode of failure, non-cell penetration, or poor biological effectiveness, would be valuable in screening campaigns.

The ferritin nanocage described herein provides a methodology for the effective delivery of compounds into cells in a phenotypic assay and the ordered assembly process is adaptable to high throughput screening scenarios. Furthermore, nanocages that are made fluorescent, either through chemical labelling, or the fusion of fluorescent proteins, can be used to monitor the uptake of individual cells. When combined with cell sorting methods the phenotypic assays could be correlated to a dose response based on the nanocage fluorescence.

### Example 9

### Nanocages in the Diagnosis and Treatment of Disease

The ability to target ferritin nanocages to specific cell types via the binding of antibodies creates possibilities for the diagnosis and treatment of disease. Because the nanocages can be made fluorescent, they can be used in imaging methods to identify specific cell types displaying known epitope disease markers. This creates possibilities for their use in the diagnosis of cancer types in imaging accessible locations. Examples of this are cancers accessible via GI-tract, such as oesophageal, stomach, colorectal, liver, pancreatic, gall bladder. In addition, cancers near to the surface of the body would be accessible for diagnosis including skin cancer and neck and throat cancers.

The ability to encapsulate drugs into the nanocage also provides the possibility of combined diagnostic and therapy (theranostic) approaches. Furthermore, because the drug encapsulated complex contains an Au nanoparticle, a mechanism for the activated release of drugs is also possible. Au nanoparticles absorb light due to their plasmonic effect and laser irradiation is proven to cause localised heating of the nanoparticle proportional to the intensity of the incident laser irradiation (Honda et al). Following targeting of the nanocage, laser induced heating may therefore be used to activate the release of the encapsulated drug, since localised heating will lead to the thermal disassembly of the nanocage complex. This type of approach can make use of current endoscope technology that can both locally deliver compounds, image and treat using laser light sources. The inventors therefore consider that this type of nanocage device would fit with current therapeutic practices and approaches.

### Example 10

### Measuring Drug Release by Fluorescence Polarisation

The principle of laser-induced drug release can be demonstrated by examining the fluorescence polarisation of a fluorescently bound molecule within the nanocage, such as Dox. Anisotropy provides an intensity independent measure of the degree of polarisation within a sample. Briefly, when a fluorescent molecule absorbs plane polarised light, it will be emitted in the same plane as the excitation source. However, during the fluorescence lifetime, between absorption and emission, the molecule may rotate. This means that the emitted light will be relative to the new orientation of the molecule. By measuring the emitted light in both vertical and horizontal planes, it is possible to determine the degree of polarisation (anisotropy). Because large molecules rotate slower than small molecules, the degree of anisotropy will be dependent on the size of the molecule. A fluorescent molecule encapsulated in the nanocage will therefore have a very high anisotropy value. If laser irradiation of the Au nanoparticle leads to breakdown of the nanocage and release of a fluorescent compound, this will be imaged by a significant reduction in the measured anisotropy.

### Example 11

### Delivery of Paclitaxel to Cells by Ferritin Nanocage

Paclitaxel (Pac) is a natural product, first isolated from the Pacific yew tree. It is commonly used to treat many types of cancer and is known to have many side effects. It prevents cell division by targeting mitotic spindle assembly. An albumin bound formulation (abraxane) has, to a degree, enhanced the efficacy of the drug in cancer treatment, and alleviated some of the toxicity issues associated with the solvent previously used for administration. Abraxane demonstrates, in principle, the advantages that can be obtained for appropriate drug delivery, but it still has significant toxicity issues.

The inventors have performed experiments to demonstrate the encapsulation and delivery of Pac by the ferritin nanocage of the invention to a colon tumour cancer cell line—HT-29. Pac (5 μM) was encapsulated to create Drug-Au-ZZ-hFtn(L29A L36A L81A L83A) nanocages as described above. Excess free drug was removed using a Zorbax spin column prior to addition to cells. Anti-EGFR antibody (0.5 μg) was added to the Pac-Au-ZZ-hFtn(L29A L36A L81A L83A) and the antibody bound cage added to cells (30 nM).

The unloaded Au-ZZ-hFtn(L29A L36A L81A L83A) delivery vehicle was added to HT29 cells as a control to determine cytotoxic effects of hFtn that only contained gold nanoparticles. Free Pac was added to cells at high concentration (5 μM) as a drug only control. The phenotypic effect of the delivery of drugs into cells was assessed via Vybrant fluorescent staining using flow cytometry to measure percentages of dead, apoptotic and live cells.

After 48 h hFtn-Pac (>70% cell death) can be delivered into cells to release a payload of paclitaxel that causes surprisingly more cell death than the free drug alone (˜9% cell death) (see FIG. 21). These data demonstrate that the hFtn nanocage is highly efficient at encapsulating and delivering Pac into cells in the presence of an appropriate targeting antibody. Pac causes significant cellular toxicity leading to a strong phenotypic response when delivered, while free Pac, which has poor membrane permeability, has very little effect on cells.

### Example 12

### Delivery of Actinomycin-D to Cells by Ferritin Nanocage

Actinomycin-D (Act-D) consists of two cyclic peptides linked via a phenoxazone ring. It is an antibiotic that is also used as a chemotherapy medication to treat a number of types of cancer and is on the WHOs list of essential medicines. It has significant side effects.

The inventors performed experiments to discover if a cyclic peptide of the size and complexity of Act-D could be encapsulated and delivered to cells by the ferritin nanocage of the invention to a colon tumour cancer cell line—HT-29. Act-D (5 μM) was encapsulated to create Drug-Au-ZZ-hFtn(L29A L36A L81A L83A) nanocages as before. Excess free drug was removed using a Zorbax spin column prior to addition to cells. Anti-EGFR antibody (0.5 μg) was added to the Act-D-Au-ZZ-hFtn(L29A L36A L81A L83A) and the antibody bound cage added to cells (30 nM).

The unloaded Au-ZZ-hFtn(L29A L36A L81A L83A) delivery vehicle was added to HT29 cells as a control to determine cytotoxic effects of hFtn that only contained gold nanoparticles. Free Act-D was added to cells at high concentration (5 μM) as a drug only control. The phenotypic effect of the delivery of drugs into cells was assessed via Vybrant fluorescent staining using flow cytometry to measure percentages of dead, apoptotic and live cells. After 48 h Act-D-Au-ZZ-hFtn(L29A L36A L81A L83A) (10% cell death) can be delivered into cells to release a payload of Act-D. The free drug alone at high concentration causes a similar degree of cell death to what we have observed with the nanocage delivered drug (see FIG. 22). It is thus evident that the free drug has some cell penetrating properties that lead it to enter into the cell and cause death, although, in the described assay, the degree of cell killing was not as high as that reported in the literature for a similar concentration²². Treatment with 30 nM Act-D loaded nanocage gave a similar response to high concentrations of free drug demonstrating that a similar level of cellular delivery was achieved with substantially lower concentrations. It appears that Act-D is not as potent at inducing cell death as Pac (compare FIGS. 22 and 21).

### Example 13

### Compound Nanocages

Compound nanocages composed of different types of subunit were also created by incubating the Au nanoparticle with His-ZZ-hFtn(L29A L36A L81A L83A) and His-GFP-hFtn(L29A L36A L81A L83A). Since the Au nanoparticle acts as the nucleating agent and the hFtn part of the fusion protein is identical, nanocages formed that contain the ZZ domain on some subunits and the GFP domain on others. These compound nanocages behaved as expected in terms of fluorescence and cellular delivery of drugs.

### Example 14

### Mass Spectrometry Drug Quantification

To demonstrate that the loading of drug in the nanocage could be performed, mass spectrometry of the purified Act-D-His-ZZ-hFTN(L29A L36A I81A L83A) nanocage was performed. Calibration curves are necessary for the direct quantitation of samples. For the protein component, a peptide fragment was identified that provided a good readout of hFtn(L29A L36A I81A L83A) monomer concentration; standard dilutions were then used to create a standard curve based on the 50 ppm area of the m/z signal for this peptide. Similarly a standard curve for the Act-D was established based on standard dilutions of Act-D bound to Au nanoparticles in case this affected the ability of the Au-nanoparticle to resolve the Act-D signal.

Both standard curves provided good linear responses to concentration (see FIG. 23 A&B). Based on these calibration curves a purified ZZ-Au-hFTN (L29A L36A I81A L83A) was analysed. The quantitation of signal arising from ZZ-Au-hFTN (L29A L36A I81A L83A) and Act-D was then calculated based on the standard curves. Following correction for monomer to nanocage formation, the amount of Act-D was calculated as 13.3 molecules per nanocage (see FIG. 23C).

### REFERENCES

1. He, D. and J. Marles-Wright (2015). New biotechnology 32(6), 651.

2. Webb, B., J. Frame, et al. (1994). Archives of biochemistry and biophysics 309(1), 178.

3. Simsek, E. and M. A. Kilic (2005). Journal of Magnetism and Magnetic Materials 293, 509.

4. Liang, M., K. Fan, et al. (2014). Proceedings of the National Academy of Sciences of the United States of America 111(41), 14900.

5. Uchida, M., M. L. Flenniken, et al. (2006). Journal of the American Chemical Society 128(51), 16626.

6. Millard, M., S. Odde, et al. (2011). Theranostics 1, 154.

7. Niu, G. and X. Chen (2011). Theranostics 1, 30.

8. Ye, Y. and X. Chen (2011). Theranostics 1, 102.

9. Liu, Z., B. Jia, et al. (2011). Molecular imaging and biology: MIB: the official publication of the Academy of Molecular Imaging 13(1), 112.

10. Zhen, Z., W. Tang, et al. (2013). ACS nano 7(6), 4830.

11. Lin, X., J. Xie, et al. (2011). Nano Lett 11(2), 814.

12. Wilhelm, S., A. J. Tavares, et al. (2016). Nature Reviews.

13. Dvorak, A. M., S. Kohn, et al. (1996). Journal of leukocyte biology 59(1), 100.

14. Feng, D., J. A. Nagy, et al. (1996). The Journal of experimental medicine 183(5), 1981.

15. Cheung-Lau, J. C., D. Liu, et al. (2014). Journal of inorganic biochemistry 130, 59.

16. Swift, J., C. A. Butts, et al. (2009). Langmuir: the ACS journal of surfaces and colloids 25(9), 5219.

17. Dominguez-Vera, J. M. and E. Colacio (2003). Inorganic chemistry 42(22), 6983.

18. Kim, M., Y. Rho, et al. (2011). Biomacromolecules 12(5), 1629.

19. Brown, S. (1997). Nat Biotechnol 15(3), 269.

20. Vigderman, L. and E. R. Zubarev (2013). Advanced drug delivery reviews 65(5), 663.

21. Nilsson, B., T. Moks, et al. (1987). Protein engineering 1(2), 107.

22. Lu, D. F., Y. S. Wang, et al. (2015). International journal of clinical and experimental medicine 8(2), 1904.

1. A variant ferritin polypeptide comprising a modified amino acid sequence of a wild-type ferritin polypeptide, the modified sequence being in a dimeric subunit interface or the N-terminus of the polypeptide, wherein the variant is incapable of assembling into a ferritin nanocage unless it is contacted with a nucleating agent.

2-10. (canceled)

11. A polypeptide according to claim 1, wherein the variant ferritin polypeptide comprises a variant human heavy chain ferritin.

12. A polypeptide according to claim 11, wherein the variant human heavy chain ferritin comprises one or more modification in the wild-type polypeptide, wherein one or more hydrophobic residue in the heavy chain dimeric subunit interface of the polypeptide is substituted with a small amino acid residue, thereby rendering the variant incapable of forming heavy chain dimers, and hence higher order nanocages, unless it is contacted with a nucleating agent and wherein the heavy chain dimeric subunit interface comprises or consists of amino acid residues as set out in SEQ ID No: 19, 20, 21, 22 or 29.

13. (canceled)

14. A polypeptide according to claim 11, wherein the variant heavy chain ferritin polypeptide comprises at least one, two, three or four modification in amino acids 29, 36, 81 or 83 of SEQ ID No:16.

15. A polypeptide according to claim 11, wherein the variant heavy chain ferritin polypeptide is formed by modification of amino acid residue L29, L36, I81 and/or L83 of SEQ ID No:16, wherein the modification at amino acid L29 comprises a substitution with an alanine, the modification at amino acid L36 comprises a substitution with an alanine, the modification at amino acid I81 comprises a substitution with an alanine, and/or the modification at amino acid L83 comprises a substitution with an alanine.

16. A polypeptide according to claim 11, wherein the variant human heavy chain ferritin polypeptide is encoded by a nucleic acid (SEQ ID No:30) or comprises an amino acid (SEQ ID No:31) sequence, or fragment of variant thereof.

17-32. (canceled)

33. A polypeptide according to claim 1, wherein the variant ferritin comprises an amino acid sequence configured to bind to an antibody or antigen binding fragment thereof, optionally wherein the antibody or antigen binding fragment thereof binding peptide is disposed at or towards the N-terminus of the variant ferritin polypeptide.

34. A polypeptide according to claim 33, wherein the antibody or antigen binding fragment thereof binding amino acid sequence comprises a Z-domain, optionally wherein the Z domain sequence is coded as a repeat so that two tandem domains are disposed adjacent to one another (i.e. ZZ).

35. A polypeptide according to claim 34, wherein the Z-domain is encoded by the nucleic acid sequence (SEQ ID No:48) or comprises the amino acid sequence (SEQ ID No:49), or fragment or variant thereof.

36. A polypeptide according to claim 33, wherein the variant human heavy chain ferritin is encoded by a nucleic acid (SEQ ID No:50) or comprises an amino acid (SEQ ID No:51) sequence, or fragment or variant thereof.

37. (canceled)

38. A fusion protein comprising wild-type ferritin and one or more peptide selected from a group consisting of: an antibody or antigen binding fragment thereof binding peptide; a fluorophore; a His tag; and a nucleating agent binding peptide, wherein the antibody or antigen binding fragment thereof binding peptide is as defined in claim 35.

39. (canceled)

40. (canceled)

41. (canceled)

42. (canceled)

43. A ferritin nanocage comprising the variant ferritin polypeptide according to claim 1 and a nucleating agent.

44. (canceled)

45. A nanocage according to claim 43, wherein the nucleating agent comprises a nanoparticle having an average diameter of about 1-500 nm, 1-100 nm, 2-50 nm, or 3-10 nm.

46. A nanocage according to claim 43, wherein the nucleating agent is metallic, optionally wherein the nucleating agent is gold, iron, or copper.

47. A nanocage according to claim 43, wherein the ferritin nanocage encapsulates a gold nanoparticle.

48. (canceled)

49. A nanocage according to claim 43, wherein the ferritin nanocage comprises or is functionalised with an antibody or antigen binding fragment thereof, optionally wherein the antibody or antigen binding fragment thereof is immunospecific for endocytic receptors or an IgG antibody.

50. A nanocage according to claim 43, wherein the nucleating agent is bound to a payload molecule which is an active agent, such as a drug molecule.

51. (canceled)

52. (canceled)

53. (canceled)

54. A method of encapsulating a payload molecule, preferably a drug molecule, in a ferritin nanocage, the method comprising contacting the variant ferritin polypeptide according to claim 1 with a nucleating agent conjugated to a payload molecule and allowing the polypeptide or protein to self-assemble into a nanocage, thereby encapsulating the payload molecule.

55. A nanocage according to claim 50, wherein the molecular weight of the payload molecule is 50 Da to 10 kDa.

56-65. (canceled)
